(19) United States
(12) Patent Application Publication
(10) Pub. No.: US 2024/0352410 A1
(43) Pub. Date: Oct. 24, 2024

(54) NANOFIBERS AND THEIR USE IN ENHANCING PARTICLE-BASED HYDROGEL SCAFFOLDS FOR REGENERATIVE MEDICINE AND TISSUE ENGINEERING

(71) Applicant: UNIVERSITY OF VIRGINIA PATENT FOUNDATION, Charlottesville, VA (US)

(72) Inventors: Christopher Bruce Highley, Crozet, VA (US); Jack Whitewolf, Charlottesville, VA (US); M. Gregory Grewal, Charlottesville, VA (US)

(21) Appl. No.: 18/642,539

(22) Filed: Apr. 22, 2024

Related U.S. Application Data
(60) Provisional application No. 63/460,832, filed on Apr. 20, 2023.
(60) Provisional application No. 63/526,646, filed on Jul. 13, 2023.

(51) Int. Cl. C12N 5/00; B33Y 10/00

(52) U.S. Cl. CPC C12N 5/0068; B33Y 10/00; C12N 2535/00; C12N 2537/00; C12N 2539/00

(57) ABSTRACT

Provided are stable hydrogels systems with polymeric fibers and method of preparation thereof. Advantageously, the present hydrogel systems can have improved stability without the need of covalent crosslinking between the fibers. Due to their unique mechanical properties and stability, the present hydrogel systems may be useful for a wide variety of applications, including 3D printing, tissue engineering, regenerative medicine, drug delivery, and implantation.

### CROSS-REFERENCE TO RELATED APPLICATIONS

The present application claims priority to U.S. Provisional Patent Application Nos. 63/460,832 filed on Apr. 20, 2023, and 63/526,646 filed on Jul. 13, 2023 of which are hereby incorporated by reference in their entireties.

### STATEMENT REGARDING FEDERALLY SPONSORED RESEARCH

This invention was made with government support under 2036968 awarded by the National Science Foundation and GM147410 awarded by the National Institutes of Health. The government has certain rights in the invention.

### FIELD OF THE INVENTION

The disclosed technology is generally directed to nanofibers and their use in enhancing particle-based hydrogel scaffolds for regenerative medicine and tissue engineering.

### BACKGROUND OF THE INVENTION

Particle-based hydrogels are a rapidly emerging class of materials that have applications in regenerative medicine, tissue engineering, and biofabrication (i.e., 3D (bio)printing). A macroscale (millimeter scale and larger) volume of these materials consists of individual microscale particles (generally 200 m or less) in characteristic lengths. Typically, these particles are spherical, and the characteristic length is the diameter. Macroscale materials whose volumes consist of these microscale particles are sometimes referred to as granular materials, where the individual, discrete particles might be considered grains. In the case of particle-based hydrogels, the individual particles are generally microscale hydrogels (microgels) formed via emulsification processes (including batch emulsification or microfluidic particle generation). By concentrating these particles (removing much or all of the liquid between them), they come into physical contact. Macroscale, collective bulks can be stabilized by interparticle crosslinks or, when enough fluid between the particles is removed, by simple physical interactions between particles in jammed systems. In the latter case, bulk granular hydrogels are static when the stress applied to them is below a characteristic yield stress, but they will flow with ease when stress applied to the bulk (or to a specific location in the bulk) exceeds the yield stress. These properties are extremely desirable in bioprinting (where materials must flow during extrusion and rapidly stabilize after) and in regenerative medicine applications where injectable materials are desirable. Additionally, porosity can be controlled through the amount of fluid included between particles, which has been seen to be advantageous in regenerating tissue. High porosity is important in applications of granular hydrogels where cells are incorporated withing the granular material or when the granular material is intended to support or promote cellular migration or ingrowth into its volume.

The bulk mechanical integrity of a granular hydrogel is derived in large part from particle-particle interactions, often through chemical bonds between microparticles. When these bonds are weak, or when there is limited particle-particle contact, as in the case of increasingly porous granular materials, the structure of the granular material can be compromised, which can result in its falling apart or condensing and losing porosity. Thus, there is remain a need for hydrogels that maintain stability and desirable mechanical properties at relatively high porosity.

### SUMMARY OF THE INVENTION

In one aspect, the present disclosure provides a scaffold comprising polymeric fibers having an aspect ratio of at least 20 (such as 30 or higher) and hydrogel microparticles, wherein the polymeric fibers and the hydrogel microparticles form a stable network. In some embodiments, the scaffold has a porosity of at least 30%. In some embodiments, the polymeric fibers of the scaffold are not crosslinked. The polymeric fibers can be, for example, electrospun fibers. The scaffold can comprise about 10% to about 80% by volume polymeric fibers.

In another aspect, the present disclosure provides a method of producing a stable scaffold, comprising mixing polymeric fibers having an aspect ratio of at least 20 and hydrogel microparticles, thereby the polymeric fibers and the hydrogel microparticles form a stable network.

In another aspect, the present disclosure provides a method of culturing cells, which comprises mixing the cells with the scaffold as described herein, thereby the cells are embedded in the scaffold, and culturing the cells embedded in the scaffold.

In another aspect, the present disclosure provides a hydrogel comprising polymeric fibers having a mean length of at least 35 μm, wherein the hydrogel is stable and has a porosity of at least 30%. (such as 50%, 80%, or higher). In some embodiments, the polymeric fibers of the hydrogel are not crosslinked. In some embodiments, the polymeric fibers of the hydrogel have an aspect ratio of at least 15. The hydrogel can comprise about 10% to about 80% by volume the polymeric fibers.

In another aspect, the present disclosure provides a scaffold comprising the hydrogel as described herein.

In another aspect, the present disclosure provides an ink or filament for 3D printing comprising the hydrogel as described herein. A scaffold for cell culture can be produced by printing the ink or filament as described herein using a 3D printer.

In another aspect, the present disclosure provides a method of preparing a hydrogel, which can comprise electrospinning a polymer into a polymeric fiber having a mean length of at least 35 μm; and hydrating the polymeric fiber in an aqueous medium to form a stable hydrogel having a porosity of at least 30%.

In another aspect, the present disclosure provides a method of culturing cells, which can comprise mixing the cells with the scaffold having the hydrogel as described herein or the 3D printed scaffold as described herein, thereby the cells are embedded in the scaffold; and culturing the cells embedded in the scaffold.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

The patent or application file contains at least one drawing executed in color. Copies of this patent or patent application publication with color drawing(s) will be provided by the Office upon request and payment of the necessary fee.

FIGS. 1A-1B show A) Thiol-ene click chemistry using norHA and DTT. Norbornenes conjugated to HA and DTT can be crosslinked under UV light with the presence of a photoinitiator (LAP). Stoichiometric control of DTT ensures there be excess norbornene for later reactions. B) HMP fabrication. HMP precursor (norHA, DTT, and LAP; aqueous phase) is mixed together with mineral oil with 2% Span80 (oil phase) and homogenized to form an emulsion. Application of UV light while stirring crosslinks the precursor solution into stable HMPs.

FIGS. 2A-2C show NorHA HMP size characterization. A) Size distributions comparing HMPs made from varying homogenization spin speeds. B) Size distributions comparing HMPs with varying compositions. C) Fluorescent micrograph of HMPs at varying spin speeds. Scale bars=100 μm.

FIGS. 3A-3D show properties of granular hydrogels—here, specifically those formed from the particles used in fiber-reinforced scaffolds—are determined by the formulation of the hydrogel used in the particles as well as the particle sizes that result from the emulsification process. Here, the storage modulus of a granular hydrogel is seen to be a function of polymer concentration, but not particle size. Yield stress of the granular material is seen to be a function of particle size and polymer concentration. These properties are important in the application of granular materials, and would extrapolate, in various ways, to fiber reinforced materials. Most specifically, these observations would best extrapolate to granular materials during preparation and inject, prior to the removal of any removable particle component (such as gelatin particles). A) In granular hydrogels formed from particles, storage modulus increases with polymer concentration, but B) is not dependent on the particle size used in forming the granular material. However, C) yield stress is observed to be depended on particle size D) as well as on particle polymer concentration. C) Yields stresses of granular materials tend to increase as particle sizes decrease. D) Similarly, yield stresses increase as polymer concentration increases. However, an notable exception are granular hydrogels formed from 2 wt % polymer particles. In this case, yield stress is independent of size.

FIGS. 4A-4B show A) HMP scaffold fabrication. Desired ratios of norHA and gelatin HMPs, along with DTT and LAP, are combined and packed by centrifugation. The packed HMPs were then crosslinked and cultured at 37° C. to liquefy gelatin HMPs. The scaffolds were then submerged in FITC-dextran solution and imaged. B) Left: Representative confocal images (top) and corresponding thresholded images (bottom) of scaffolds with varying ratios of norHA HMP (750 rpm) to gelatin HMP (2000 rpm). Right: Quantified void volume fraction for each scaffold composition. Scale bars=100 μm.

FIGS. 5A-5C shows pore size comparisons, for each row from left to right: 3000 rpm HMP scaffolds, 1500 rpm HMP scaffolds, and 750 rpm HMP scaffolds. A) Area fraction distribution of the binned pore sizes for all pores. B) Number distribution comparison of binned pore sizes for pores smaller than the largest pore found in 100% scaffolds. C) Area fraction distribution of the binned pore sizes for pores smaller than the largest pore found in 100% scaffolds.

FIG. 6 shows pore sizes increase, pores connect to exterior, and number of pores decreases as pores merge as porosity within the materials increases. This figure demonstrates a range of pores possible as well as size distributions in fiber reinforced gels. The top two rows show increasing pore sizes with more porosity designed, and a decreasing number of isolated, small pores within the material. The third row quantifies, for pore volumes observed, the void volume fractions on the left as a function of designed porosity. In the middle, it can be seen that the distribution of small pores is similar, once the largest pores are removed from consideration, illustrating that the small, interior pores are similar across groups. On the right, for interior pores only, again, similar distributions of void volume fraction are seen. In the bottom row, the percentage of pore volume in constructs of decreasing porosity (from left to right) shows that most pore spaces are connected in the highest porosity materials and, in turn, connect to exterior spaces.

FIGS. 7A-7C shows A) Representative fluorescent image of electrospun norHA fibers. A more dilute population of fibers was used for quantification of fiber sizes. B) Electrospun fiber incorporation into the scaffold. Fibers and desired ratios of norHA and gelatin HMPs were combined, along with DTT and LAP. The mixture is jammed through centrifugation and UV-crosslinked; then submerged in rhodamine B-dextran for imaging. C) Left and middle: 3D confocal image of fibers within the scaffold, and fibers combined with interstitial space (in red) within the scaffold. Right: Representative 2D confocal image of fibers (green) and norHA HMPs (red) in a 50% scaffold. All scale bars=100 μm.

FIGS. 8A-8C shows A) Remaining weight fraction of scaffolds tracked over 28 days normalized to scaffold weight at day 0. B) Visual presentation of effect of fiber amounts on 50% scaffold degradation over a period of 5 days. C) Porosity for 50% scaffold with 10% fiber at day 28 compared to day 0.

FIG. 9 shows Left: Rheological quantification of storage moduli of all scaffolds prior to and after gelatin HMPs liquefy. Right: Quantification of the moduli differences for each scaffold before and after gelatin liquefication

FIGS. 10A-10B shows cells can be included within particle based gels described in this application. Although the schematic in part A only illustrates a single population of spherical particles, this could easily also include fibers or be entirely composed of fibers, and the there could be multiple particle populations. A) To create cell-containing particle-based materials, particles can be mixed with one or more cell types—here in a microcentrifuge tube. This mix of cells and particle-based hydrogel material can be transferred, much like a fluid, injected/extruded, and or placed within a mold or dish and crosslinked. B) cells within these materials stay viable throughout processing and maintain high viability for days after extrusion (or “printing”) as quantified in the bar graph.

FIGS. 11A-F show preparation of granular hydrogel units. A) Electrospun PEGNB fibers were crosslinked, hydrated, and homogenized to segment fibers in a fast, scalable fashion. B) Fluorescent micrograph of segmented PEGNB fibers. C) Quantification of fiber and sphere length illustrating matching of fiber and sphere dimension in spheres (D) group. D) Schematic of a binodal curve for PEG and dextran, where two phases occur in the regime above the curve. The ATPS system was then mixed to form dispersed PEGNB spheres within the continuous dextran phase, the hydrogel microparticles were crosslinked, diluted to form a single phase, and finally washed. E) Fluorescent micrograph of discrete PEG spheres formed using the process illustrated in D. F) Quantification of fiber and sphere diameter illustrates the disparity in the dimensions between fibers and spheres. The aspect ratio of spheres was assumed to be ˜1, so length and diameter was assumed to be equal for these groups. Scalebars in b and e=200 μm; n>300 for all groups.

FIGS. 12A-12E show particle size and shape influences overall granular hydrogel properties. A) Granular hydrogels following centrifuge-mediated packing exhibit void spaces in both a packing density- and particle shape-dependent manner. B) Storage moduli indicate that all groups exhibit solid-like behaviors at low strains, except for Med-Sphere (V) where there was no appreciable storage modulus, likely due to insufficient contact forces between particles. Generally, increased packing density yielded greater elastic contribution in granular hydrogels, with Med-Fiber and Med-Sphere (D) exhibiting similar stiffnesses due to similar characteristic dimensions defining the system. C) Both fiber particle- and spherical particle-based granular hydrogels undergo conversion from solid-like to liquid-like behaviors under application and removal of high strain (exceeding yield strain). Fibers show concentration- (porosity-) dependent behaviors. D) The response of the storage and loss moduli of particle-based materials show a characteristic constant storage and loss modulus, with solid-like behavior until a yield stress is reached, after which point the materials begin to flow like fluids, with loss-modulus dominated responses, but decreasing storage and loss moduli with increasing strains. As previously, the response in the fiber-based materials is concentration dependent. E) Yield strains (the strain at which the granular materials convert from solid-like to liquid-like behaviors, as shown in D are given for the granular materials. Yield strains for all fiber-based materials are greater than for the spherical particle-based hydrogels, and show some concentration dependence.

FIGS. 13A-13C show viscoelasticity and time-dependent stress relaxation of granular hydrogels. A) Schematic of the parallel plate oscillatory shear rheology testing platform. The increased length scale of interactions between fibers enables sliding and reorganization in response to applied strains whereas particles will begin to shift in response to applied strains. B) Plotting loss vs. storage modulus illustrates both viscous and elastic contributions to the mechanics of granular hydrogels. Fiber-based granular hydrogels exhibit storage moduli that are ˜10× loss moduli (illustrated by gray dashed trendline), which is consistent with many natural tissue types. Conversely, Med-Spheres (D) have a lesser viscous contribution and thus deviate from this 10× trend. Med-Spheres (V) illustrate negligible storage and loss moduli. C) Time-dependent stress relaxation profiles at 15% applied strain of granular hydrogels. Fiber-based granular hydrogels are able to dissipate stress over time as fibers slide and reorganize in response to the applied strain as illustrated by the slow decrease in normalized stress (T_{1/2} on the order of 10-100+s). Spheres (D) are unable to reorganize effectively and seemingly shift and fracture before reorganizing into a granular hydrogel as illustrated by the sharp drop in normalized stress (T_{1/2}<1 s).

FIGS. 14A-14B show strain-dependence of granular hydrogel stress relaxation. A) Granular hydrogels demonstrate that (i) Med-Spheres (D) exhibit a stronger strain-dependence on stress relaxation when compared to all packing densities of fiber-based granular hydrogels (ii-iv). Med-Spheres (D) are able to reorganize and dissipate stress when the applied strain is below their yield strain (˜8%), whereas fiber-based granular hydrogels exhibit a more muted relationship between stress relaxation and applied strain, seemingly regardless of their yield strains. B) These relationships are quantified where (i) all groups exhibit increasing max stress with increasing strain, with Med-Spheres (D) generally exhibiting higher max stresses than fibers at low strains. (ii) Interestingly, Med-Spheres (D) do not relax to the same level as fiber-based groups at strains below their yield strain, indicating that they store more stress than fibers during the time scale investigated if the applied strain is not sufficient to cause them to reorganize and flow. However, there is a sharp increase once the applied strain surpasses their yield strain as they begin to flow. Notably, all fiber groups exhibit a modest positive correlation in total relaxation with respect to applied strain. (iii) Finally, consistent with the previous results of total relaxation, T_{1/4} is considerably longer for Med-Spheres (D) when the applied strain is below the yield strain, with a sharp decrease as the applied strain is increased beyond this threshold. Conversely, fiber-based granular hydrogels exhibit a marginal decrease in relaxation time as applied strain is increased.

FIGS. 15A-15F show modular addition of PEGVS fibers to PEGNB fiber-based hydrogels to increase mechanical properties. A) Schematic of incorporating PEGVS fibers into PEGNB fiber-based granular hydrogels. PEGVS fibers form kinetic chains with each other when exposed to UV light, forming a reinforcing structure, while leaving PEGNB fibers essentially unincorporated. This strategy allows PEGNB fibers to theoretically continue to reorganize and respond to external perturbations with PEGVS providing mechanical stability. B) Incorporating PEGVS fibers at low volumes (2.5%-10% v/v) increases granular hydrogel storage moduli for both 10 s and 120 s annealing times compared to 0% PEGVS, with a larger effect at the longer annealing duration. C-D) Annealing PEGVS fibers in these granular hydrogels diminishes the stress relaxation capabilities of the scaffolds in both PEGVS content-dependent and annealing time-mediated fashions. E-F) Annealing all quantities of PEGVS fibers for both annealing times revealed that the max stress of the scaffolds when 15% strain was applied increased with a related reduction in their ability to dissipate stress in response to that applied strain. It is hypothesized that the PEGVS network within the fiber-based granular hydrogels might allow for local stress relaxation at the microscale due to PEGNB fibers sliding and reorganizing, with PEGVS fibers contributing to the elasticity of the scaffold at the macroscale.

FIG. 16 shows MALDI-TOF spectrum of GCDDD-FAM peptide. Confirmation of fluorescent peptide synthesis. Expected molecular weight: 881.8 Da; MALDI-TOF molecular weight: 880.3 Da and 1069.2 Da. 880.3 peak suggests successful synthesis, with some undesired side products (most notably, 1069.2 peak). The 880.3 peak was the highest in magnitude showing that synthesis was sufficient for use in this study.

FIG. 17 shows PEGVS fiber quantification. Comparisons between PEGNB fibers and PEGVS fibers show marginal differences in length and diameter. However, these values were sufficiently close to be considered interchangeable for this study—especially at the low concentrations (% v/v) of PEGVS fibers in the PEGNB fiber-based granular hydrogels (0-10% v/v).

FIG. 18 shows effect of continuous phase concentration (dextran) on resultant microgel diameter. To determine the effect of the continuous phase concentration, the following variables were held constant: 6% w/v PEGNB, 4.2% w/v PEGSH (corresponding to [−SH]:[−NB]=0.7), and 800 RPM stir rate. Originally decreasing microgel diameter as dextran(70) concentration was increased from 25% to 30% w/v was observed, then a marginal increasing trend in diameter with increasing dextran(70) concentration above 30% w/v was observed. This is likely due to the disperse phase aggregating in lower viscosity dextran(70) solutions to form larger droplets, with increasing viscosity (i.e., higher dextran(70) concentrations) supporting larger independent disperse phase droplets. Importantly, 25% w/v was the chosen continuous phase concentration for spheres (D) used in this study and 40% w/v was utilized for spheres (V).

FIG. 19 shows effect of spin rate on resultant microgel diameter. To determine the effect of the spin rate, the following variables were held constant: 6% w/v PEGNB, 4.2% w/v PEGSH (corresponding to [−SH]:[−NB]=0.7), and 30% dextran(70). Overall, microgel diameter decreased with increasing spin rate. 800 RPM was utilized for spheres (D) in this study and spheres (V) were fabricated via vortexing at maximum speed rather than using a stir plate.

FIG. 20 shows effect of disperse phase concentration (PEGNB and PEGSH) on resultant microgel diameter. To determine the effect of the disperse phase concentration, the following variables were held constant: 30% dextran(70) and 800 RPM stir rate. The concentrations shown in the figure correspond to total % w/v between PEGNB and PEGSH, with the [−SH]:[−NB] ratio indicated below. Generally, increasing PEG concentration resulted in marginally larger microgel diameters, except for 9% PEG with [−SH]:[−NB]=0.5, which had the largest diameter due to the lowest crosslinking density allowing for the greatest swelling. 10.2% PEG—6% PEGNB and 4.2% PEGSH—was utilized in this study for all microparticles.

FIGS. 21A-21E show fabrication of packed hydrogel microfiber-based hydrogel scaffolds. A) Dry MeHA fibers containing a fluorophore for visualization immediately following the electrospinning and crosslinking processes. B) Trituration yields fiber segments in solution that are C) on the order of ˜100 μm in length, with diameters on the order of ˜1 μm. D) Packing via centrifugation at 10,000 RCF for 10 minutes yields a packed hydrogel microfiber scaffold that behaves as a bulk solid at rest. E) Schematic illustrating how PHM-100, PHM-90, and PHM-80 scaffolds were assembled, where more PBS added increases the inter-fiber fluid content while conserving total hydrogel microfiber content in each sample. Scalebars (A−B)=100 μm and (C)=1 cm.

FIGS. 22A-22D show rheological characterization and macroscale extrusion of microfiber particle-based hydrogels. A) Cyclical application of high (250%, shaded regions) and low (1%) strains demonstrates shear-thinning and self-healing properties of PHM-100. B) Quantification of recovery after strain shows that PHM scaffolds recover ˜60-70% of their initial modulus prior to the addition of high strain—suggesting that organization of discrete fibers influences overall mechanical properties. C) Amplitude sweep for PHM-100 demonstrates shear-yielding properties of this group with D) yield strains for diluted groups significantly less than PHM-100. * p<0.05; ** p<0.01.

FIGS. 23A-23C show modified FiSER characterizes extensibility of packed hydrogel microfiber scaffolds. A) Percentage stretch to failure of PHM-100, PHM-90, and PHM-80 filaments indicates that all groups can stretch vertically to greater than 2000% of their original height with no statistical significance between groups. B) Representative trends of normal force for each dilution (normalized to PHM-100) illustrates that although percentage stretch to failure is similar for all groups, the original normal force (indicated by filled circles at 0%) decreases as dilution increases. However, normal forces exhibit similar trends once the stretching begins. Dashed lines correspond to the average % stretch to failure values from A. C) Representative images of PHM-100 being stretched using the modified FiSER setup. Scalebar (C)=1 cm.

FIGS. 24A-24B show characterization of viscoelasticity and stress relaxation properties. A) Plot of shear loss modulus versus shear storage modulus of PHMs. Grey dashed line and black dashed line represent where G′ is equivalent to 5× G″ and 10× G″, respectively. Non-crosslinked groups reside near the 5× trend, whereas the addition of secondary annealing to stabilize the material shifts the viscoelasticity to the 10× trend, which is characteristic of many biological tissues. B) Plot of stress relaxation tests where a 15% constant shear strain was applied to the systems and resultant stress was observed as a function of time. All groups exhibit relatively rapid stress relaxation and mimic profiles characteristic of viscoelastic solids. PHM-100, PHM-90, and PHM-80 are compared with the PHM-100 High group.

FIGS. 25A-25D show extrusion of packed hydrogel microfiber inks. A) (i) PHM-100 was extruded at a rate that yields a 2 cm vertical filament that is ˜0.5 mm in diameter. (ii) Following extrusion, the filament was then manipulated to demonstrate the fidelity of PHM-100. The filament was translated 1 cm horizontally before (iii) returning to the original horizontal position. The dashed circle highlights filament sagging due to stretching. (iv) The filament was finally stretched another 2 cm vertically without breaking. B) Macroscale extrusion of PHM-100 across 4 posts of a 2.5×2.5 cm table without secondary crosslinking. These demonstrations highlight the shear-thinning and self-healing properties that are desired for extrusion printing, with the high extensibility and long-range entanglements enabling stretching and filament fidelity at long ranges without additional annealing mechanisms. C) Fluorescent image of the microscale topography of a printed filament illustrating shear-induced alignment (white arrow indicating direction of printing) of the fibers following extrusion. D) Quantification of fiber direction indicates that a high percentage of fibers are aligned in the direction of shear (0 degrees corresponding to the direction of the white arrow). Scalebar (A−B)=1 cm, (C)=500 μm.

FIGS. 26A-26C show extruded PHM scaffolds influence cell culture. A) Process schematic for extruding PHM-100, crosslinking the individual fibers together to stabilize the filament, then seeding C2C12 cells on top of the scaffold. B) Fluorescent micrograph of C2C12 s tagged with AlexaFluor-488 phalloidin for visualization of cell alignment. C) Quantification of cell alignment with 0 degrees corresponding to the direction of shear (white arrow). The microscale topography of the extruded filament induces alignment of C2C12 cells cultured on top of the scaffold.

FIGS. 27A-27D show PHM scaffolds support 3D cell culture. A) Process schematic for culturing C2C12 cells within a PHM-100 support using PDMS wells to contain the scaffolds. B) C2C12 s stained with AlexaFluor-488 phalloidin (1 d) spreading in the non-crosslinked PHM-100 scaffold. C-D) Quantification of projected cell area and cell shape index reveal ranges of cell spreading and circularity, further supporting the permissivity of PHMs at short timescales. Scalebar (B)=500 μm, (E)=100 μm.

FIG. 28 shows quantification of fiber length and diameter. A) Segmented fiber length was determined to be 93±51 μm. B) Segmented fiber diameter was determined to be 1.6±0.3 μm. Fluorescent images of fiber solutions were utilized to quantify fiber segments.

FIG. 29 shows UV crosslinking of PHM-100 High and PHM-100 Low. Representative time sweeps including a UV cure step (shaded region). PHM-100 High, which has the most methacrylate groups available for crosslinking, exhibits a noticeable increase in storage modulus compared to the original pre-crosslinked state and the crosslinked PHM-100 Low as well. Importantly, both groups demonstrate a larger difference between their respective storage and loss moduli following the secondary crosslinking, which yields G′ values that are ˜10× the G″ values.

FIG. 30 shows modeling stress relaxation with a viscoelastic standard linear solid (SLS) model. Stress relaxation profiles of PHM-100, PHM-90, PHM-80, and PHM-100 High (solid lines) with corresponding SLS models (dashed lines). Representative relaxation time constants (τ) are shown adjacent to each group.

FIGS. 31A-31B show fiber alignment during extrusion. The individual fiber segments within the PHM ink experience shear-induced alignment during extrusion printing processes. This microscale topography was demonstrated to provide contact guidance to C2C12 cells in the main text (FIG. 26). This fiber alignment continues when the direction of extrusion changes. Shown in A) and expanded in B), fiber alignment continues with the curve as the direction of printing follows the white arrow in (A). It is important to note that the depicted filaments contain some aggregates of fibers. Overall, this demonstrated control over microscale topography enables the ability to arbitrarily define the surface topography of a substrate purely by extrusion design. Scalebars=500 μm.

FIG. 32 shows representative rheology results for all norHA HMP compositions and sizes. Left column: time sweeps with strains alternating from high (500%) strain to low (1%) strain. Right column: strain sweeps from 0.1% strain to 500% strain. From top to bottom: rheology results of small, medium, and large norHA HMPs, respectively.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a,” “an,” and “the: include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising,” “including,” or “having” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “aspect ratio” as used herein refers to a ratio between the length and diameter (or width) of a polymer molecule or a physical assembly of polymers or molecular components by processing into a nano- or microscale structure. The length and diameter (or width) of a polymer molecule or nano- or microscale structure can be measured by known analytical means and are generally understood in the art as characteristics of the shape of the molecule or nano- or microscale structure that is formed by processes including electrosprinning or molecular self-assembly. The value of aspect ratio is at least 1, as the greatest measurement among all dimensions is defined as the length of the molecule or nano- or microscale structure. For example, a polymer or nano- or microscale structure with a length of 100 μm and a diameter of 2 μm has an aspect ratio of 50. Polymeric or nano- or microscale structure with a relatively high aspect ratio (e.g., 20 or greater) can be referred to as “fiber” or “fibrous.” Polymeric or fibrous structures with a relatively low aspect ratio (e.g., 5 or less) can be referred to as “microparticle” or “spherical.”

The term “hydrogel microparticles” (or HMPs) refers to hydrogel polymers or nano- or microstructures with an approximately spherical shape. The hydrogel microparticles as used herein typically have an aspect ratio of less than 5, such as about 1, about 1.5, about 2, about 2.5, about 3, about 3.5, about 4, or about 4.5.

The term “porosity” refers to volume percentage of void spaces in a material. For the polymer materials (such as a scaffold or a gel) used herein, the void spaces defining porosity includes all spaces inside the material that are assessable by another object, such as a molecule, a molecular complex, a particle, or a cell.

The term “crosslinked” or “crosslinking” refers to chemical reactions joining two polymers or nano- or microstructures, or two parts of a same polymer or nano- or microstructures, together through bonds that may be covalent, electrostatic, or physical interactions.

The term “stable” or “stability” as used herein in connection with a hydrogel or a polymeric material includes thermal stability over a prolonged period of time (e.g., at least 3 days) without significant (e.g., 5% or more) decomposition or loss of mechanical integrity. The term stability can also include a continued integrity at the molecular and microscale of a microparticle- or fiber-based material or system in an uncrosslinked or crosslinked state during it's designed shelf life. The shelf life of such material or system may be suitable for its intended use. For example, the material or system may be designed to degrade on a shorter time scales (e.g., a few hours to a day) for cell-delivery applications, whereas for tissue engineering applications the material or system may be designed to degrade over a longer time scales (e.g., at least a day).

The present disclosure relates to polymeric materials having desirable stability and other mechanical properties at relatively high porosity, which may be useful for a wide range of application including 3D bioprinting, regenerative medicine, and tissue engineering. Porosity in granular hydrogel scaffolds is typically increased upon decreasing the packing density. This approach is limited as low packing density reduces the inter-granule contacts that are required to create surface-surface bonds, which are critical in stabilizing the final scaffolds. While literature does not report the upper limit for porosity achieved in this fashion, porosity higher than 30-40% have not been reported using this approach, and none have been cultured for longer than 7 days. Further, in vivo studies have not reported granular hydrogel scaffolds with porosity higher than 20% but have shown beneficial effects toward cellular infiltration as porosity and pore size increased. There is a need for structurally more permissive materials, such as materials which have large amounts of porosity and/or contain components that can move past one another within a stable bulk material.

The present disclosure addresses this need. In various embodiments, the present disclosure provides long polymeric fibers that entangle or span across gaps and multiple particles, resulting in improved stability. Remarkably, the present disclosure demonstrates that such mechanical stability may not need covalent crosslinking between fibers and other fibers or between fibers and microparticle components. In certain embodiments, cells can be incorporated into these permissive materials and move within them. For example, the present materials can be stretched and the individual fibers can slide past one another. In certain embodiments, the present disclosure provides stable extruded materials (including 3D printed filaments) that consist entirely and only of fibers that are not crosslinked to one another. These materials can be permissive both through porosity and ability to move, which is desirable in many applications. As nonlimiting illustration, an extruded or injected material should maintain stability at a deposited location. While the material can be crosslinked (e.g., by photocrosslinking or exposure to enzymatic activity or other physical stimuli such as temperature changes or sonication), there might be instances where crosslinking is not possible and another mechanism needs to be employed to stabilize the material. Advantageously, the present disclosure can control porosity in fibrous systems (e.g., a system with removable particles or an all-fiber system) while maintaining stability, even without the need to crosslink the polymeric fibers. In some embodiments, the present disclosure provides a fibrous system in which some fractions of fibers and/or microparticles are crosslinked to one another and other fractions are not crosslinked, which can provide unique mechanical properties such as stress-relaxation and toughness.

In one aspect, the present disclosure provides a scaffold comprising polymeric fibers having an aspect ratio of at least 20 and hydrogel microparticles, wherein the polymeric fibers and the hydrogel microparticles form a stable network. The aspect ratio of the polymeric fibers can be at least 25, at least 30, at least 40, at least 50, at least 60, at least 70, at least 80, at least 90, at least 100, at least 150, at least 200, at least 250, or at least 300. Each fiber can be in contact with, or attached to, another fiber or a plurality of the HMPs along the length of the fiber or at discrete locations along the fiber, thereby forming a stable network through long range interactions between the fibers and/or between the fiber and the hydrogel microparticles. In contrast, only interactions between neighboring particles are available in a composition containing only spherical particles. The long-range interactions as described herein can involve any kind of physical attachment or chemical bonding. As nonlimited examples, the interaction can include covalent bonding and non-covalent interactions. Suitable covalent bonding includes, for example, crosslinking (e.g., light triggered), click-type chemistries, Michael additions, pH-driven interactions, amine-aldehyde (hydrazone) binding. Suitable non-covalent interactions include, for example, supramolecular chemistry, hydrogen bonds, molecular entanglements via molecules grafted on the surface, DNA-based interactions (e.g., through the grafting on complementary single-stranded DNAs to surfaces), and hydrophobic binding.

In some embodiments, the scaffold has a porosity of at least 30%. The porosity is at least 30%, at least 40%, at least 50%, at least 60%, at least 70%, at least 80%, or at least 90%. Remarkably, the scaffold can be stable and have desirable mechanical properties at a porosity of at least 30%.

In some embodiments, the polymeric fibers of the scaffold are crosslinked and/or the hydrogel microparticles are crosslinked. For example, a fiber polymer molecule can be crosslinked with one or more other fiber polymer molecules, a fiber polymer molecule can be crosslinked with one or more hydrogel microparticles, or a hydrogel microparticle can be crosslinked with one or more other hydrogel microparticles. The crosslinking may improve the stability of the scaffold or adjust the mechanical properties of the scaffold. In some embodiments, the crosslinking of the fibers (or hydrogel microparticles) of the scaffold is understood to be the crosslinking between a fiber (or a hydrogel microparticle) with another fiber or a hydrogel microparticle, which is separate and different from the process of making the fibers (or hydrogel microparticles) involving crosslinking polymer molecules as building blocks of the fibers (or hydrogel microparticles).

In some embodiments, the polymeric fibers of the scaffold are not crosslinked to other components within the scaffold. Without being limited to any theory, it is hypothesized that the long-range interactions offered by the fibers having a high aspect ratio (e.g., at least 25 or at least 100), which include both fiber-fiber interactions and fiber-hydrogel microparticle interactions as described herein contribute to the improved stability of present scaffold, even without crosslinking of the polymeric materials.

In some embodiments, the scaffold further comprises removable particles, which upon removal changes the porosity of the scaffold. For example, the scaffold further comprises removable particles and have a porosity of at least at least 30%, at least 40%, at least 50%, or at least 60%. Upon removal of the removable particles, the porosity can increase to, for example, at least 40%, at least 50%, at least 60%, at least 70%, at least 80%, or at least 90%.

The polymeric fibers as used herein may comprise a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; self-assembled structures including self-assembled peptides or self-assembled peptide amphiphiles; or combinations thereof. In some embodiments, the polymeric fibers comprise hyaluronic acid, poly(ethylene glycol) (PEG), or a combination thereof.

In some embodiments, the polymeric fibers further comprise a crosslinking group or groups. For example, the crosslinking group can comprise a C═C group or a thiol group. In some embodiments, the crosslinking group comprises a photoinitiated crosslinker. In some embodiments, the crosslinking groups comprise norbornene, vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, methacrylate, acrylate, vinyl sulfone, azide, cyclooctyne, hydrazide, aldehyde, thrombin, fibrin, thiols, and combinations thereof. In some embodiments, the crosslinking groups comprise norbornene, methacrylate, thiol, or a combination thereof. Other suitable crosslinking groups known in the art also can be used.

In some embodiments, the polymeric fibers of the scaffold are electrospun fibers. The fibers of the scaffold can be produced by known electrospinning processes. In some embodiments, the polymeric fibers of the scaffold are electrospun fibers having an aspect ratio of at least 30, including but not limited to, an aspect ratio of at least 40, at least 50, and least 80, at least 100, or at least 200.

In some embodiments, the polymeric fibers of the scaffold have a mean diameter of about 0.3 μm to about 7 μm. The mean diameter can be, for example, about 0.3 μm to about 6 μm, about 0.3 μm to about 5 μm, about 0.3 μm to about 4 μm, about 0.3 μm to about 3 μm, about 0.5 μm to about 2.5 μm, about 0.5 μm to about 2.0 μm, or about 0.5 μm to about 1.5 μm. In some embodiments, the mean diameter is about 0.5 μm to about 2.5 μm, including but not limited to, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, and about 2.4 μm.

In some embodiments, the polymeric fibers of the scaffold have a mean length of about 35 μm to about 1 cm. The mean length can be, for example, about 35 μm to about 8 mm, about 35 μm to about 6 mm, about 35 μm to about 4 mm, about 35 μm to about 2 mm, about 35 μm to about 1 mm, about 35 μm to about 800 μm, about 50 μm to about 800 μm, about 50 μm to about 600 μm, about 50 μm to about 400 μm, or about 50 μm to about 200 μm. In some embodiments, the mean length is about 50 μm to about 200 μm, including but not limited to, about 60 μm, about 70 μm, about 80 μm, about 90 μm, about 100 μm, about 120 μm, about 130 μm, about 140 μm, about 150 μm, about 160 μm, about 170 μm, about 180 μm, and about 190 μm.

In some embodiments, the scaffold contains about 5% to about 100% by volume polymeric fibers. The volume percent can be calculated using known techniques and systems, for example, based on the interstitial volume and the packing density of the scaffold. The volume percent of the polymeric fibers can be about 10% to about 80%, such as about 10%, about 20%, about 30%, about 40%, about 50%, about 60%, about 70%, or about 80%. In some embodiments, a “fully jammed” material, with approximately 100% fibers by volume can be produced by high speed centrifugation (˜20,000+rcf). Addition of liquid or a removable particle (e.g., a particle that is removed by thermal melting) can lower the vol % of the fibers. For example, an 80 vol % fiber system can include, by volume, 8 parts of fibers and 2 parts of medium; or 8 parts of fibers, 1 part of gelatin particles, and 1 part of medium; or 8 parts of fibers and 2 parts of particles. It is understood that some degree of porosity would remain between the fibers, as the particles theoretically could not perfectly fill all voids. In other words, actual void space between particles would not quite reach 0% as the fiber approaches 100% by volume in the material.

In some embodiments, the hydrogel microparticles of the scaffold comprises a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile; or combinations thereof. In some embodiments, the hydrogel microparticles comprises hyaluronic acid, poly(ethylene glycol), or a combination thereof.

In some embodiments, the hydrogel microparticles of the scaffold further comprise a crosslinking group. In some embodiments, the crosslinking group comprises a C═C group or a thiol group. The crosslinking group may further comprise a photoinitiated crosslinker or a crosslinker responsive to exposure to enzymatic activity or other physical stimuli such as temperature changes or sonication.

In some embodiments, the crosslinking group of the hydrogel microparticles is selected from norbornene, methacrylate, acrylate, vinyl sulfone, azide, cyclooctyne, hydrazide, aldehyde, thrombin, fibrin, or combination thereof. In some embodiments, the crosslinking group of the hydrogel microparticles comprises norbornene, methacrylate, or a combination thereof.

In some embodiments, the removable particles comprise gelatin, extracellular matrix-derived hydrogel particles (including Matrigel), poly alpha esters, poly ester amides, particles formed from hydrogels with enzymatically cleavable crosslinks, particles from hydrogels with physical crosslinks, alginate particles, agarose particles, pluronic, poly-NIPAAM-based particles, or combination thereof. In some embodiments, the removable particles comprise gelatin.

In another aspect, the present disclosure provides a method of producing a stable scaffold. The method comprises mixing polymeric fibers having an aspect ratio of at least 20 and hydrogel microparticles, thereby the polymeric fibers and the hydrogel microparticles form a stable network. In some embodiments, the polymeric fibers have an aspect ratio of at least 25, such as at least 30, at least 50, at least 100, at least 150, or at least 200.

In some embodiments, the method further comprises crosslinking the polymeric fibers and/or crosslinking the hydrogel microparticles. The polymeric fibers and the hydrogel microparticles can each comprise a crosslinking group as described herein. For example, a polymeric fiber can be crosslinked with one or more other polymeric fibers or one or more hydrogel microparticles, and/or a hydrogel microparticle can be crosslinked with one or more polymeric fibers or one or more other hydrogel microparticles. In some embodiments, the polymeric fibers comprise hyaluronic acid, poly(ethylene glycol) (PEG), or a combination thereof. In some embodiments, they polymeric fibers further comprise a crosslinking group, such as norbornene, methacrylate, thiol, or a combination thereof. In some embodiments, the hydrogel microparticles comprises hyaluronic acid, poly(ethylene glycol) (PEG), or a combination thereof. In some embodiments, the hydrogel microparticles further comprise a crosslinking group, such as norbornene, methacrylate, or a combination thereof.

In some embodiments, the scaffold comprises removable particles and the method further includes removing the removable particles, thereby changes the porosity of the scaffold. For example, the polymeric fibers, the hydrogel microparticles, and the removable particles can be mixed to form a stable structure. Removal of the particles can increase the void space between the polymeric fibers and the hydrogel microparticles, thereby increasing the porosity of the scaffold. The particles can be removed by known procedures, such as thermal melting, backwash, enzymatic degradation, hydrolysis, chemical or physical disruption of physical bonds (e.g., through solvents, changes in ionic charges, changes in hydrophobicity/hydrophilicity within the removable particles, the use of solutes with affinity for components of the crosslinking scheme) within the removable particles, and any other form or removal procedure of sacrificial particles.

In another aspect, the present disclosure provides a method of culturing cells. The method comprises, mixing the cells with the scaffold as described herein thereby the cells are embedded in the scaffold, and culturing the cells embedded in the scaffold. In some embodiments, the scaffold comprises polymeric fibers having an aspect ratio of at least 25 (such as at least 30, at least 50, at least 100, or at least 200) and hydrogel microparticles, in which the polymeric fibers and the hydrogel microparticles form a stable network. In some embodiments, the cell comprises cells critical for vasculature formation including human umbilical vein endothelial, murine myoblasts (C2C12 s, ATCC, etc), fibroblasts, induced pluripotent stem cells, mesenchymal stromal cells, neural stem cells, hepatoctyes, macrophages, beta cells and pancreatic islets, cells of the immune system, cells of any tissue or organ system in the body, cells derived from diseased tissue including cancer and tumor cells, genetically modified cells, and any other cell that can be cultured in 2D or isolated from an organism for 3D culture.

The stability and mechanical properties of the scaffold can be adjusted by crosslinking. However, due to the long-range interactions of the polymeric fibers as described herein, crosslinking of the scaffold may not be required for the present method. The degree of crosslinking may be controlled using known techniques to adjust the stability of the scaffold for specific applications in cell culture and tissue engineering. Crosslinking, if desired, can be carried out before or after mixing the cells with the scaffold. In some embodiments, the present method of culturing cells further comprises, following mixing cells with the scaffold, crosslinking the polymeric fibers and/or crosslinking the hydrogel microparticles of the scaffold.

The use of the present scaffold for applications in regenerative medicine and tissue engineering may involve known bioprinting techniques. In some embodiments, the present method may further comprise, prior to culturing the cells embedded in the scaffold, extruding the scaffold having embedded cells. In some embodiments, the scaffold with embedded cells has suitable mechanical properties to form an extrudable composition, such as a printable or injectable composition. As a nonlimiting example, the scaffold having embedded cells can be extruded by 3D printing. Suitable 3D printing systems (including printers and software) include those known in applications of bioprinting and tissue engineering. In some embodiments, the scaffold is used as a support material (also known as a support bath) into which materials and cells might be printed (in 3D printing approaches known as embedded printing). In some embodiments, the scaffold as a support material includes cells, fibers, microparticles, and removable particles in various ratios, as desired. In some embodiments, the present scaffold is used as both a support material and an extruded material.

Cell growth and cell behavior can be affected by the porosity of the scaffold as described herein, which in turn can be controlled by inclusion of removable particles. Removal of such particles can increase the porosity of scaffold, thereby changing the microenvironment in which the cells are cultured. In some embodiments, the scaffold comprises removable particles and the method further comprises, prior to mixing the cells with the scaffold, removing the removable particles, thereby changing the porosity of the scaffold.

In another aspect, the present disclosure provides a hydrogel comprising polymeric fibers having a mean length of at least 35 μm, wherein the hydrogel is stable and has a porosity of at least 30%.

The mean length of the polymeric fibers of the hydrogel can be at least 35 μm to about 1 cm, such as about 40 μm to about 8 mm, about 40 μm to about 6 mm, about 40 μm to about 4 mm, about 40 μm to about 2 mm, about 40 μm to about 1 mm, about 45 μm to about 800 μm, about 50 μm to about 800 μm, about 50 μm to about 600 μm, about 50 μm to about 400 μm, or about 50 μm to about 200 μm. In some embodiments, the mean length of the polymeric fibers in the hydrogel is about 50 μm to about 400 μm, including but not limited to, about 60 μm, about 80 μm, about 100 μm, about 120 μm, about 140 μm, about 160 μm, about 180 μm, about 200 μm, about 220 μm, about 240 μm, about 260 μm, about 280 μm, about 300 μm, about 320 μm, about 340 μm, about 360 μm, and about 380 μm.

The porosity of the hydrogel can be at least 30%, at least 40%, at least 50%, at least 60%, at least 70%, at least 80%, or at least 90%. The hydrogel can maintain stability and desirable mechanical properties at a porosity of at least 30%. The porosity of the hydrogel can be adjusted by inclusion of removable particles. For example, the hydrogel further comprises removable particles and have a porosity of at least at least 30%, at least 40%, at least 50%, or at least 60%. Upon removal of the removable particles, the porosity of the hydrogel can increase to, for example, at least 40%, at least 50%, at least 60%, at least 70%, at least 80%, or at least 90%.

In some embodiments, the hydrogel comprises the polymeric fibers as the only polymer component (e.g., an all-fiber system). In some embodiments, the hydrogel further comprises hydrogel microparticles, wherein the polymeric fibers and the hydrogel microparticles form a stable network (e.g., a fiber-microparticle system). The polymeric fibers and the hydrogel microparticles can form a reinforced structure with improved stability and mechanical properties, compared to a system that includes the polymeric fibers alone or the hydrogel microparticles alone, particularly where the hydrogel has high porosity (e.g., at least 30%).

In some embodiments, the polymeric fibers of the hydrogel comprise a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile; or combinations thereof. In some embodiments, the polymeric fibers of the hydrogel comprise hyaluronic acid, poly(ethylene glycol), or a combination thereof.

In some embodiments, the polymeric fibers of the hydrogel are not crosslinked. While the fiber may be produced in a process (e.g., electrospinning) that involves crosslinking polymer molecules as building blocks, the individual fibers may not need further crosslinking between themselves to stabilize the hydrogel. In some embodiments, the polymeric fibers are not crosslinked between themselves, and the hydrogel remains stable. Without being limited to any theory, it is hypothesized that, due to the length of the fibers, the long range interactions other than chemical crosslinking between the fibers (such as entanglement, hydrogen bonding, or hydrophobic interactions) can be sufficient to stabilize the hydrogel, even if the hydrogel has a diluted (low) concentration of the fibers and high porosity (e.g., at least 30%). Remarkably, the present hydrogel with non-crosslinked polymeric fibers offers unique advantage over the existing fibrous hydrogel systems in many applications (such as 3D printing). For cell culture and tissue engineering applications, the present fibrous system, stabilized without crosslinking, can be loaded with cells, move, and yield to cell behaviors. In addition, crosslinking can be designed to create partial or full populations of crosslinked fibers and particles, allowing fine tuning of the properties of the present fibrous system according to the specific application.

In some embodiments, the polymeric fibers of the hydrogel further comprise a crosslinking group. In some embodiments, the crosslinking group is norbornene, methacrylate, acrylate, vinyl sulfone, azide, cyclooctyne, hydrazide, aldehyde, thrombin, fibrin, or a combination thereof. In some embodiments, the crosslinking group is norbornene, methacrylate, or a combination thereof.

In some embodiments, the polymeric fibers of the hydrogel are electrospun fibers. The polymeric fibers can be prepared, for example, by known electrospinning techniques.

In some embodiments, the polymeric fibers of the hydrogel have an aspect ratio of at least 15. The aspect ratio of the polymeric fibers of the hydrogel can be at least 20, at least 25, at least 30, at least 50, at least 100, at least 200, or at least 300. In some embodiments, the polymeric fibers of the hydrogel have an aspect ratio of at least 25.

In some embodiments, the polymeric fibers of the hydrogel have a mean diameter of about 0.3 μm to about 7 μm. The mean diameter can be, for example, about 0.3 μm to about 6 μm, about 0.3 μm to about 5 μm, about 0.3 μm to about 4 μm, about 0.3 μm to about 3 μm, about 0.5 μm to about 2.5 μm, about 0.5 μm to about 2.0 μm, or about 0.5 μm to about 1.5 μm. In some embodiments, the mean diameter of the polymeric fibers of the hydrogel is about 0.5 μm to about 2.5 μm, including but not limited to, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, and about 2.4 μm.

In some embodiments, the hydrogel includes about 10% to about 80% by volume the polymeric fibers. The volume percent of the polymeric fibers of the hydrogel can be, for example, about 10%, about 20%, about 30%, about 40%, about 50%, about 60%, about 70%, or about 80%.

In some embodiments, the present disclosure provides a scaffold comprises a stable hydrogel as described herein. In some embodiments, the hydrogel is a stable hydrogel having a porosity of at least 30% and comprises polymeric fibers having a mean length of at least 35 μm.

Due to its unique mechanical properties and stability, the hydrogel may be used in a wide range of applications, including, but not limited to, 3D printing, tissue engineering, and applications in regenerative medicine, drug delivery, and implantation. In some embodiments, the present disclosure provides a support material for 3D printing, which comprises the hydrogel as described herein. In some embodiments, the present disclosure provides an injectable or extrudable composition comprising the hydrogel as described herein. In some embodiments, the present disclosure provides a scaffold comprising the hydrogel as described herein. In some embodiments, the present disclosure provides an ink or filament for 3D printing, which comprises a stable hydrogel as described herein. The ink or filament can be used for bioprinting applications. In some embodiments, the ink or filament or support material comprises cells embedded in the hydrogel. In some embodiments, a scaffold for cell culture is produced by printing the ink or filament using a 3D printer.

In another aspect, the present invention discloses a method of preparing a hydrogel. The method comprises electrospinning a polymer into a polymeric fiber having a mean length of at least 35 μm and hydrating the polymeric fiber in an aqueous medium to form a stable hydrogel having a porosity of at least 30%.

In some embodiments, the method comprises electrospinning a polymer into a polymeric fiber and segmenting the polymeric fiber, the segmented polymeric fiber having a mean length of at least 35 μm. The length of the fibers may be controlled, for example, by segmenting the fiber produced by electrospinning. Segmentation may be carried out by any suitable technique. For example, the fibers may be segmented by a series of trituration steps through needles (e.g., a series of 16 g, 18 g, and finally 20 g needles). Alternatively, a controlled UV crosslinking (e.g., crosslinking only fibers in fields of lengths defined by a photomask) of fibers and physical cutting may be used to create controlled fibers lengths.

The preparation method can include known electrospinning procedures and reagents. As a non-limiting example, the polymers (e.g., norbornene-containing polymers, thiol-containing polymers, polyethylene oxide) can be suspended in an aqueous solution, and the solution was electrospun to produce a polymeric fiber. The preparation method can further comprise centrifugation to isolate the electrospun fiber. In some embodiments, at least 50% v/v of the electrospun fiber is recovered from centrifugation. In some embodiments, addition of liquid or a removable particle (e.g., a particle that is removed by thermal melting) can lower the vol % of the fibers. For example, an 80 vol % fiber system can include, by volume, 8 parts of fibers and 2 parts of medium; or 8 parts of fibers, 1 part of gelatin particles, and 1 part of medium; or 8 parts of fibers and 2 parts of particles. It is understood that some degree of porosity would remain between the fibers, as the particles theoretically could not perfectly fill all voids. In other words, actual void space between particles would not quite reach 0% as the fiber approaches 100% by volume in the material. In some embodiments, the hydrogel produced by the present method has a modulus of at least 120 Pa, at least 90 Pa, or at least 50 Pa at strains below a yield strain. In some embodiments, the hydrogel produced by the present method has a yield strain of about 50% or higher. In some embodiments, the yield strain is about 140%. In some embodiments, the hydrogel formed from the fiber particles converts from solid-like to liquid-like behaviors and back in 1-20 s when alternating between strains in excess and below the yield strain. In some embodiments, the fiber particle-based hydrogels can be stretched 0-2500% before failure. In some embodiments, fibers of different lengths, physical properties, or chemical composition might be combined to modulate mechanical properties. In some embodiments, the storage moduli of the fibers are ˜10× the loss moduli. In some embodiments, the fiber-based granular hydrogels were able to dissipate stress within T1/2 of >10 s and of >100 s, under a constant applied shear strain of 15%.

In another aspect, the present disclosure provides a process of 3D printing. The process can include printing an ink or filament comprising a stable hydrogel as described herein using a 3D printer.

In another aspect, the present disclosure provides a method of culturing cells. The method can include mixing the cells with a scaffold comprising the stable hydrogel as described herein or a 3D printed scaffold as described herein, thereby the cells are embedded in the scaffold. The method can further comprise culturing the cells embedded in the scaffold.

In another aspect, the present disclosure provides another method of culturing cells. The method can include printing an ink or filament comprising the stable hydrogel as described herein into a scaffold using a 3D printer, the hydrogel having cells embedded therein, and culturing the cells embedded in the scaffold.

### Examples

### Example 1

Scaffolds composed of crosslinked hydrogel microparticles (HMPs), or granular hydrogel scaffolds, contain pore spaces much greater in size than conventional bulk hydrogels. Packing of HMPs creates interconnected, micron-scaled openings that are preserved upon crosslinking. Packed HMP scaffolds, also known as microporous annealed particle (MAP) scaffolds, greatly improve cell migration and proliferation. However, while higher porosity allows for more cellular infiltration, it also reduces the mechanical integrity of the scaffolds. Here, this challenge is addressed by demonstrating 1) the fabrication of high-porosity granular scaffolds beyond what is possible from particle packing using sacrificial HMPs; and 2) stabilization of the high-porosity scaffolds by incorporating electrospun hydrogel fibers within the scaffolds. granular scaffolds were fabricated using norbornene-modified hyaluronic acid (norHA) HMPs and gelatin HMPs as the sacrificial population. HMP scaffolds were created with up to 50% porosity by incorporating (0-50 vol. %) sacrificial gelatin HMPs. When electrospun norHA fibers were incorporated in scaffolds at 5-10 vol. %, highly porous scaffolds retained their structure over a period of 28 days. Additionally, HUVECs were mixed with HMPs prior to scaffold formation and exhibited high viability. The HUVEC-HMP mixture could be injected through a needle and then crosslinked to form a scaffold, with post-injection viability >80%. Overall, this study demonstrates the development and characterization of a stable, highly porous granular scaffold system with high processability and cytocompatibility.

### 1 Introduction

Hydrogels have been widely used as 3D cell culture platforms. Synthetic and natural polymers have been engineered to recapitulate characteristics of the extracellular matrix (ECM), including mechanical properties, degradability, and bioactive ligands that guide cell fate. Among the various strategies of hydrogel engineering, fabricating hydrogels as microparticles has become a popular approach. Hydrogel microparticles (HMPs) can be packed together to create a bulk material that can transition from a solid-like state to a liquid-like state: when the applied stress is less than the yield stress of the packed HMPs, the bulk material responds elastically to deformation. However, once applied stress surpass the yield stress, the HMPs can flow pass each other and the bulk material exhibits shear-thinning behavior. This property is particularly desirable for injection-based applications and 3D printing. Moreover, even when fully packed, interconnected, micron-scale porosity exists among the HMPs, priming the system for easy cellular infiltration. This porosity is preserved upon crosslinking HMPs into a scaffold, as shown extensively with microporous annealed particle scaffolds (or MAP scaffolds). HMPs in MAP scaffolds can be crosslinked after injection into wound sites and greatly enhance tissue regeneration.

While the micro-scale porosity in packed HMPs is desirable for cellular infiltration, porosity comes at the cost of mechanical integrity. In bulk hydrogel scaffolds, polymer chains crosslink to form nanoscale meshes, creating a highly crosslinked network to hold the scaffold together. In HMP scaffolds, while the HMPs themselves possess the same polymer network, the crosslinks that work to hold the scaffold together only exist at HMP interfaces. The number of existing crosslinks could be magnitudes lower and farther apart in HMP scaffolds, resulting in a weaker bulk scaffold. Currently, increasing the stability of HMP scaffolds have not been thoroughly investigated. Some stabilization approaches include using crosslinked interstitial matrix or interpenetrating networks throughout the HMP scaffold. However, both methods require a polymer network to be formed within the interstitial space, doing away with the micro-scale interstitial porosity and focusing on applications that do not require a microporous scaffold. Other potential approaches to increase stability include decreasing HMP sizes and increasing packing density. Both serve to create more interacting surfaces, or crosslinking nodes, among the HMPs. However, as smaller HMPs pack more densely, the interstitial pore sizes also become smaller and less accessible to cells; and increases in packing density similarly affect porosity.

Here, another approach to increase HMP scaffold stability that does not impinge on HMP packing and scaffold porosity is explored. The stabilization of HMP scaffolds by introducing fiber-mediated long-range interactions between HMPs. In bulk hydrogel, polymer chains form a covalent network, a singular chain cannot be perturbed without perturbing the entire network. In HMP scaffolds, however, individual HMPs only interact with their immediate neighbors. When the force of local perturbations exceeds the strength of interfacial associations, the perturbed HMPs could easily dissociate from the system. Here, to extend interactions between HMPs beyond their immediate surroundings, electrospun fibers were incorporated into HMP scaffolds. By adding single-micron diameter fibers with lengths greater than HMP diameters, multiple layers of HMPs can associate through their interactions with fibers. Additionally, fiber stabilization allows for the establishment of highly porous scaffold with long-term stability. High porosity was created by addition of sacrificial HMPs into the scaffold and demonstrate the ability to control pore fraction and pore sizes. Finally, this work demonstrates that packed HMPs can be injected with cells for facile cell culture, as well as potential applications in cell delivery and regenerative medicine.

### 2 Results and Discussion

2.1. Fabrication and Characterization of norHA HMPs

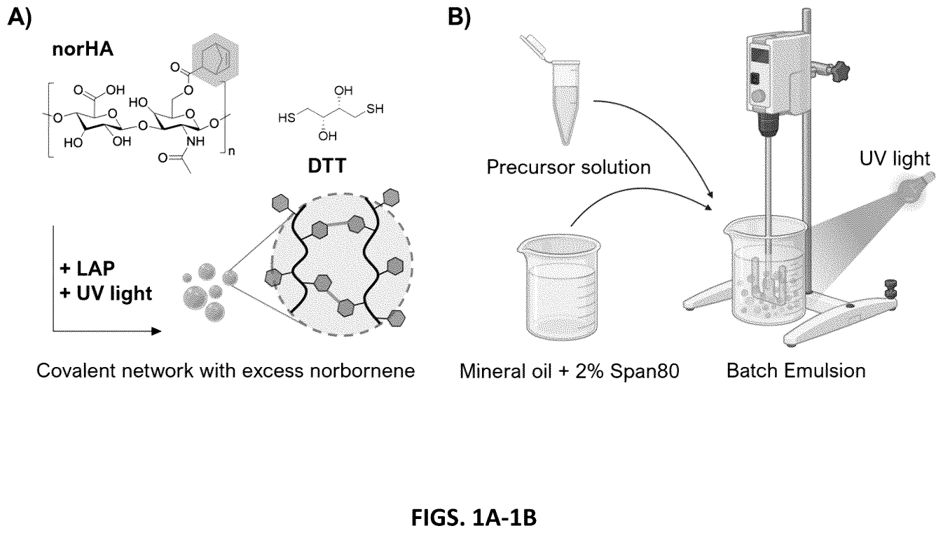

HMPs were generated from norbornene-modified hyaluronic acid (norHA). Hyaluronic acid (HA) is a naturally derived polysaccharide that is biocompatible, biodegradable, and amenable to chemical modification. The addition of norbornene pendant groups to HA enables reactions of norHA to thiolated molecules: a highly efficient thiol-ene click chemistry that allows for stoichiometric control over the consumption of norbornene groups by limiting the available thiol-containing groups (FIG. 1A). This enables the preservation of norbornene pendant groups for secondary conjugations. Here, norHA was synthesized with a 0.3 degree of substitution (DoS), confirmed by ¹H NMR. By controlling the thiolated crosslinker to norbornene concentration, 0.3 DoS sufficiently support both HMP-forming crosslinking and subsequent crosslinking among formed HMPs (Table 1).

TABLE 1
Varying norHA compositions and available reactive groups.

| | Soft | Medium | Stiff |
|---|---|---|---|
| norHA (wt %) | 2 | 3 | 4 |
| DTT (mM) | 2 | 3 | 4 |
| total norbornene (mM) | 15 | 22.5 | 30 |
| remaining norbornene (mM) | 11 | 16.5 | 22 |
| % norbornene consumed | 26.7 | 26.7 | 26.7 |
| | | norHA DoS: | 0.3 |

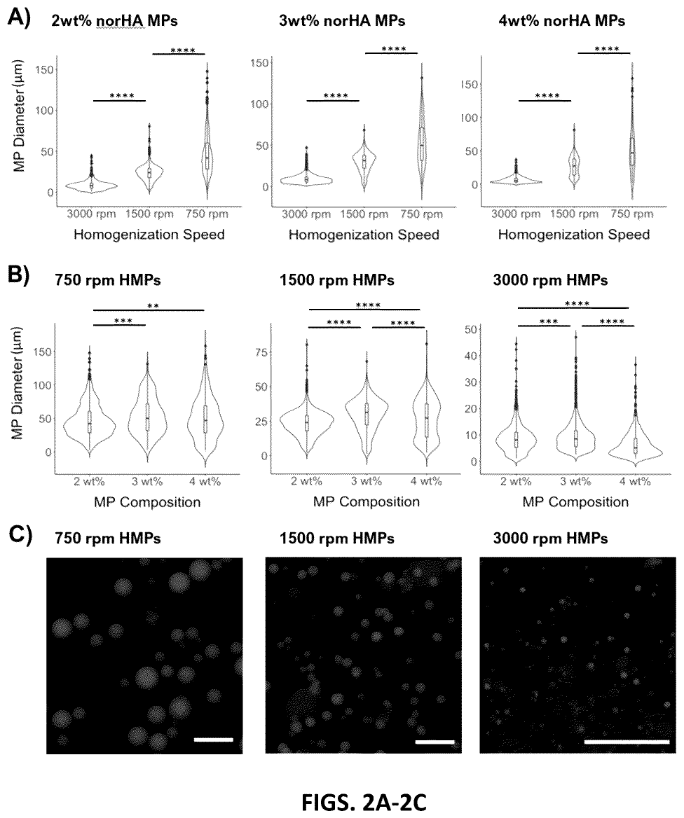

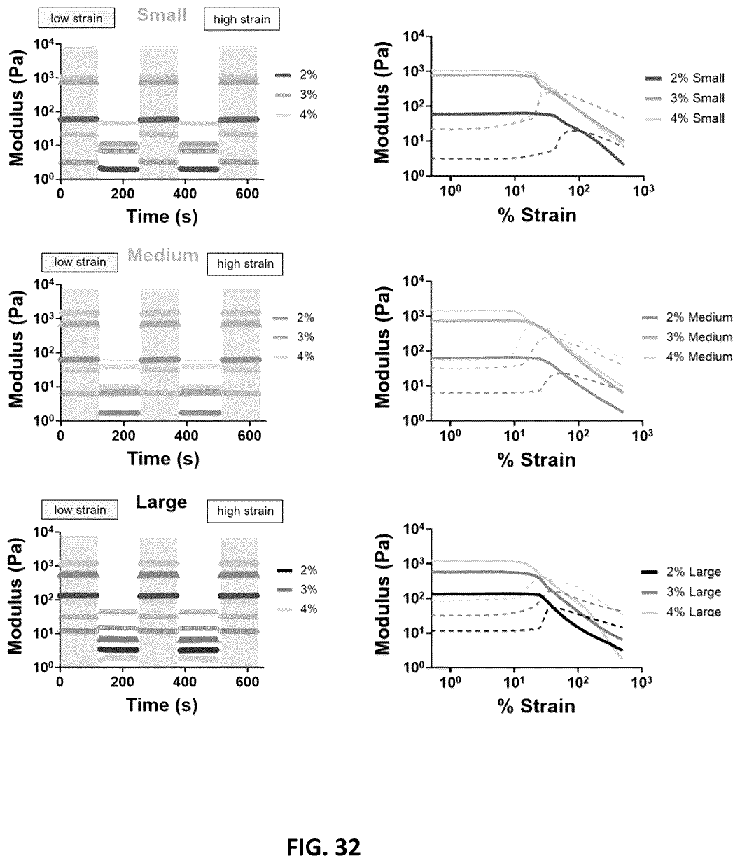

Using batch emulsification, norHA HMPs were generated (FIG. 1B) with varying moduli and size by varying the HMP composition and the speed of homogenization, respectively. Soft, medium, and stiff HMPs are made using 2% w/v, 3% w/v, and 4% w/v norHA concentration and 2 mM, 3 mM, and 4 mM DTT concentration, respectively (Table 1). The mechanical properties of the HMPs are characterized by their storage moduli using oscillatory rheology. Soft, medium, and stiff HMPs have average storage moduli of 782 Pa, 1758 Pa, and 3797 Pa, respectively (FIG. 32). The size of norHA HMPs can also be altered by changing the spin speed of the homogenizer used for emulsification. Small, medium, and large HMPs were generated at 750 rpm, 1500 rpm, and 3000 rpm. For each modulus, HMPs with mean diameter ranging from <10 μm to 50 μm were generated (Table 2). Due to the nature of the emulsion process, size distributions of HMP populations were polydisperse. Polydispersity increased as the stir speed decreased (FIG. 2A); however, mean sizes were well-separated and statistically significant for groups across stir speeds. Effect sizes were also calculated for each comparison. The smallest effect size calculated for comparing between stir speed was 1.17, accounting for a large effect (>0.8). In comparing the HMP size distribution across HMP composition; however, the largest effect size calculated was 0.55, accounting for a medium effect (>0.5), with most groups having a small effect (>0.2) and less (FIG. 2B).

TABLE 2
NorHA HMP mean sizes for all compositions and speeds.

Mean Diameter (μm)

| HMP composition | 750 rpm | 1500 rpm | 3000 rpm |
|---|---|---|---|
| 2 wt % norHA | 46 ± 23.5 | 23.2 ± 9.53 | 8.65 ± 5.24 |
| 3 wt % norHA | 52.7 ± 27 | 29.5 ± 11.6 | 9.33 ± 5.24 |
| 4 wt % norHA | 49.7 ± 30.9 | 26.6 ± 14 | 6.49 ± 5.02 |

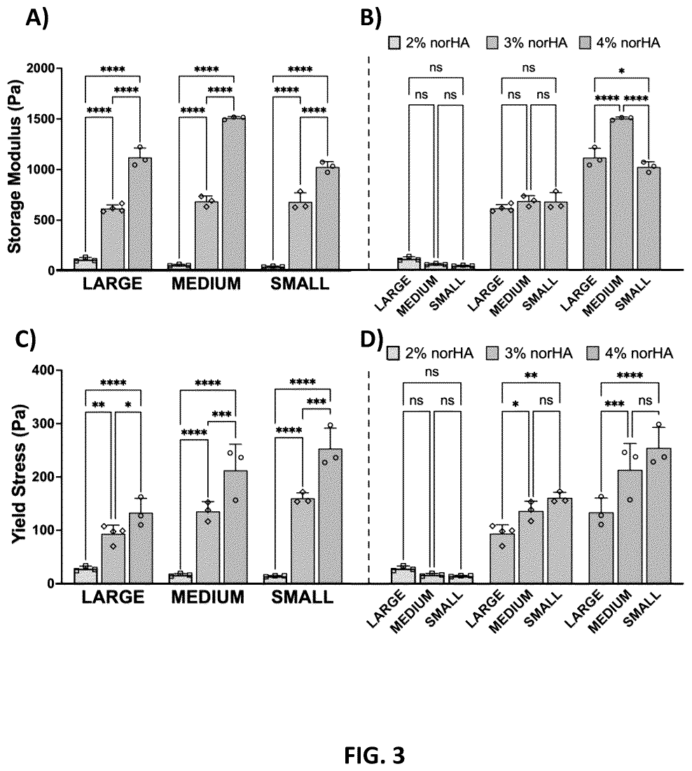

Next, rheological tests were conducted to investigate the flow properties of packed HMPs. Packed HMPs have been well documented to be able to flow under external forces and recover when applied force is removed. This property renders HMPs injectable and enabled various applications of HMPs in 3D printing and injectable therapeutics. These HMPs were confirmed to exhibit the same behavior using time sweeps alternating between 0.5% and 500% strain. Frequency and strain sweeps were also conducted for all HMPs. HMP modulus observed changes were correlated to changes in granular hydrogel moduli. When HMPs are of the same size, increases in HMP modulus resulted in increases in bulk modulus (FIGS. 3A-3D). However, changes in HMP sizes did not affect the bulk modulus except the 4% norHA group (FIGS. 3A-3D).

Another important metric to HMP processability is the resistance to flow by packed HMPs, often described by yield stress of the system. Here, yield stress was measured using oscillatory strain sweeps supplemented by shear rate sweeps focused on lower shear rates (FIG. 3). Similarly to bulk modulus, for HMPs of the same size, yield stress of the packed HMPs positively correlates to changes in individual HMP modulus (FIGS. 3A-3D). Additionally, for 3% norHA and 4% norHA HMPs, yield stress increased as HMPs became smaller (FIGS. 3A-3D). This finding largely corroborates with literature findings of yield strain changes with respect to HMP sizes (Qazi method paper). Yield stress is difficult to define and measure for many material systems (shear band paper). The shear rate sweep profile show that packed HMPs behavior is deviates from traditional yield stress fluid and is reminiscent of shear-banding behavior. This suggests that shear-banding exists within packed HMPs (more shear band paper) and that heterogenous local force distributions contribute to the initiation of flow. A possible explanation to the trend is that larger HMPs, by the nature of their packing, possess less contact density per unit volume compared to smaller HMPs. The lessened contacts may be less suited for dissipating the externally applied force and facilitate the onset of localized disruptions.

The increase in yield stress, or resistance to flow, was also obvious during laboratory processing of the HMPs. When HMPs are transferred by a wide-bore pipette tip, noticeably more resistance and disruptive flow were observed for groups with higher yield stress, especially as HMP modulus increased. Transfer using pipette becomes difficult for groups with yield stress higher than that of 3% norHA, 3000 rpm (small) HMPs. For all subsequent experiments, 3% norHA, 750 rpm (large) HMPs were used due to better optical clarity for larger HMPs, as well as their high processability.

### 2.2. Creating Tunable Porosity in HMP Scaffolds Using Sacrificial Gelatin MPs

Porosity as well as pore sizes in hydrogel scaffolds influence important processes such as cellular infiltration, immune cell responses, and vasculature growth. In HMP scaffolds, particle packing creates interconnected micropores that are challenging to engineer in bulk hydrogels, facilitating cellular infiltration and tissue regeneration when implanted in vivo. Currently, porosity in HMP scaffolds is largely controlled by changing HMP sizes and packing density; however, limitations exist for both. In the case of changing HMP sizes, while increasing the diameter of HMPs increases porosity, the pore sizes also increase uniformly, resulting in larger but fewer pores. HMPs that are too large will also occupy large volumes, making the space less accessible to cells. On the other hand, increasing porosity by decreasing packing density will increase the interstitial fluid volume. Above a certain threshold, cross-linking becomes impossible as interstitial space becomes greater and the HMPs lose physical contact with each other, limiting the porosity.

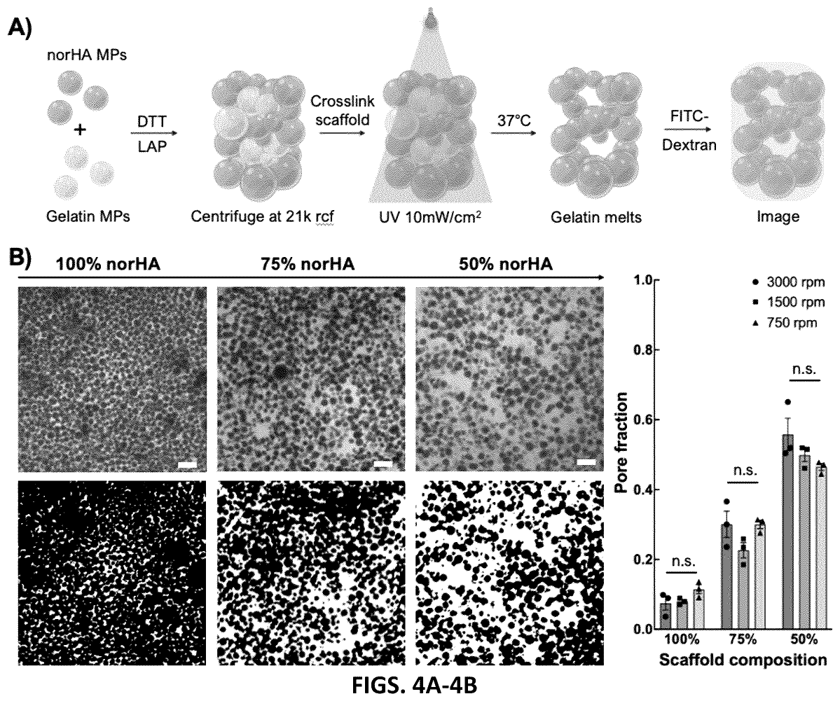

The use of sacrificial HMPs is an alternative method for creating porosity that does not rely on changing particle sizes or packing density. In this study, known volumes of norHA HMPs (scaffold-forming) and gelatin HMPs (sacrificial) were mixed at defined ratios to control porosity in HMP scaffolds. As shown in FIG. 4A, HMP scaffolds were fabricated by UV crosslinking packed HMP precursors containing scaffold-forming HMPs, sacrificial HMPs, and crosslinking agents. Subsequently, sacrificial HMPs were removed and the interstitial solution is replaced with FITC-dextran prior to imaging. Scaffolds with norHA HMPs and gelatin HMPs mixed at 100:0, 75:25, and 50:50 volume ratios were generated for porosity measurement. For each volume ratio group, separate scaffolds were generated using small, medium, and large populations of norHA HMPs to elucidate the effect of size on porosity. For all norHA HMP sizes, scaffolds were consistently generated with ˜10% porosity for 100% norHA scaffolds; ˜30% porosity for 75% norHA scaffolds; and ˜50% porosity for 50% norHA scaffolds (FIG. 4B). However, for each volume ratio group, no statistically significant differences were found for porosity among norHA HMPs with different sizes (FIG. 4B).

A trend of increasing porosity as HMP sizes increased for 100% norHA scaffolds was observed, which corroborates the literature findings. Interestingly, in the small norHA HMPs (3000 rpm, average diameter 6-10 μm) pockets of pore spaces much larger than the HMPs were often observed, whereas pore spaces only existed between packed particles for larger norHA HMPs. This suggests that the centrifugal force was not enough to overcome the microstructures created by small HMPs and may be remedied by more aggressive filtration methods to eliminate interstitial fluid. On the other hand, it is also empirically observed that the small HMPs are more prone to aggregation, suggesting that clustered formations may be energetically favorable as HMP sizes approach the sizes of colloidal particles (ref), at which point thermodynamic forces exert significant influence on particle dynamics. The inherent structure may be desirable for certain applications and has implications on the flow properties of packed HMPs, as they could create local heterogeneity in shear-driven flow that disrupts bulk flow behavior (shear band ref). However, this is outside the scope of the current work.

The effect of gelatin HMP sizes on porosity was also investigated. Similar to norHA HMPs, gelatin HMPs can be made with varying sizes (6-50 μm average diameter) by changing the speed at which they are homogenized. Gelatin HMPs were generated with average diameters less than, equal to, and greater than the average diameters of the norHA HMPs. They were then mixed them together at 75:25 and 50:50 norHA HMP to gelatin HMP volume ratios. Upon confocal imaging, no significant differences among varied gelatin HMP sizes in the same volume ratio group were observed. However, when gelatin HMPs diameters are equal to or greater than that of the norHA HMPs, more disruptive, or discrete pore spaces form. Therefore, for all further experiments, gelatin HMPs with diameters smaller than that of norHA HMPs to eliminate discrete, large pockets of pore space were used.

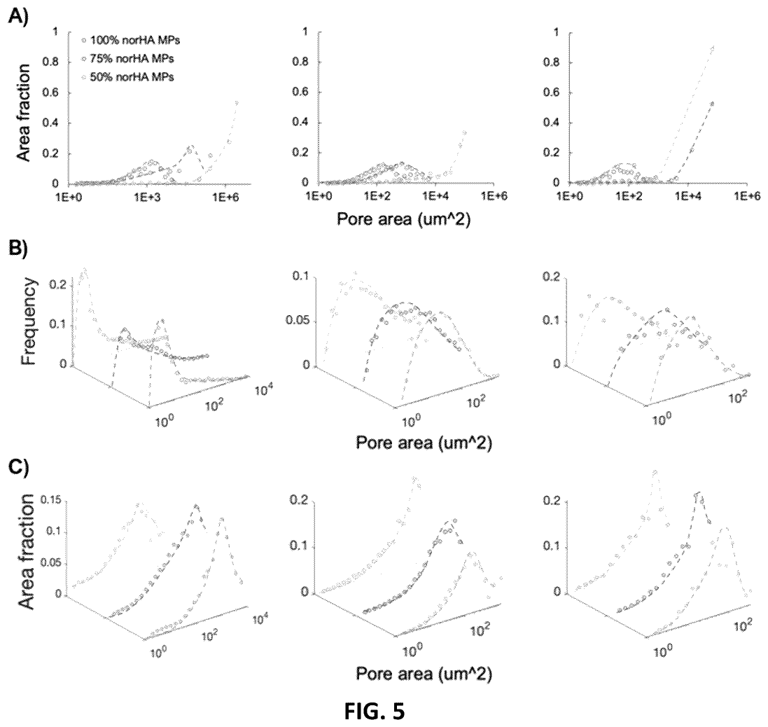

The 2-D pore sizes were quantified using an in-house Matlab program. Briefly, each slice of the confocal images were binarized in FIJI. The binarized image data is then analyzed with a Matlab program that finds inter-connected pixels comprising individual pore spaces. Each pore space recognized by the program was recorded and converted to units of area. The pore area distribution were plotted based on the number of pores (FIG. 5A). Pore distribution based on 2D image analysis revealed that in sample groups with higher porosity, large, open pores occupy most of the pore space but have disproportionately low numbers. To better represent large pores that significantly affect porosity, the pore distribution was plotted based on the area fraction occupied by groups of similar sized pores (FIG. 5A). For each HMP size, larger pore spaces were created as the percentage of sacrificial HMPs increased. When the percentage of sacrificial HMPs reached 50% (25% in the case of 750 rpm norHA), large, interconnected pore spaces are created and accounts for significant portions of the total pore area (FIG. 5A).

Further, in HMP scaffolds with varying porosities, pore size distributions among the small pores were similar. It is hypothesized that these pores existed among packed norHA HMPs. In other words, these pore features exist throughout the packed 100% norHA scaffolds; and in places where packed norHA HMPs were not disrupted by gelatin HMP incorporation. To investigate this, the distribution of only the smaller pores spaces in each group were studied. More specifically, for each norHA HMP size, the largest pore found in the 100% norHA group was used as the cut-off pore size. Pore spaces in 75% norHA and 50% norHA with area larger than the cut-off were eliminated, and the pore size distribution was compared among the groups (FIG. 5B, C). The pore size distribution based on number fraction as well as area fraction showed great consistency among groups with different porosity, suggesting that while higher porosity groups possess much larger pore spaces, the smaller pore spaces that resulted from HMP packing are still present.

### Computational Assessment of Pore Spaces in HMP Scaffolds

Next, to better understand the pore spaces with 3-dimensional analysis a custom computational software was used. To generate scaffold images, a tetrazine-modified fluorophore was used to tag the norHA HMPs. Tetrazine and norbornene undergo additive-free click chemistry and produced a more homogeneous fluorescence compared to thiol-norbornene based fluorophore conjugation. Again, 100% norHA, 75% norHA, and 50% norHA scaffolds were fabricated using large norHA HMPs for custom software analysis. Confocal Z-stack images with 100 μm depth were taken for each scaffold.

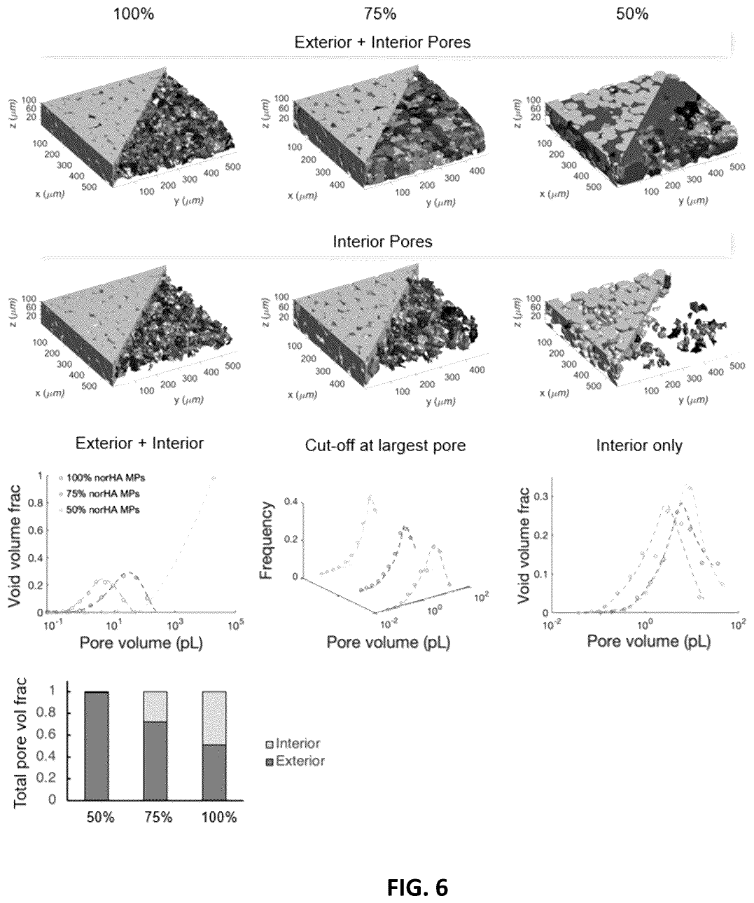

3D analysis with custom software corroborated the trends found in 2D analysis (FIG. 6) Volume fraction distribution (analogous to area fraction described above) of porous scaffold showed similar characteristics to 2D analysis: distributions significantly shift to the right as total porosity in the scaffolds increased. Analysis using largest pore cut-off from 100% norHA group also showed very similar distributions between scaffolds with varying porosity, again corroborating the 2D findings. Further, custom software analysis allowed us to differentiate enclosed, interior pores and open, exterior pores, as illustrated in FIG. 6. Open pores are desirable as they are more accessible for tissue infiltration into the scaffold. It can be visually discerned that as total porosity increased, larger, open pore spaces exist and account for significant portions of the total porosity; however, lower numbers of interior pores exist and account for less of the total porosity. Plotting the volume fraction distribution showed that interior pores of all groups are very similar in size and distribution, but the void spaces occupied by interior pores decreased as porosity increased, with about −50% pore volumes being interior pores for 100% norHA scaffolds to only ˜1% for 50% norHA scaffolds (FIG. 6).

Both 2D and 3D analysis demonstrate the co-existence of large, interconnected pores and small pores in the system. This structure is permissible to engineering multi-scale tissue structures (e.g., vasculature) and may be desirable for many tissue engineering applications. Importantly, porosities in the scaffolds above are achieved predictably, as a direct function of sacrificial HMP fraction. This circumvents the need to dilute packed HMPs or perform other processing steps to reduce packing density. Instead, mixtures of support/norHA and sacrificial/gelatin HMPs can be combined to achieve controlled, high porosity in a single-step process.

### 2.3. Incorporation of Electrospun Fibers Alleviates HMP Scaffold Degradation

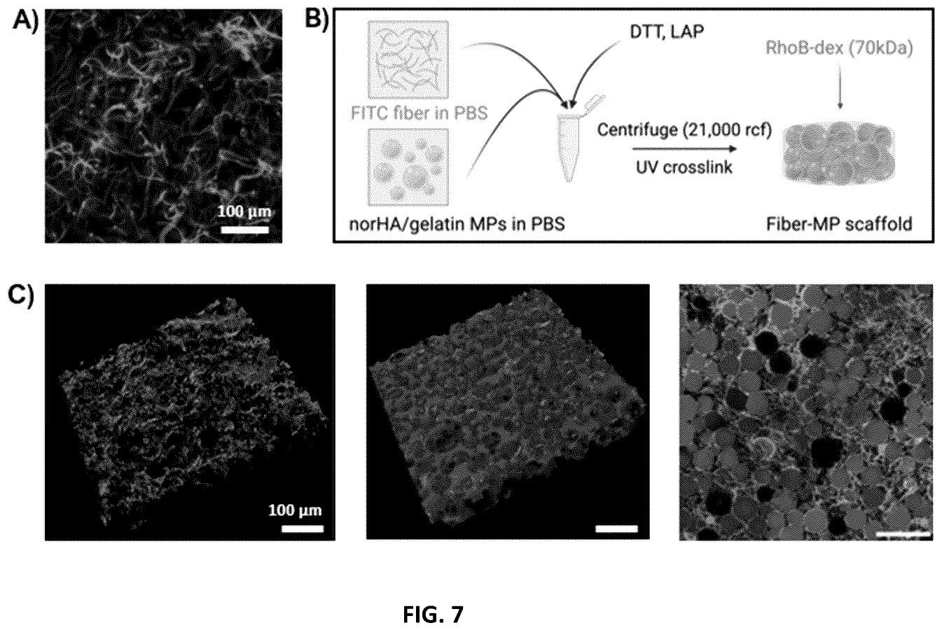

To investigate whether fiber incorporation reinforces HMP scaffolds, initially fibers were created through electrospinning. Electrospun norHA fibers were formed by crosslinking with norbornene-DTT click chemistry. Dry, crosslinked fibers were hydrated in PBS and segmented (FIG. 7A) to yield fibers with high aspect ratio. To visualize the fibers, FITC-dextran was incorporated as an additional inert component in electrospinning. Fluorescent imaging using fibers with FITC-dextran showed the fibers have a mean width of 1.6±0.59 μm and a mean length of 95±48.5 μm. Similar to HMP formation, in crosslinking electrospun norHA fibers, the norbornene to DTT ratio was controlled so that excess norbornene were present after crosslinking. The unreacted norbornenes on fibers undergo secondary crosslinking with the norbornenes on HMP surfaces, strengthening fiber-HMP interactions.

Fibers were incorporated into the HMP scaffolds by mixing all components prior to packing. To form fiber-HMP scaffolds, known volumes of fiber, gelatin HMP, and norHA HMPs were first diluted in PBS for ease of measurement and transfer. Using this method, composite scaffolds were generated with well-defined compositions (FIG. 7B). Confocal imaging of HMP scaffold with fiber incorporation showed that fluorescently labeled fibers distribute within the interstitial space of the HMP scaffolds (FIG. 7C): overlayed images of fluorescent fibers and the interstitial space show that fibers distribute throughout the interstitial space and wrap around the HMPs (spherical-shaped empty spaces) but do not appear inside spaces occupied by HMPs. The norHA HMPs were tagged and observe that fibers were well distributed throughout the space and could bridge between otherwise discrete HMPs.

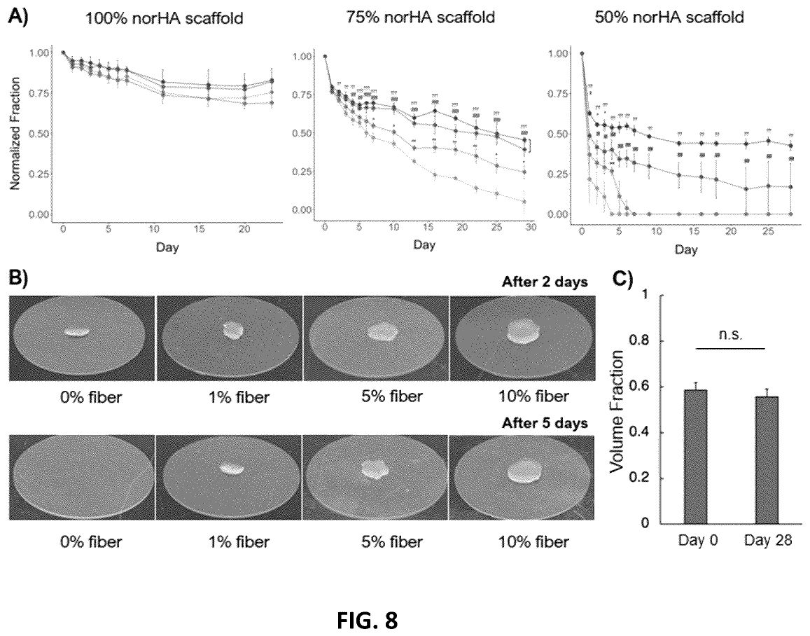

Next, fibers were added to scaffolds composed of 100% norHA HMPs, 75% norHA HMPs, and 50% norHA HMPs, and tracked their degradation over 28 days. For each scaffold composition, the fiber content was varied (0% v/v, 1% v/v, 5% v/v, and 10% v/v) to elucidate the amount of fibers needed to reinforce granular hydrogels in long-term culture. As shown in FIG. 8A, 100% norHA scaffolds retained ˜70% of their initial mass without fiber reinforcement after 28 days of culture in PBS. Incorporation of 1%, 5%, and 10% fiber into 100% norHA scaffolds retained more mass (up to 80%) by the end of culture; however, there were no statistically significant differences, suggesting that these scaffolds are suitable for long term culture without fiber reinforcement. In 75% norHA scaffolds, the incorporation of fibers markedly enhanced the scaffold stability in long-term culture (FIG. 8A). After the initial mass loss between day 0 and day 1 as the result of the removal of sacrificial particles, significant mass loss continued in scaffolds with 0% fiber, presumably through degradation mediated by surface erosion. By day 7, only ˜50% of the scaffold remained for 0% fiber scaffolds. Scaffolds with 1% fiber did not show significant differences compared to 0% fiber scaffolds for the first 6 days. However, from day 7 on, 1% fiber scaffolds degraded significantly less than 0% fiber scaffolds. At day 28, ˜7% of 0% fiber scaffold and ˜32% of 1% fiber scaffold remained. Neither group was able to retain their shape at the end and were not considered suitable for long-term culture. On the other hand, both 5% fiber and 10% fiber scaffolds held their shape over 28 days of study, and both retained >50% of their initial weight. Additionally, at no point of the study were the two groups statistically different from each other, indicating that both 5% fiber and 10% fiber scaffolds were suitable candidates for long-term culture. Finally, in 50% norHA scaffolds, the reinforcement effect of fibers was the most pronounced (FIG. 8A, B). Without the incorporation of fibers, 50% norHA scaffolds dissociated starting from day 3, breaking apart into smaller pieces and losing most of its weight. Adding 1% fiber was able to delay the start of dissociation, but no scaffolds remained after 6 days. Incorporation of both 5% and 10% fibers in 50% norHA scaffolds were able to stop the scaffold from dissociating; however, 10% fiber incorporation retained significantly more scaffold mass after 28 days. Further, while both 5% fiber and 10% fiber incorporation were able to keep the scaffolds from dissociating, only 10% fiber scaffolds were able to maintain their shape over the course of the study, a representative image is shown in FIG. 8B. Confocal microscopy of 50% norHA scaffold with 10% fiber at day 28 showed that the internal structure of the scaffold was preserved, with porosity comparable to newly made 50% norHA scaffolds (FIG. 8C).

Further study was conducted to investigate whether fiber incorporation strengthened the mechanical properties of HMP scaffolds. Using non-destructive oscillatory rheology, the shear moduli was quantified of 100% norHA scaffolds, 75% norHA scaffolds, and 50% norHA scaffolds, with no fibers or with 5% fiber. The 5% fiber was selected for incorporation only, because it provided significant short-term reinforcement, corresponding to the time scale of this experiment. The storage moduli were measured for all groups prior and after gelatin HMP liquefication.

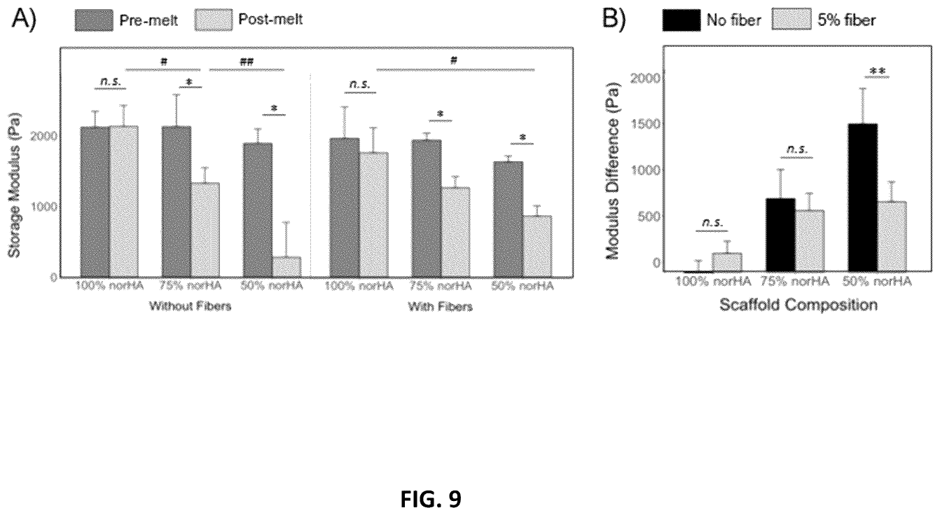

In all scaffolds with sacrificial gelatin MPs, liquefication of gelatin HMPs resulted in significant decreases in scaffold moduli (FIG. 9A−B). Prior to gelatin HMP liquefication, however, there were no significant differences in storage moduli in all groups. 5% fiber incorporation was able to rescue some of the reduction in scaffold modulus from gelatin liquefication (FIG. 9A). For 50% norHA scaffolds, the differences in the storage modulus of scaffolds prior and after gelatin liquefication were compared; statistical significance was shown between non-fiber reinforced scaffolds and 5% fiber scaffolds. For 75% norHA scaffolds, while 5% fiber incorporation reduced the decrease in storage modulus after liquefication, no statistical significance was shown. Combined, these data showed that up to 5% fiber incorporation did not reinforce the scaffolds by increasing the storage modulus of system. Rather, fiber incorporation reinforced the scaffolds by alleviating the loss in modulus resulted from liquefication of gelatin HMPs. These results suggest that low amount of fibers (5%) work to passively tether the HMPs together rather than changing the bulk properties of the scaffold. This allows for the decoupling of scaffold bulk mechanical properties and scaffold integrity, offering more control in HMP scaffold design.

### 2.4. Injection of Cell-HMP Mixture and Subsequent Crosslinking Retained High Cell Viability

Injectable biomaterials in regenerative applications allow for minimally invasive administrations of therapeutics. The self-assembling, shear-thinning, and self-healing properties of granular hydrogels make these microporous materials particularly promising for both injection and for cell culture, promoting cellular infiltration and tissue remodeling. To assess the system as an injectable, cytocompatible material, packed HMPs were extruded with cells included among the HMPs. Subsequently, the HMPs were crosslinked into a scaffold and assessed cell viability. Human umbilical vein endothelial cells (HUVECs) were used in this study to show that the granular scaffold system could support cells critical for vasculature formation.

To discern the effects of processing the cells within the material from the effects of the material itself on cell viability, viability was assessed at distinct steps during material preparation and extrusion. Specifically, a live/dead assay was performed (1) prior to the extrusion process to assess if the processing of cells within the materials was adverse to cell survival, (2) immediately after extrusion and UV cross-linking to discern the effect of injection and crosslinking on cell viability, and (3) after three days of culture to assess cell survival and proliferation in the scaffold.

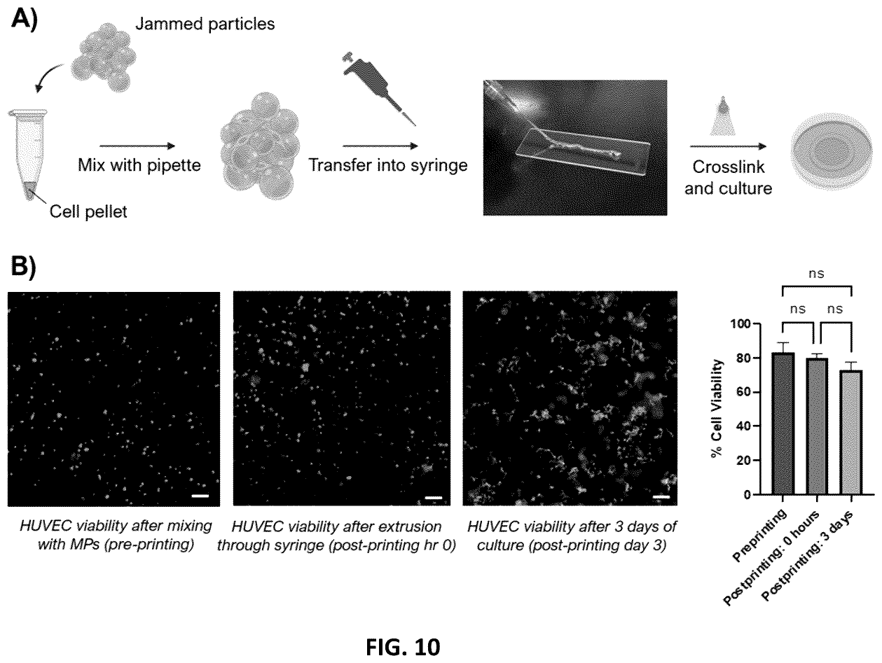

Cell viability was maintained at all steps in the process. At day 3 of culture, HUVECs were visibly adhered and spread on the HMPs (FIG. 10A−B). After the initial process of mixing cells within the packed HMPs, HUVEC viability was observed to be between 80%-90%. This, albeit being approximately 10% lower than the cell viability obtained after a normal passage of cells, preserved a degree of cell viability more than sufficient for continued culturing. This viability was maintained through extrusion, with measurement immediately after extrusion, suggesting minimal cell deaths occur from the injection and crosslinking process. After 3 days of culture, a slight reduction in cell viability was observed. This might be attributed to delayed effects of the extrusion process or to the material environment, which was minimally porous among the formulations studied. Greater permissivity is known to support cell growth. The 100% norHA scaffold is not designed to degrade or yield, and with HUVECs occupying the void space, cell growth may be stunted (FIG. 10B). Together, these results indicated that the granular hydrogel systems are biocompatible, support cell viability through extrusion, and might serve as platforms for delivering cells within biomaterials that can be designed to be highly porous. These extrudable materials might be leveraged in 3D bioprinting and regenerative engineering applications.

### 3 Experimental

3.1 Computational Custom Software analysis of 3-D pore space: norHA HMPs used for imaging was made fluorescent using AF 430 tetrazine(Lumiporbe). Briefly, norHA HMPs suspended in PBS were centrifuged at 21,000 rcf for 5 minutes. The excess PBS was discarded and 1 mM AF tetrazine was added at equal volumes to the HMPs (final concentration 0.5 mM AF tetrazine). The mixture were then vortexed to resuspend the HMPs. The reaction was allowed to proceed for 20 minutes, after which the HMP suspension were centrifuged again at 21,000 rcf for 5 minutes. The excess solution was discarded and replaced with fresh PBS. This process was repeated until the solution became clear after centrifuging the HMPs. All scaffolds were fabricated without fiber as described above. The scaffolds were imaged in PBS using a Leica confocal microscope (Stellaris). A 20× objective was used and Z-stacks of 100 μm was taken for each scaffold. Images were then processed to identify pore space and quantified using custom software.

3.2 Electrospinning norHA fibers: electrospinning solution containing 3.5% w/v norHA, 2.5% w/v 900 kDa polyethylene oxide (PEO, Sigma), 0.05% 12959 (Sigma), and 6.5 mM DTT in deionized water was dissolved overnight. The solution was extruded using a syringe pump at a flow rate of 0.4 mL/hr through a 16G needle. The fibers were collected on an aluminum foil substrate attached to a mandrel spinning at ˜1,000 rpm. 13-16 kV positive voltage was applied to the needle and 4 kV negative voltage was applied to the collection substrate.

After collection, fibers were crosslinked under UV light for 15 minutes at 10 mW/cm² under nitrogen. Once crosslinked, the fibers were wetted with PBS to detach from the collection substrate, then suspended in PBS. The suspension was homogenized at 9,000 rpm for 2 minutes and then filtered through a 40 μm cell strainer (Fisher) to eliminate large aggregations of fibers.

3.3 norHA fiber characterization: fluorescent fibers were made by incorporating 4 mg/mL FITC-dextran (1 MDa, Sigma) in the initial electrospinning solution. All other steps were the same to produce fluorescent fibers. After filtering, fibers were diluted 1:1000 in PBS, and placed between two glass cover slips. Images were taken using a Leica DMi8 widefield microscope and further dilution of the fibers was carried out until fibers no longer overlap each other in the micrograph. The images were then analyzed in FIJI to find the length and width of each fiber.

3.4 Characterizing degradation of norHA scaffolds: non-fiber scaffolds were fabricated as described above. For fiber-reinforced scaffolds, fibers were first centrifuged at 3,000 rcf to form a pellet. The pellet volume was estimated, and the pellet was resuspended 1:10 fiber to PBS volume ratio. To fabricate the scaffolds, desired volume of fiber was calculated (1%, 5%, or 10%), and corresponding volume of fiber was added to the norHA-gelatin HMP mixture (amount measured as described before for 100%, 75%, and 50%). 10 mM LAP and 25 mg/mL DTT was then added to the mixture to reach a final concentration of 1 mM for each reagent. The rest of the fabrication was carried out in the same way as described for fabricating non-fiber scaffolds.

All scaffolds for this study were fabricated on a glass coverslip substrate. Each coverslip was weighed by itself, and then weighed again with the scaffold on top to find the weight of the scaffold. Each scaffold was then submerged in PBS and cultured in a humidified incubator at 37° C. For each degradation time point, the PBS was removed by pipetting and gently wicking off the remaining PBS with a KimWipe. The weight of coverslip and scaffold was then measured again. Plotting and statistical analysis were done in Rstudio. Statistical comparison was done using ANOVA and Tukey's post-hoc test.

3.5 Rheological tests for HMP scaffolds: Time sweeps were done for HMP scaffolds to assess their mechanical properties. Scaffolds were fabricated into disks with 1 mm height and 8 mm diameter. This is done by first biopsy punching a 1 mm thick PTFE sheet (McMasters) to create an 8 mm diameter opening. The HMPs were loaded into the opening, then a glass slide is placed on top to flatten the HMPs so that they conform to the opening. Excess HMPs that overflowed between the glass slide and the PTFE sheet were simply wiped away after the glass slide is removed. The HMPs were then photo-crosslinked for 1 minute under 10 mW/cm² UV light.

Rheology was conducted on a Peltier plate temperature controlled at 20° C. The fabricated 8 mm scaffolds were loaded onto the Peltier plate and positioned under an 8 mm sandblasted parallel plate geometry. Each scaffold is conditioned by applying a small axial force (0.3-0.8 N) prior to oscillatory testing. This accounts for the potential uneven topography produced during scaffold fabrication and ensures that all scaffold is sufficiently contacting the geometry. Time sweeps were done at 1% strain and 1 Hz frequency for 60 seconds.

To assess mechanical properties of scaffolds post gelatin HMP sacrifice, after time sweep was done for the pre-sacrifice scaffolds, the scaffolds were submerged in PBS and cultured for 20 minutes at 37° C. After culturing, the scaffolds were gently washed with excess PBS, also at 37° C., to remove liquefied gelatin. The same time sweep with conditioning was conducted again on the scaffolds. Statistical analysis was done using paired t-tests for these samples.

3.6 Injection of HUVEC and HMPs: HUVECs (Lonza) was cultured in EGM-2 medium (Lonza) at 5% CO2 and 37° C. in a humidified environment. HUVECs Passage 6-8 was used for this experiment. HUVECs were detached from plate using 0.05% trypsin, centrifuged at 300 rcf for 3 minutes, and resuspended in EGM-2 at 30 million cells per mL.

RGD-modified norHA HMPs were made by mixing the norHA HMPs with RGD and LAP to reach a final concentration of 1 mM RGD and 1 mM LAP (norHA HMPs comprises half the volume of the solution). This mixture was placed under UV light at 10 mW/cm² for 5 minutes, then washed twice with PBS to eliminate excess RGD and LAP.

The RGD-norHA HMPs were centrifuged at 21,000 rcf for 5 minutes. The dense HUVEC suspension was then mixed with the HMPs to reach a final density of 2 million cells/mL. The mixture was then loaded into a 1 mL syringe and subsequently extruded onto a 6-well cell culture plate through a bevel, 18G needle. The material was subsequently UV crosslinked at 15 mW/cm² for 1 minute.

For live/dead staining, HUVECs-HMPs mixture were collected prior to loading the material into the syringe, while post-printing mixtures were collected either immediately after extrusion or after 3 days of culture. The cells were stained using a Live/Dead viability kit (L3224, Invitrogen) and imaged in a plastic-bottom culture plate by a Leica DMi8 widefield microscope. Statistical analysis was performed using GraphPad Prism 9. Statistical comparison was made using one-way analysis or variance (ANOVA) with Tukey post hoc test.

### 4 Conclusions

NorHA was synthesized by conjugating a norbornene group to the hydroxyl group of HA. From here, norHA HMPs were fabricated using batch emulsification. norHA HMPs were fabricated with varying sizes and moduli rapidly using this method. Through efficient norbornene-thiol click chemistry, norHA HMPs was secondarily crosslinked to each other, as well as conjugated with thiolated RGD to facilitate cell growth. Introducing a population of sacrificial particles was shown to tune the porosity and create multi-scale, interconnected pores. Porous scaffold with as much as 50% porosity can be made viable for long term culture through the introduction of electrospun fibers. 10% v/v fiber incorporation was able to stabilize scaffolds with 50% porosity over a period of 30 days, retaining both the scaffold porosity and structure. Finally, the generated HMPs were shown to be suitable for cell culture; and that HUVECs were able to survive mixing, injection, as well as subsequent UV crosslinking in the HMPs with high viability. In conclusion, this work presents an approach to engineering high degrees of porosity in granular hydrogel systems that is high-throughput and well-characterized. Additionally, reinforcement of crosslinked HMPs enabled by small amounts of electrospun fibers have not been previously explored; and is shown here to be a viable method to reinforcing even highly porous HMP systems. Combined, the methods presented in this paper may enable an additional degree of freedom in designing granular materials for various tissue engineering and regenerative medicine applications.

### Example 2

Viscoelasticity and stress relaxation are critical ECM properties that are known to influence cell behaviors, thereby driving the recent emphasis on incorporating these complex mechanics into 3D hydrogel culture models. Granular hydrogels are an emerging class of 3D scaffolds due to their inherent viscoelasticity, as well as their micro-to-mesoscale void space within the scaffold, which provides cells with degrees of migratory freedom that are not possible in conventional 3D bulk hydrogels. Traditional hydrogel microparticles (HMPs) are often spherical (aspect ratio ˜1) and thus restricted to only contacting immediately adjacent HMPs. This limited range of interaction between particles complicates the ability to tune time-dependent mechanics like stress relaxation because particles easily shift past each other in response to strain. In response, a system that leverages segmented hydrogel microfibers (aspect ratio ˜15) as the individual “grains” within the granular hydrogel is described herein, which enables these interactions at increased length scales compared to their spherical analogs. The fiber-based granular hydrogel system exhibits viscoelasticity, shear-thinning and self-healing properties, and injectability—like all other classes of granular hydrogels. This increased length scale of interaction allows for fibers to entangle and slide past each other, which enables tunable stress relaxation profiles (T_{1/2}˜1-100+s) across a range of applied strains (σ˜2.5-50%) in a packing density-dependent fashion—behaviors that were not seen with spherical particles with matched volume and matched dimension. The ability to selectively anneal a small subset of fibers within the granular hydrogel to bolster scaffold mechanics. Taken together, fiber-based granular hydrogels offer an intriguing alternative to traditional granular hydrogels due to their unique mechanics, and offer a promising solution to engineering complex 3D scaffolds for cell culture applications.

### 1 Introduction

Mechanotransduction of extracellular matrix (ECM) mechanics is widely considered to be a critical driver of many fundamental cell behaviors, such as proliferation, migration, and differentiation. The components of the ECM are fairly well characterized, along with their contribution to overall mechanical properties, thereby providing a blueprint of desired characteristics when engineering in vitro models of various tissue systems. The most well-described and studied ECM mechanical property is its stiffness or elasticity; however, natural tissue is not purely elastic, with viscous contributions (i.e., viscoelasticity) that are increasingly complicated by strain- and stress-dependent behaviors. These characteristics are exploited by cells within the environment during routine processes. More specifically, cells within the ECM exert protrusion and traction forces during migration where they reorganize the ECM and contribute to localized nonlinear stiffening of the matrix. Therefore, when developing an in vitro biomaterial system to model natural tissue, the resultant scaffold must be compliant within physiologically-relevant force and strain regimes to provide cells an environment that recapitulates endogenous complex matrix mechanics.

Hydrogel-forming biomaterials are widely leveraged to engineer ECM-mimetics due to their ability to match many of the biophysical and biochemical attributes of natural tissue. While there are numerous proven strategies to directly control hydrogel stiffness, these are commonly implemented as 2D tissue culture substrates. Indeed, much of the established knowledge surrounding fundamental cellular functions is predicated on 2D studies utilizing static hydrogel microenvironments. Due to the increased appreciation for the complexity of ECM mechanics in 3D, there has been a shift to developing hydrogels that enable more dynamic microenvironments for use as 3D tissue models. Naturally-derived materials like collagen and fibrin offer a simple platform for introducing viscoelasticity and stress relaxation into hydrogels, but their low overall modulus frustrates their utility as tunable biomaterials systems. These challenges have inspired a shift towards developing materials systems with advanced chemical crosslinking strategies that enable more complex mechanical profiles. Engineering controlled viscoelasticity and stress relaxation into hydrogels is perhaps the most common strategy for increasing the dynamicity of 3D culture environments, and prevalent examples include dynamic guest-host chemistries grafted onto various polymer backbones and physical (ionic) chelation of polymers like alginate. When coupled with covalent crosslinking mechanisms, these reversible interactions allow for increased control over various ECM-mimetic mechanics, providing useful platforms for 3D cell culture. Importantly, while these strategies enable a tunable approach to engineering viscoelasticity into 3D hydrogel environments, they often spatially confine cells through the crosslinked nanoscale hydrogel network.

Granular hydrogels are a class of 3D hydrogel environments that address the spatial restrictions that cells experience in bulk hydrogels due to their inherent void space in the scaffold, and there has been considerable work utilizing these materials for 3D cell culture. These hydrogel systems are comprised of discrete hydrogel microparticles (HMPs) that are packed together and immobilized strictly by contact forces between discrete particles. The micro-to-mesoscale space between the particles enables cells to migrate throughout the overall construct in ways that are challenging in bulk 3D hydrogels. While granular hydrogel scaffolds provide a more facilitative environment for migratory processes, the ability to tune viscoelasticity and stress relaxation is largely governed by the minimal length scale of interactions (i.e., contact forces between particles) between individual HMPs compared to the crosslinked polymer chains in bulk hydrogels. Enabling longer-range interactions within granular hydrogels is well-demonstrated via annealing particles together to enable force transmission across increased length scales; however, this often limits the permissive nature of the scaffold by covalently immobilizing particles in place.

In response to these challenges, a novel granular hydrogel system that enables the desired long-range interactions with tunable viscoelasticity and time-dependent stress relaxation was developed, without requiring secondary annealing. Inspired by the fibrous proteins that contribute to complex matrix mechanics in endogenous tissue, It is hypothesized that a granular hydrogel comprised of high-aspect ratio, flexible fiber segments as the “grains” might afford interactions at longer length-scales due to fibers siding and entangling—contributing to scaffold mechanics similar to the proteins in native ECM. Herein, a fiber-based granular hydrogel system that exhibits packing density-mediated viscoelasticity and stress relaxation in response to physiologically-relevant forces and strains is disclosed. These behaviors are unique to this new class of granular hydrogels, where spherical particles with matched volume and matched dimension (i.e., particle diameter—fiber length) are unable to exhibit similar properties under the same conditions designed for this study. Fiber-based granular hydrogels are soft and tunable, suggesting their utility as 3D in vitro models of soft tissue types, with the possibility of selective covalent annealing to increase mechanics and model systems with greater stiffnesses.

### 2 Materials and Methods

### 2.1 Peptide Synthesis

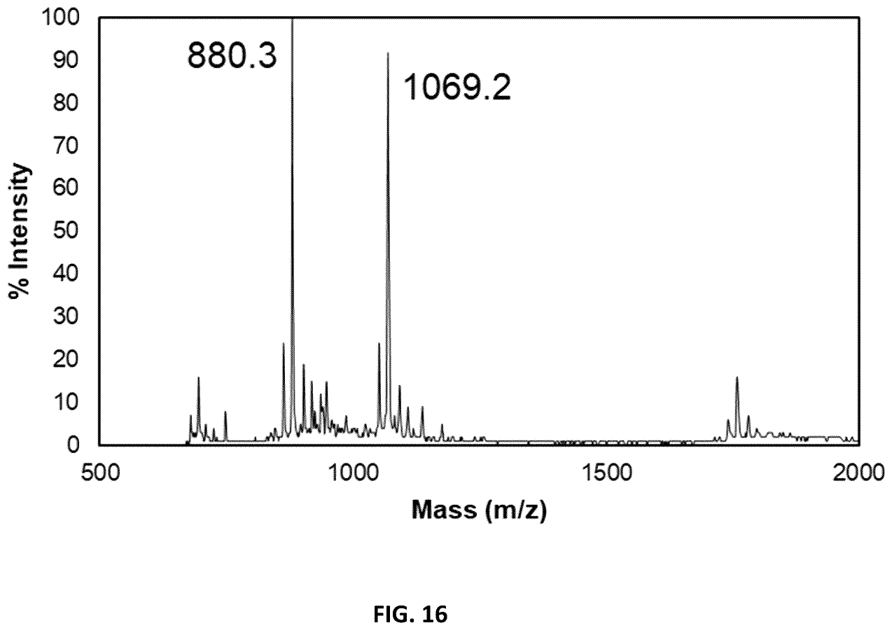

The fluorescent peptide (GCDDD-fluorophore) utilized to visualize microfibers and microgels in this study was synthesized with a cysteine residue to permit thiol-ene conjugation to residual norbornenes during the fiber- and particle-making processes (Liberty Blue automated, microwave-assisted solid phase peptide synthesizer, CEM). The peptide was built from C-terminus to N-terminus on Rink amide resin using Fmoc-protected amino acids (resin and amino acids were sourced from Advanced Chemtech), with 5(6)-carboxyfluorescein (FAM, Sigma Aldrich) added last to the N-terminus. The resultant peptide was cleaved off the resin using a cocktail of trifluoroacetic acid, triisopropylsilane, 2.2′-(ethylenedioxy) diethanethiol (all were sourced from Sigma Aldrich), and DI water at a 92.5/2.5/2.5/2.5 mixing ratio, respectively. The freed peptide was then isolated via precipitation in cold diethyl ether (Sigma Aldrich), dried under vacuum, resuspended in DI water, and lyophilized to yield the final product. Peptide synthesis was confirmed using MALDI-TOF spectrometry (FIG. 16).

### 2.2 Electrospinning and Segmenting PEGNB Microfibers

To electrospin PEGNB, solutions comprised of 10% w/v PEGNB (20 kDa, JenKem Technology), 7% w/v PEGSH (10 kDa, JenKem Technology), 5% polyethylene oxide (PEO, 400 kDa, Sigma Aldrich), and 0.05% w/v 2-hydroxy-4′-(2-hydroxyethoxy)-2-methylpropiophenone (HHMP, Sigma Aldrich) in DI water were mixed overnight. Quantity of PEGSH was determined to enable a stoichiometric mismatch between norbornene and thiol groups to avail residual norbornenes for coupling with thiolated peptides. 0.5 mM of thiolated FAM peptide (GCDDD-FAM) was included in the electrospinning precursor solution to enable fluorescent imaging of microfibers for visualization and characterization. A second electrospinning solution designed to be co-spun with the PEGNB solution comprised of 5% PEO (900 kDa, Sigma Aldrich) in DI water was also mixed overnight. This solution was intended to yield sacrificial fibers that would introduce space between PEGNB fibers during the electrospinning process, but dissolve away after the crosslinking step to prevent welding of PEGNB fibers.

The PEGNB and sacrificial fiber solutions were both extruded through 16-gauge needles positioned 18 cm away from a rotating mandrel collector at rates of 0.4 ml/hr and 0.5 ml/hr, respectively. The needles were charged with 11-12 kV for the PEGNB solution and 5.5-6.5 kV for the sacrificial fiber solution. The mandrel was charged with −4 kV to focus the electrical field and set to rotate at 1000 RPM to align fibers and minimize welding. Fiber batches were collected for 1 hr each, crosslinked under nitrogen for 15 min at 5 mW/cm² (VWR UV Crosslinker) to stabilize the PEGNB fibers, then hydrated in PBS to hydrate PEGNB fibers while simultaneously dissolving away the sacrificial fibers. Fiber batches were hydrated overnight to ensure sufficient swelling of the hydrogel network and removal of undesired components from the electrospinning process (i.e., PEO and unreacted photoinitiator).

Hydrated fiber mats were suspended in PBS at ˜10% v/v, segmented via homogenization (IKA T25) at 10 k RPM for 2 min, and filtered through a 40 μm mesh to remove welded fiber aggregates. Final solutions were centrifuged thrice to remove all undesired components, and fibers were resuspended at 10% v/v and stored at 4° C. until use. Fiber length and diameter were characterized using ridge detection in ImageJ based on thresholded confocal images of dilute fiber solutions (BioTek Cytation C10, Agilent Technologies).

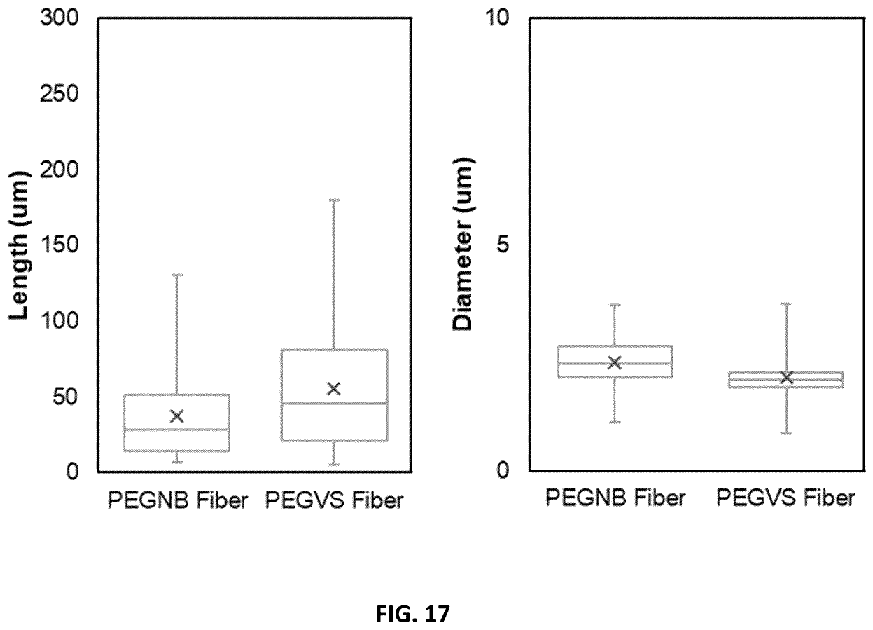

For crosslinkable PEGVS fibers, the electrospinning solution was prepared similarly to the PEGNB solution, with 10% w/v PEGVS (10 kDa, Jenkem Technology) replacing both the PEGNB and PEGSH since PEGVS crosslinks readily with itself without a crosslinker molecule. PEGVS was electrospun utilizing the same parameters as PEGNB, crosslinked for 5 min at 5 mW/cm², then subsequently processed using the same protocol established for the PEGNB fibers. PEGVS microfiber characterization is included in FIG. 17.

### 2.3 Aqueous Two-Phase PEGNB Hydrogel Microparticle Synthesis

To form PEGNB hydrogel microparticles via aqueous two-phase suspension, solutions of dextran from Leuconostoc spp. (70 kDa, dextran(70), Sigma Aldrich) were mixed with PEGNB hydrogel precursor solutions at a 4:1 ratio of continuous(dextran):disperse(PEGNB) phases. For spheres (V), 800 μl of 40% w/v dextran and 0.05% HHMP in DI water was mixed with 200 μl of a solution comprised of 6% PEGNB, 4.2% PEGSH, and 0.05% HHMP in DI water. 0.5 mM of GCDDD-FAM peptide was included in both phases as well when preparing particles for fluorescence imaging. This mixture was vortexed at maximum speed for 1 min prior to crosslinking under UV light at 20 mW/cm² (Omnicure) for 5 min. For spheres (D), the continuous dextran phase was modified to 25% w/v and the stir rate was reduced to 800 RPM, but all other parameters were conserved compared to spheres (V). Following the crosslinking step, the resultant particles were suspended in 15× volume of PBS to thermodynamically favor a single phase solution and centrifuged twice to remove dextran and other unreacted materials. Spheres (V) and spheres (D) were filtered through 20 μm and 40 μm meshes, respectively, and centrifuged once more to yield the final particles used within this study. Particles were also stored at 10% v/v in 4° C. until further use. Similar to fibers, particle size was characterized using ImageJ based on thresholded confocal images of dilute particle solutions (BioTek Cytation C10, Agilent Technologies).

### 2.4 Forming Granular Hydrogels

Two classes of granular hydrogels were designed for this study: those comprised of segmented PEGNB fibers and those comprised of spherical PEGNB microparticles. Fibrous granular hydrogels were assembled via centrifugation at 5 k, 10 k, and 15 k RCF for 5 min to yield low, medium, and high packing densities, respectively. Spheres (V) and spheres (D) were packed at the medium packing density to enable direct comparisons with the fibrous assemblies packed at medium density. Following centrifugation, the supernatant was carefully aspirated to avoid disrupting the granular hydrogel and the pellet was manipulated using a spatula thereafter.

### 2.5 Granular Hydrogel Characterization

To characterize void space in these granular hydrogel assemblies, fibers and spheres were resuspended in 2 mg/ml FITC-dextran (2 MDa) then subsequently centrifuged to yield the desired packing density. In this scheme, the fluorescent regions represent the void space within the assembly. Packed granular hydrogels were then transferred to a 96 well plate and Z-stacks of each sample were acquired at random ROIs on a Leica Stellaris 8 confocal microscope. Images were thresholded on ImageJ and void space was quantified using the built-in Analyze Particles functionality. Void space was determined as the average pixel intensity of the fluorescent regions with respect to the total pixel volume of the micrograph for each group.

Mechanical properties of granular hydrogel assemblies were assessed via oscillatory shear rheology (DHR-3, TA Instruments) using a 20 mm parallel plate geometry, a 500 μm gap distance, and a 25° C. testing temperature. Time sweeps (0.5% strain, 1 Hz) were utilized to assess viscoelasticity of granular hydrogels. Cyclical addition of high and low strains (low: 0.5%, 1 Hz; high: 250%, 1 Hz) were leveraged to demonstrate shear recovery. Strain sweeps (0.01%-500% strain) helped elucidate strain yielding and critical strain values. Finally, constant application of strain (ranging from 2.5%-50%, depending on the trial) was utilized to investigate stress relaxation characteristics of granular hydrogel assemblies.

For annealed particle systems using varied quantities of PEGVS, the Peltier plate base was replaced with a glass base that enables UV curing. Fibrous assemblies were resuspended in a 0.05% w/v HHMP solution, centrifuged to yield the medium packing density, then transferred to the rheometer. Time sweeps (0.5%, 1 Hz) with UV curing at 5 mW/cm² (either 10 s or 120 s, Omnicure) were utilized to assess the crosslinked scaffold mechanics, followed by a 15% constant strain to record stress relaxation capabilities of partially-annealed scaffolds. Noisy stress relaxation data were smoothed using an exponential smoothing algorithm.

### 3 Results and Discussion

### 3.1. Preparing Granular Hydrogels

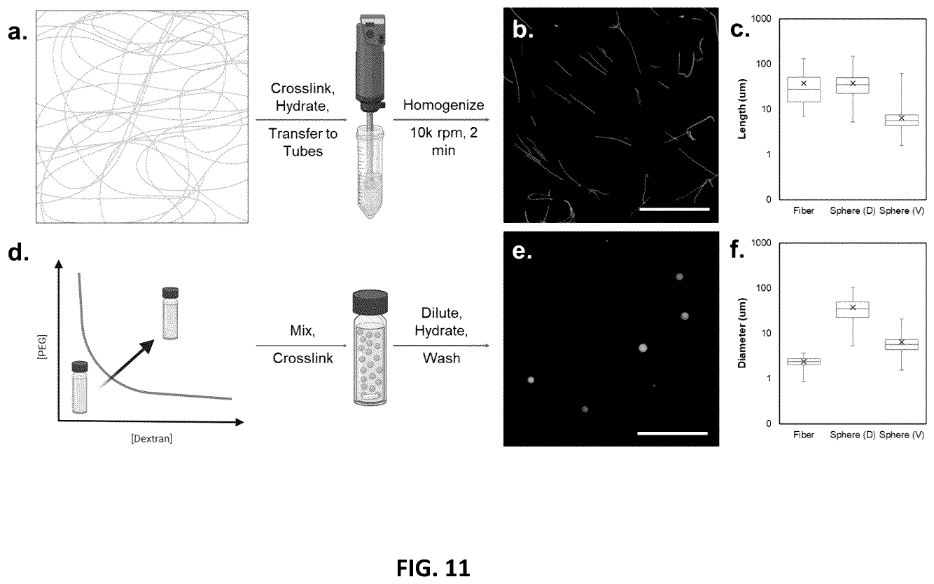

The two types of granular hydrogels utilized in this study were both prepared using modified polyethylene glycol (PEG) backbone chains. PEG is a hydrophilic polyether that is readily modifiable with functional groups for various crosslinking mechanisms. Furthermore, PEG is relatively bioinert—lending itself as a “blank-slate” material that offers user-defined tuning of the biochemical profile through the installed reactive groups. Here, PEG-norbornene (PEGNB) and PEG-thiol (PEGSH) were selected as the modified PEG derivatives due to the stoichiometric nature of the photomediated step-growth thiol-ene click reaction mechanism. This chemical scheme allows for residual norbornene groups following the crosslinking process to enable additional thiol-ene conjugation of thiol-containing fluorophores and bioactive peptides. For granular hydrogels with high aspect ratios to investigate long-range entanglements, segmented electrospun microfibers were utilized as the individual grains (FIG. 11A−B). As comparisons, spherical hydrogel microparticles were prepared via an aqueous two-phase separation technique (ATPS, FIG. 11D-E) to yield particles with matched dimension (i.e., particle diameter—fiber length) and matched volume (assuming spherical for particles and cylindrical for fibers)—hereafter “spheres (D)” and “spheres (V)”, respectively (FIGS. 11C and 1F).

Electrospinning is an effective platform for fabricating hydrogel fibers with diameters on the order of hundreds of nanometers to single microns. Here, the electrospinning setup was modified such that the resultant hydrated fibers were >1 μm in diameter (average diameter=2.41 μm, FIG. 11F). This design criterion was implemented under the assumption that microscale diameters would enable increased porosity over nanoscale diameters. Previous methods to segment electrospun hydrogel fibers include repeated aspiration and extrusion steps, photopatterning, and cryomilling; however, a simple and scalable homogenization step was employed where fiber solutions were agitated at 10 k RPM for 2 min, then filtered to remove aggregates. Following the processing steps, the resultant fibers were measured to be approximately 37.3 μm in length (FIG. 11C). The aspect ratio (L/D) of ˜15 is considerably larger than most hydrogel microparticles used for granular hydrogel scaffolds.

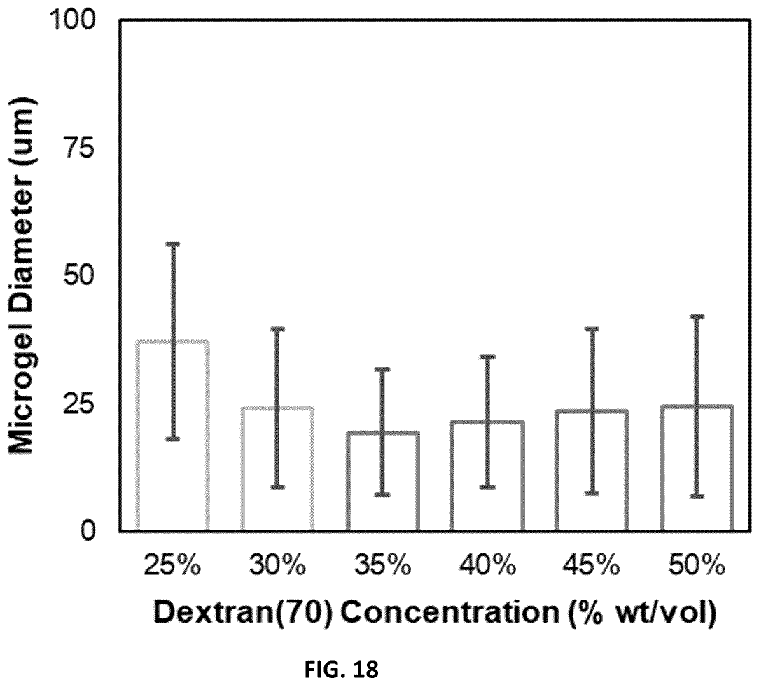

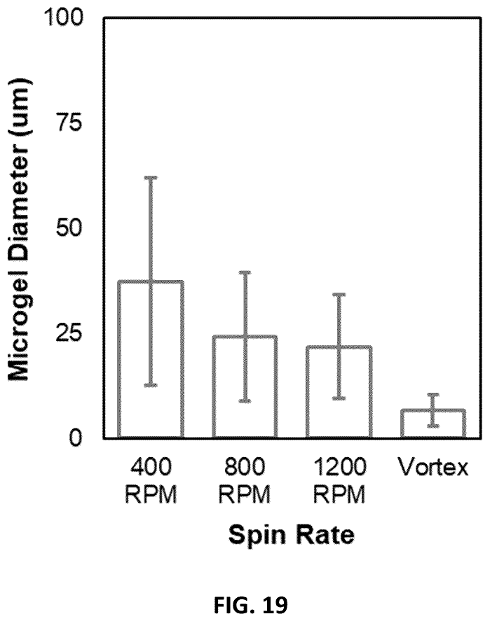

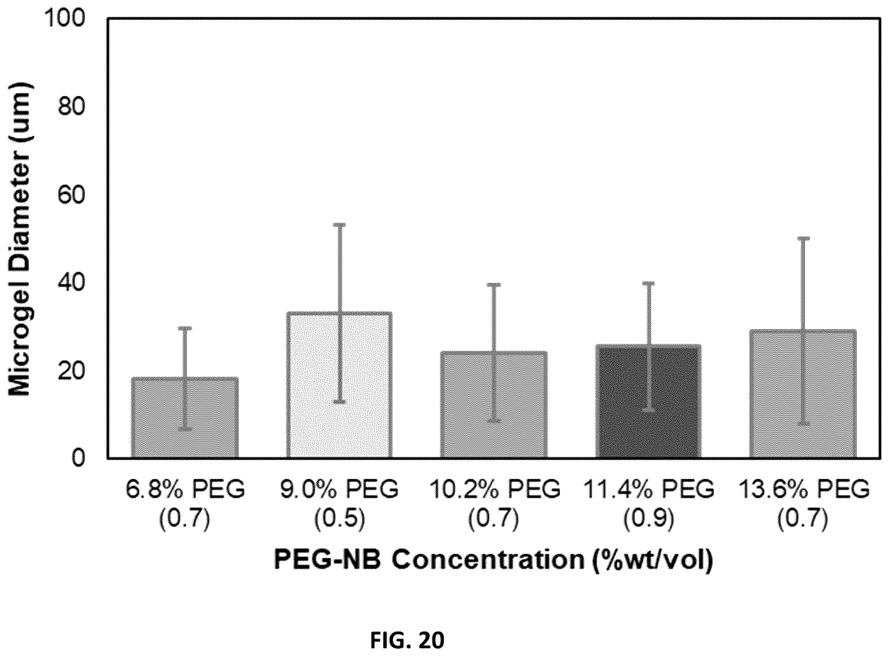

In order to compare fiber-based granular hydrogels/PHMs to more traditional hydrogel microparticles, PEGNB spheres (D) and spheres (V) were produced using an ATPS technique. This process leverages PEG-rich and dextran-rich aqueous solutions that are thermodynamically immiscible when mixed at high enough concentrations. A systematic approach to determine experimental concentrations of PEGNB/PEGSH and dextran solutions was leveraged to form PEGNB hydrogel microparticles (summary of different processing variables shown in FIG. 18-20). Spheres (D) have an average diameter 37.2 μm which matches the average length of the electrospun fibers, and spheres (V) have an average diameter of 6.4 μm which approximately matches the volume of the electrospun fibers. Matching these dimensional characteristics of the electrospun fibers allows for investigating how the increased aspect ratio of PEGNB fibers affects mechanical properties of resultant granular hydrogels when compared to spherical hydrogel microparticles.

### 3.2 Influence of Particle Size and Shape on Granular Hydrogel Properties

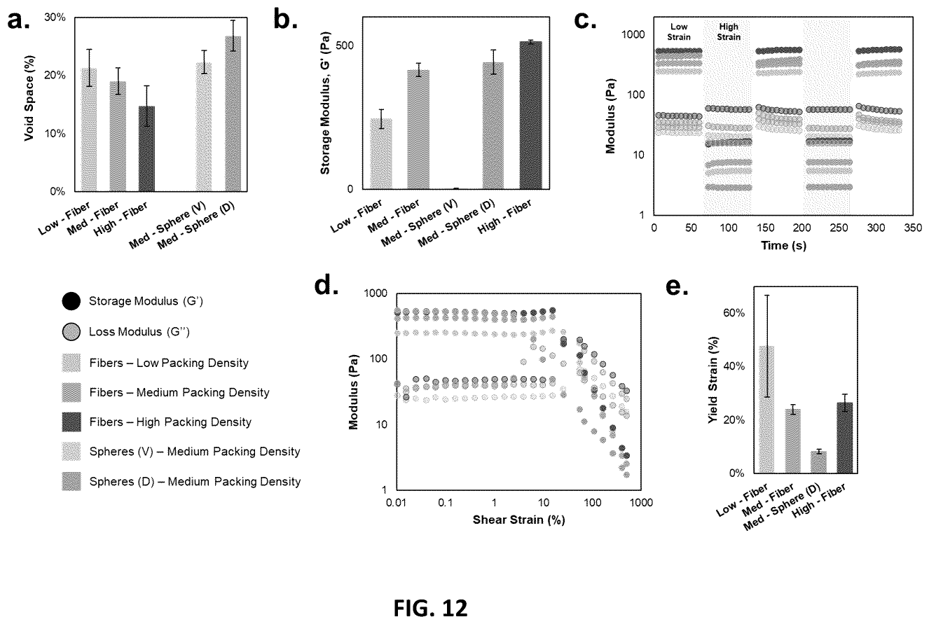

Granular hydrogels were formed via centrifugation-mediating packing at different speeds to yield low, medium, and high packing densities—hereafter “Low-Fiber”, “Med-Fiber”, and “High-Fiber”, respectively. Fibers were packed at all three densities whereas spheres (V) and spheres (D) were only packed at the medium density for comparison to fibers (“Med-Sphere (V)” and “Med-Sphere (D)”). To investigate differences in porosity of the granular hydrogels, particles were packed with high-molecular weight FITC-dextran and confocal microscopy was leveraged to visualize the fluorescent signal within pores (FIG. 12A). Consistent with previous findings in other types of granular hydrogel systems, increasing packing density of fibers resulted in decreased void space (from 21% for Low-Fiber to 15% for High-Fiber). Both Med-Sphere (V) and Med-Sphere (D) possessed larger quantities of void space in the granular hydrogel (22% and 27%, respectively) when compared to the fibers. This is likely due to the small diameters and flexibility of the fibers allowing for fibers to fold and fill more void space following centrifuge-mediated packing when compared to spheres that maintain their defined shape following packing.

It is evident that all packing densities for fibers behave like solids at rest as illustrated by their storage modulus ranging from ˜245-513 Pa (FIG. 12B). Similarly, Med-Sphere (D) exhibit storage moduli values of ˜440 Pa, which is consistent with the moduli seen with the Med-Fiber group. Conversely, Med-Sphere (V) did not exhibit an appreciable storage modulus (<5 Pa). These results suggest that at the packing densities investigated, the characteristic dimension of the fibers and spheres (D) is the main contributor to the storage moduli at low strains, with the smaller diameters of spheres (V) unable to generate sufficient interparticle contact forces to sustain solid-like behavior. Thus, for the rest of the rheological analyses, only spheres (D) were analyzed in parallel with fibers.

A favorable characteristic of granular hydrogels is their ability to be manipulated via pipetting or injecting, which is advantageous for creating permissive 3D scaffolds for in vitro cell culture. When cycling between low strain (0.5%) and high strain (250%) regimes, all granular hydrogels demonstrated shear-thinning and self-healing properties and were able to restore a majority of their original mechanics, regardless of particle shape (FIG. 12C). Additionally, all groups of granular hydrogels are shear-yielding (FIG. 12D-E), with yield strains (% strain where G′<G″) affected in both a packing density- and particle shape-dependent manner. More specifically, the yield strain (˜48%) for the Low-Fiber group trended higher than the yield strains for the Med-Fiber and High-Fiber groups (24% and 26%, respectively). This phenomenon may be attributed to the higher interstitial fluid content in the Low-Fiber group effectively providing space for the fibers as they slide and reorganize within the granular hydrogel environment in response to the increasing applied strain. At higher packing densities, fibers are still sliding and reorganizing, but their mobility might be more limited as the fibers are packed closer together, thereby minimizing their ability to respond accordingly to the applied strains. Conversely, Med-Sphere (D) exhibited yield strains considerably lower than all fiber groups (˜8%). It is hypothesized that this difference is due to the inability of spherical microparticles to interact with more particles besides those immediately adjacent, thus limiting longer-range interactions. Alternatively, fibers are able to entangle and interact with many other fibers at longer ranges since their aspect ratios are much larger than spheres, even if the characteristic length of both particle types are consistent (fiber length≈sphere diameter).

The long-range entanglements in fiber-based granular hydrogels might be useful for developing permissive scaffolds for 3D cell culture. Previously, long-range interactions between particles were generally only afforded when secondary annealing was introduced to enable force- and stress-transmission across increased length scales. The entanglements between fibers allow for enhanced flexibility when engineering scaffolds that mimic complex matrix mechanics. More specifically, cells within the ECM are known to exert protrusion and traction forces during migration on the order of 10⁻¹-10¹ kPa, coupled with 10-50% strains where they reorganize the ECM. Within the design parameters of this study, fiber-based granular hydrogels are able to withstand strains within this range without yielding, unlike spheres with matched dimension and spheres with matched volume—therefore potentially providing a platform that enables cells to behave like they would in vivo.

### 3.3 Viscoelasticity of Granular Hydrogels

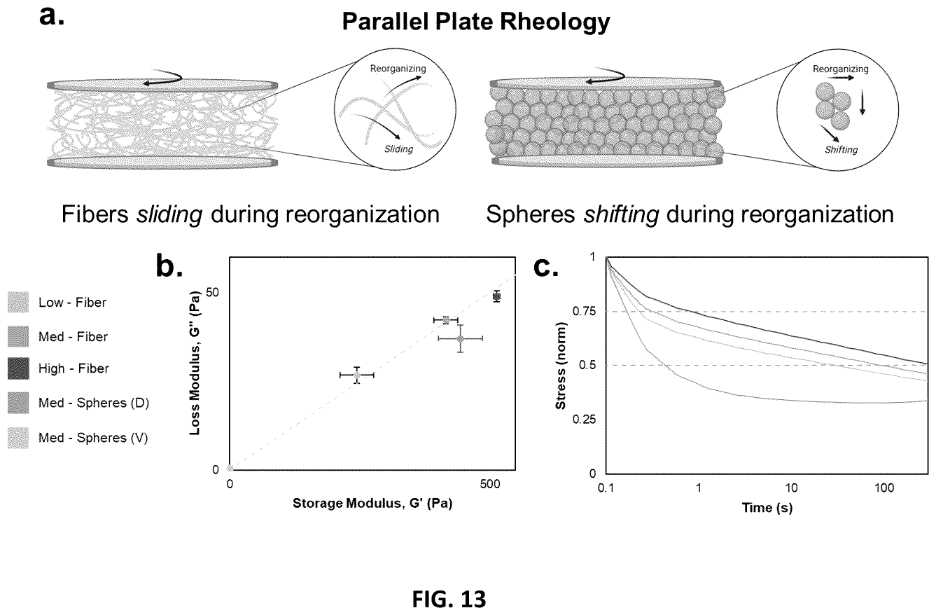

Viscoelasticity is an increasingly appreciated characteristic of natural tissue that is implicated in many cell processes. All tissues exhibit varying levels of viscous behavior which facilitates the tissue's ability to dissipate stress in a time-dependent manner when responding to applied forces. This understanding has motivated the need to incorporate viscoelasticity with tunable stress relaxation profiles into engineering biomaterials scaffolds. Therefore, the elastic and viscous contributions to the overall mechanics of the granular hydrogels described here were quantified using oscillatory shear rheology. It is hypothesized that the long-range entanglements afforded by the electrospun fibers would allow for increased viscoelasticity over their spherical counterparts—due to the ability of the fibers to slide and reorganize over greater length scales compared to spheres shifting in response to applied strains (schematic in FIG. 13a).

Elastic and viscous contributions to the mechanical properties of natural tissue and ECM-mimetics are often visualized by plotting their storage modulus versus their loss modulus (FIG. 13b). Many tissues exhibit an elastic contribution that is ˜10× the viscous contribution (shown as the gray dashed line in FIG. 13b), so that is the target criterion when designing these granular hydrogels. Indeed, fiber-based granular hydrogels all demonstrate storage moduli that are ˜10× the loss moduli, therefore achieving that desired design criterion. Med-Spheres (D) have an increased elastic contribution as illustrated by their deviation from the 10× trendline in FIG. 13b, suggesting that the sliding and reorganizing abilities of fiber-based granular hydrogels might be advantageous for viscoelasticity. This phenomenon is likely analogous to reversible chemistries that are known to increase viscoelasticity of bulk hydrogels. For example, a hydrogel system crosslinked via a supramolecular guest-host mechanism (i.g., adamantane-β-cyclodextrin) would exhibit increased viscous behavior as applied forces disrupt that guest-host interaction, but this is reversible if the force is removed before the polymeric network has undergone substantial rearrangement to relax the applied stress. It is postulated that fiber-based granular hydrogels exhibit similar response—except at the microscale as opposed to the nanoscale—where fibers begin to slide in response to applied forces but return to rest due to frictional and entanglement forces when the force is removed.

Time-dependent stress relaxation is thus another important ECM mechanical property that is closely related to viscoelasticity. Natural tissue dissipates stress at relaxation times (T_{1/2}, defined as the time it takes for a tissue or material to relax to 50% of the peak stress under constant strain) ranging from 1-1000 s. Therefore, it is desirable to develop a tunable materials system that is able to respond to constant applied stresses similarly. Using a constant applied shear strain of 15%, all packing densities of fiber-based granular hydrogels were found to be able to dissipate stress with T_{1/2}>10 s (FIG. 13c). Additionally, T_{1/2} demonstrates a positive correlation with packing density, where the relaxation time is longest for the High-Fiber group. In comparison, Med-Spheres (D) exhibited a sharp drop off in normalized stress with a T_{1/2}<1 s. This is likely due to the limited range of interactions between spheres preventing them from sliding against each other and instead shifting past each other. Moreover, 15% strain is higher than the yield strain for Med-Spheres (D) (˜8%, FIG. 12e), suggesting that spherical hydrogel microparticles are unable to dissipate stress over lengthened time scales if the applied strain surpasses the yield strain.

### 3.4 Strain-Dependence of Granular Hydrogel Stress Relaxation

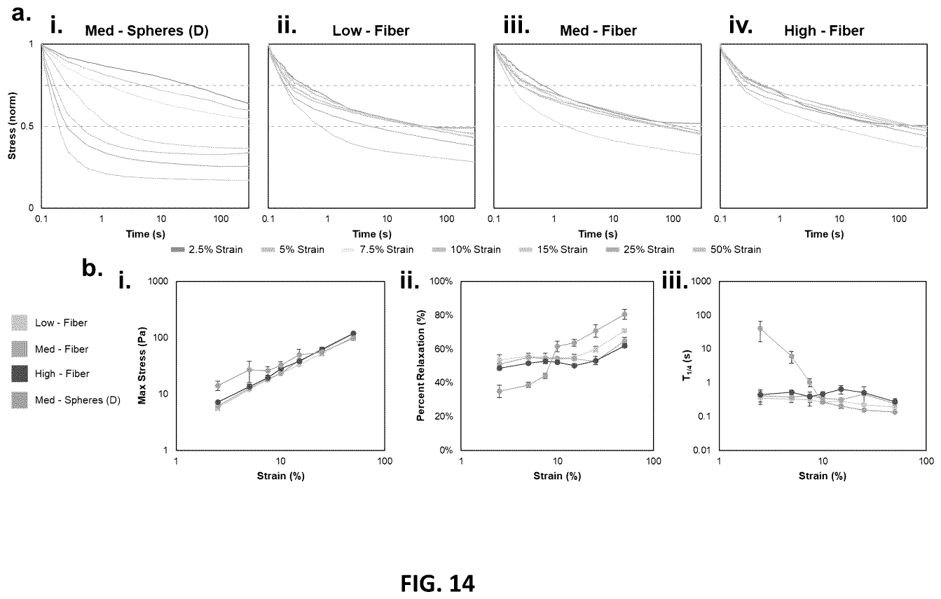

Preliminary analysis of time-dependent stress relaxation of granular hydrogels in this study demonstrated that Med-Spheres (D) relax at time scales on the order of 10⁻¹ s, which is likely related to their yield strain. Med-Spheres (D) responded to a range of applied strains (2.5-50% strain), with a particular focus on strains that do not supersede their yield strain (FIG. 14ai). The stress-dissipation behaviors of fiber-based granular hydrogels within this same range were compared, aiming to identify a relationship between yield strain and relaxation time similar to Med-Spheres (D) (FIG. 14aii-iv). Importantly, a different definition of relaxation time is used here, T_{1/4}, which is when the material system only relaxes 25% of the max stress value. This different relaxation time allows for us to draw comparisons across groups that do not exhibit a T_{1/2} value within the 300 s test parameter utilized here.

Illustrated in FIG. 14ai, it is evident that there is a distinct strain-dependent relationship for the stress relaxation profiles for Med-Spheres (D). As previously demonstrated, a yield strain of ˜8% for the Med-Spheres (D) granular hydrogel was observed. For applied strains <8%, Med-Spheres (D) are able to slowly relax as spheres slide in place to dissipate stress. However, once the applied strain reaches 10% and above, Med-Spheres (D) exhibit a sharp decrease in normalized stress as sphere-sphere surface contact is effectively disrupted in response to applied strains greater than their yield strain. Critically, the range of strains in which Med-Spheres (D) relax in T_{1/4} values greater than 1 s is outside the range of strains considered relevant for most cell activity (10-50%), suggesting that further modifications might be necessary to engineer ECM-mimetic environments using the Med-Spheres (D) scaffolds described here.

In comparison, all packing densities of fiber-based granular hydrogels exhibit a muted relationship between applied strain and stress relaxation (FIG. 14aii-iv). This observation is particularly noticeable for strains ranging from 2.5-15% strain for all packing densities, with deviations beginning at 25% strain for the Low-Fiber group and 50% strain for the Med-Fiber and High-Fiber groups. Again, these phenomena are attributed to the high aspect ratio of the fibers entangling within the granular hydrogel at increased length scales, enabling sliding and reorganizing in response to applied stress. Additionally, while there is a slight change in behavior of T_{1/4} around the yield strains of fibers, no noticeable decrease in normalized stress is observed when compared to Med-Spheres (D). This result could be explained by the ability of fibers to reorganize under a constant applied strain near their yield strain in a manner in which spheres are unable to replicate.

Further study was carried out to quantify the relationships observed in the stress relaxation profiles to support the comparisons drawn between Med-Spheres (D) and all packing densities of fiber-based granular hydrogels. Interestingly, when comparing the max stress value for all groups, they exhibit the same linear trend where max stress increases with applied strain (FIG. 14bi). It is noteworthy that Med-Spheres (D) exhibit a higher max stress than fibers at low strains, but this trend begins to diminish once applied strains surpass the yield strain of the spheres. When analyzing the percent relaxation (FIG. 14bii), similar trends are observed across all fiber groups where total relaxation increases modestly at low applied strains, but sharply increases once the applied strain surpasses the yield strain. Consistent with previous results, the percent relaxation for Med-Spheres (D) is considerably lower than fibers until the strain surpasses their yield strain where total relaxation starkly increases due to spheres shifting and potentially causing microscale fracturing of the granular hydrogel in response to the applied perturbation. Finally, quantifying T_{1/4} for all granular hydrogel groups affirms the previously described differences between fibers and spheres. Across all strains for fiber-based granular hydrogels, there is a generally a modest decrease in T_{1/4} in response to increasing strain. Conversely, Med-Spheres (D) exhibit T_{1/4} values that drastically decrease with increasing strain, then begin to level out once the yield strain is exceeded. These results indicate that yield strain influences the stress relaxation behavior in a strain-dependent manner for the sphere-based granular hydrogels more so than the fiber-based granular hydrogels described here.

### 3.5 Selective Annealing of Fibrous Granular Hydrogels

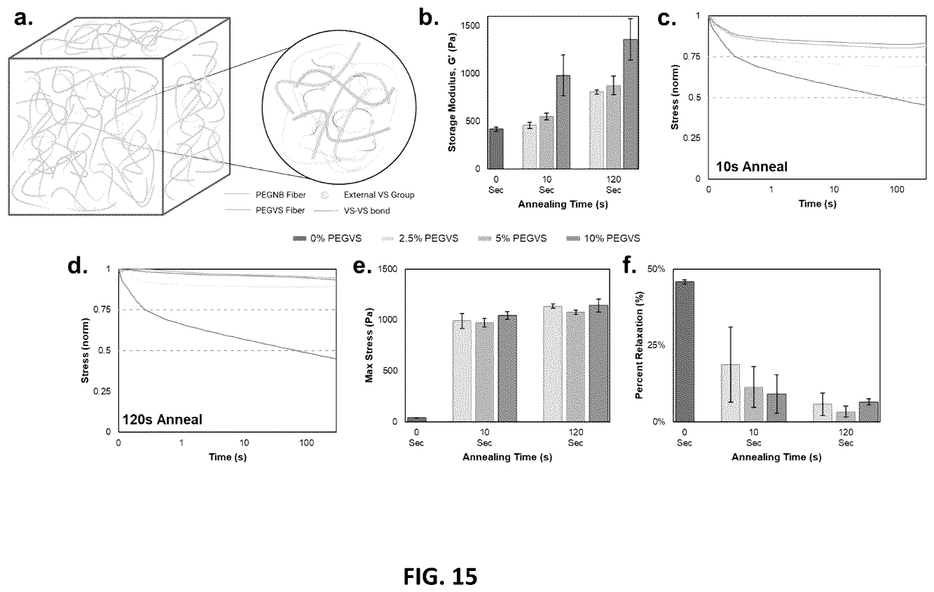

Fiber-based granular hydrogels have thus far demonstrated favorable properties for use as permissive 3D cell culture environments that mimic many properties of natural tissues. They form soft, viscoelastic 3D scaffolds (G′ on the order of 100 Pa) that are mechanically robust. However, most natural tissues are stiffer (G′ on the order of 1+kPa), which might limit their utility in modeling many ECM types. Thus, the mechanical properties of fiber-based granular hydrogels were sought to be increased while potentially maintaining their viscoelasticity and time-dependent stress relaxation properties demonstrated thus far. To achieve this, a PEG-vinyl sulfone (PEGVS) derivative was leveraged to form photoreactive electrospun fibers that could be mixed in with PEGNB fibers are predetermined ratios (PEGVS fibers characterized in FIG. 17). PEGNB fibers were crosslinked with PEGSH in a stoichiometric mismatch, theoretically exhausting all thiol groups—thereby rendering PEGNB fibers theoretically non-photoreactive for further annealing processes. Conversely, PEGVS fibers form kinetic chains during crosslinking which allows crosslinks to propagate when exposed to UV light. This enables a modular system design where the quantity of annealable fibers in fiber-based granular hydrogels can be dictated directly by the ratio of PEGVS:PEGNB fibers in the scaffold (schematic shown in FIG. 15a). The PEGVS form covalent crosslinks between fibers that stabilize the system (contributing to matrix elasticity) and PEGNB fibers are unincorporated in this covalent network, and thus are still able to slide and reorganize around the PEGVS fibers (contributing to matrix viscosity).

PEGVS fibers were added into fiber-based granular hydrogels at 2.5%, 5%, and 10% v/v compared to PEGNB fibers and annealed at 5 mW/cm² for either 10 s or 120 s. All groups exhibited PEGVS content- and annealing time-mediated increases in their respective storage moduli compared to the 0% PEGVS fiber control (FIG. 15b), with the 10% PEGVS groups eclipsing the 1 kPa threshold for both annealing times. Additionally, all groups exhibited both PEGVS content-dependent and annealing time-mediated stress relaxation profiles (FIG. 15c-d). This is grounded conceptually in the formation of kinetic chains when annealing VS groups together. Increasing the volume of PEGVS fibers or the annealing duration enables more crosslinks to form, which increases the magnitude of reinforcement provided by PEGVS fibers in the granular hydrogel scaffold. Therefore, it is aligned with a hypothesis that the lowest quantity of PEGV (2.5% v/v) coupled with only 10 s of UV irradiation yielded the most stress relaxation out of all groups tested. It is noteworthy that any incorporation of PEGVS drastically increased the max stress of the scaffold upon the introduction of 15% strain (FIG. 15e) and also reduced the ability of the granular hydrogel to relax at the global scale compared to the 0% PEGVS group (FIG. 15f). PEGVS fibers theoretically crosslink orthogonally, thereby not covalently interacting with the PEGNB fibers. Thus, PEGVS fibers might provide a covalent scaffolding (i.e., similar to rebar in concrete) that influences mechanical properties at the macroscale, while still enabling the scaffold to exhibit soft, time-dependent stress relaxation properties at the microscale as PEGNB fibers slide and reorganize within and around the annealed structure. Cellular responses at the microscale in scaffolds with varying quantities of annealed PEGVS fibers may be studied.

### 4 Conclusions

Viscoelasticity and stress relaxation are important characteristics of natural tissue that are well-appreciated to influence cell behaviors including migration, proliferation, and differentiation. These complex matrix mechanical properties are often difficult to engineer into traditional 3D bulk hydrogel scaffolds, with granular hydrogels offering some advantages due to their ability to behave like solids, but then reorganize in response to applied forces—features that are analogous to viscoelasticity. This study explores a new class of ECM-mimetic, fiber-based granular hydrogels with tunable viscoelasticity using high aspect ratio (˜15) electrospun PEGNB microfibers. The increased length/diameter ratio enables long-range interactions between discrete fibers which uniquely contribute to complex scaffold mechanics in ways that were previously unrealized by conventional spherical microparticles (aspect ratios of ˜1).

Fiber-based granular hydrogels are viscoelastic with shear-thinning and self-healing capabilities, which are properties consistent with other classes of granular hydrogels. Interestingly, particles with matched volume to the fibers (“Med-Spheres (V)”) are unable to form a scaffolding system with an appreciable storage modulus, indicating that the spherical dimensions to yield the desired volume are insufficient to provide enough contact forces to form a solid-like system at rest. Further, the long-range interactions between individual fibers enable higher yield strains for fiber-based granular hydrogels when compared to spherical-based systems with matched volume and matched dimension. These fiber-fiber entanglements seemingly enable a packing density-dependent stress relaxation profile for fiber-based systems within cell-relevant strain regimes, thereby providing tunability when designing the granular hydrogel system for cell culture applications. Conversely, spherical-based scaffolds are less tunable and exhibit rapid stress relaxation when the applied strain is above their yield strain. Finally, fiber-based granular hydrogels can be selectively annealed via the incorporation of photoreactive PEGVS fibers to increase the macroscale mechanics of the scaffold. It is hypothesized that small amounts (<10%) of PEGVS fibers provides a reinforcing network that penetrates throughout the granular hydrogel, with PEGNB fibers still able to slide and reorganize at the microscale to contribute to the viscoelasticity and stress relaxation that cells might perceive in their environment.

While this study has focused on the mechanical characterization of a novel fiber-based granular hydrogel compared to scaffolds formed from spheres with matched volume and matched dimension, it is important to contextualize this work as a 3D, permissive cell culture scaffold. Perhaps the most notable property of fiber-based granular hydrogels is their tunable stress relaxation that is largely independent from yield strain, with T_{1/2} values ranging from 1-100+s, depending on packing density. These timescales are physiologically relevant for many tissue types, thereby offering user-defined design control over the time-dependent mechanics of the tissue culture scaffold. Additionally, the subcellular length scale diameters of these PEGNB fibers might offer a more permissive granular hydrogel environment compared to spherical particles that are commonly sized to be on the same order of magnitude as cells, or larger—possibly providing the ability for cells to navigate their environment without hindrances introduced by the particles. These properties are analogous to those in many natural tissues where a soft, viscoelastic hydrogel occupies most of the space in the ECM, and mechanical properties in the fiber-based granular hydrogels are demonstrated to support this. These advantages may lend themselves to modeling tissue systems—like brain tissue—where the amorphous hydrogel dominates the mechanical properties of the environment and cells are known to migrate and grow independently of adhesion forces.

Many natural tissue types also contain protein fibers that mechanically strengthen the amorphous, viscoelastic hydrogel that occupies the ECM. This inspired the inclusion of PEGVS fibers within the PEGNB fiber-based granular hydrogel to covalently stabilize the network. In addition to the mechanical support offered by the annealed PEGVS fibers, they might provide immobilized anchoring points for cells to engage with and exert traction forces like they would in many endogenous tissue environments. Importantly, both PEGNB fibers and PEGVS fibers are readily modifiable with protein-mimetic and adhesive peptide ligands to increase the bioactivity of PEG to support cell engagement with the granular hydrogel network.

Overall, fiber-based granular hydrogels offer an intriguing alternative to traditional granular hydrogels comprised of spherical microparticles. Their mechanical tunability to match complex matrix properties of different tissue types offers a promising solution to engineering 3D scaffolds for cell culture applications. While the focus of this study was to characterize their range of physical properties, this new class of granular hydrogels is expected to lead to further exploration into their utility as a cell culture scaffold for both in vitro and in vivo applications.

### Example 3

Particle-based (granular) hydrogels are an attractive class of biomaterials due to their unique properties and array of applications in the biomedical space—serving as platforms for extrusion printing and injecting as well as permissive materials for 3D cell culture. Physical properties of particle-based hydrogels are governed in part by contact forces between particles, which are limited to interactions with neighboring particles. Secondary annealing mechanisms are often used to increase mechanical properties and serve to link particles across the granular material volume. Herein, a novel particle-based hydrogel where each “particle” is a discrete electrospun hydrogel microfiber that has been segmented to a length of 93±51 μm, with a diameter of 1.6±0.3 μm are presented. The fibers are flexible and have aspect ratios that are greater than one order of magnitude larger than most traditional hydrogel microparticles. This enables long-range entanglements of discrete fibers following packing into a bulk material, yielding unique properties. Without crosslinking, these packed hydrogel microfiber materials are mechanically robust, they can stretch without breaking when strained, and they exhibit stress relaxation under constant strain. As a cell culture scaffold, shear-induced alignment of the individual fibers within 3D printed filaments confers contact guidance cues to cells and promotes anisotropic cellular morphologies. Packed hydrogel microfibers can also be used as 3D cell culture environments, with cells able to spread due to the permissive nature of the scaffold. Overall, this work introduces a particle-based material system comprised of individual hydrogel microfibers that allows unique properties to be engineered into biomaterials that might be used in extrusion processes and cell cultures and, ultimately, in tissue engineering and regenerative medicine applications.

### 1 Introduction

In recent years, granular hydrogels—which are hydrogel materials comprised largely of discrete hydrogel microparticles (HMPs) held in place by particle-particle contact forces and, often, engineered interparticle interactions—have received increasing attention in biomaterials research. Granular hydrogels are attractive for reasons that include properties that enable injection delivery as well as control over mechanics and degree of porosity. Discrete HMPs, often spherical particles formed through microfluidic or emulsion approaches (on the order of 10¹-10² μm in diameter), are packed to yield a macroscale construct held together by physical (e.g., contact) or chemical (e.g., covalent) interactions, or both. Physical interactions are commonly introduced through packing of individual HMPs via centrifugation or vacuum filtration where interstitial fluid between the particles is largely removed. This places HMPs in direct physical contact where particle-particle contact interactions determine mechanical properties of the granular hydrogel as a whole. These purely physical interparticle forces allow for the granular hydrogel to be stable at rest, but individual particles will begin to slide and flow when a force is applied that overwhelms contact interactions in the system (e.g., during extrusion).

Chemical crosslinking, or annealing, between particles can improve mechanical stability in granular hydrogel systems. Annealing can be employ covalent or supramolecular crosslinks between reactive moieties on the surface of discrete particles to stabilize the granular hydrogel. In injection applications into a tissue defect or wound, this stabilization has enabled a class of granular hydrogels known as microporous annealed particle (MAP) scaffolds to serve as injectable tissue regeneration platforms. There has been considerable work tuning HMP properties, and therefore granular hydrogel behaviors, by varying crosslinking and secondary annealing mechanisms, introducing degradability, and using HMPs to deliver bioactive molecules post-delivery. This degree of specificity over individual particles allows for engineered modularity and control over physical and chemical heterogeneity in granular hydrogel materials.

Advanced strategies to tune the biomimicry of individual HMPs are largely predicated on spherical particles (aspect ratio˜1). Shifting away from spheres, studies leveraging particles with increased aspect ratios have elucidated some unique characteristics when assembled together into granular hydrogel scaffolds. For example, rod-shaped HMPs (aspect ratios ranging from ˜2-20) enable larger, more interconnected pores throughout the scaffold, which facilitate greater cell migration and infiltration. Increasing the size of HMPs while conserving the higher aspect ratios, leads to long, flexible hydrogel strands that can align and entangle when assembled into a granular hydrogel—offering interactions at increased length scales compared to other, lower aspect ratio HMPs. These entanglements are useful for extrusion mechanisms (e.g., injection and 3D printing) and also enable increased granular hydrogel structural fidelity without secondary annealing.

Decreasing the diameter of these high aspect ratio strands to sub-cellular length scales (˜1 μm diameter “fibers”) offers a granular hydrogel platform where cells are able to recruit individual fibers and reorganize the structure of the scaffold. While these have been densely assembled and immobilized through designed annealing interactions, development of a materials strategy using dense combinations of hydrogel fibers without secondary annealing mechanisms to yield granular hydrogels with high mechanical stability and high degrees of permissivity was pursued. To achieve this, hydrogel microfibers are electrospun and segmented to yield discrete “particles” with sub-cellular length scale diameters and high aspect ratios, which are analogous to extracellular matrix (ECM) fibrous proteins like collagen. These individual microfiber segments represent the “grains” that are packed through centrifugation to form the granular hydrogel scaffold—hereafter denoted as packed hydrogel microfiber (PHM) scaffolds. Herein, PHM scaffolds allow the design of materials which are strain yielding and while exhibiting unique stretching properties. Furthermore, these materials dissipate stress in response to applied strains similar to biological materials and maintain their mechanics with increasing interstitial fluid within the system (i.e., decreasing packing density). Finally, PHM scaffolds can influence cell behaviors through topographical cues, which can be dictated via extrusion and bioprinting, as well as through their unique physical properties as 3D, permissive cell culture scaffolds.

### 2 Results and Discussion

### 2.1 Preparing Packed Hydrogel Microfiber Scaffolds

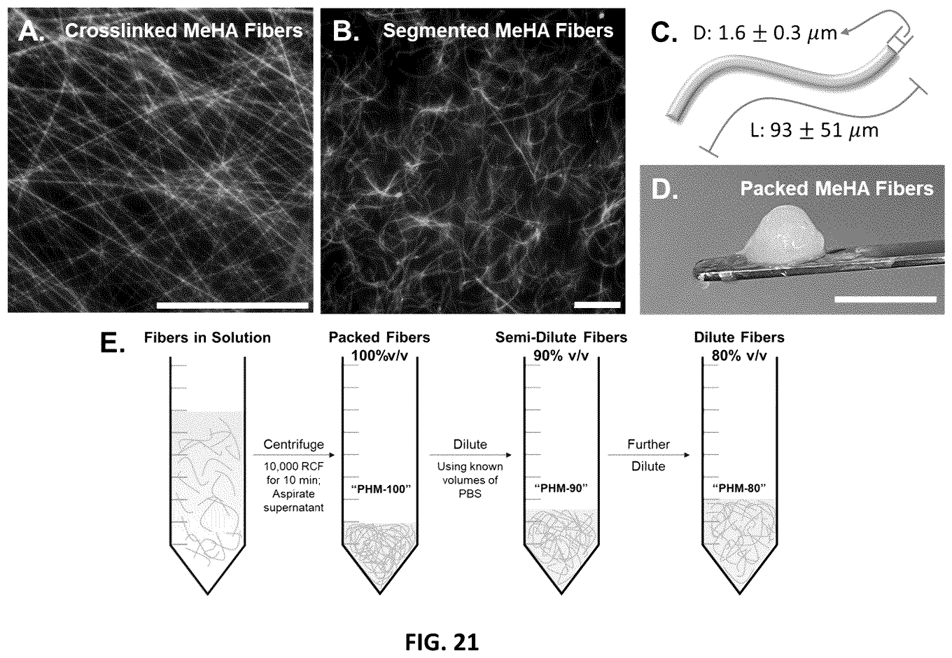

Packed hydrogel microfibers were fabricated from electrospun methacrylated hyaluronic acid (MeHA), which has demonstrated biocompatibility and been previously used in electrospinning. MeHA synthesis was confirmed by ¹H NMR spectrum. Electrospun MeHA fibers were designed to model endogenous ECMs both through the use of a material based on the native glycosaminoglycan, hyaluronic acid (HA), and its subsequent processing into hydrogel microfibers that mimic the natural protein fibers in the ECM. Methacrylation enabled photomediated crosslinking to stabilize the fibers prior to hydration (FIG. 21A) and, through reactive methacrylates that remain after photocrosslinking, coupling of the fibronectin-mimetic Arg-Gly-Asp (RGD) adhesive motif through a Michael addition reaction to a cysteine residue incorporated into the RGD-containing peptide.

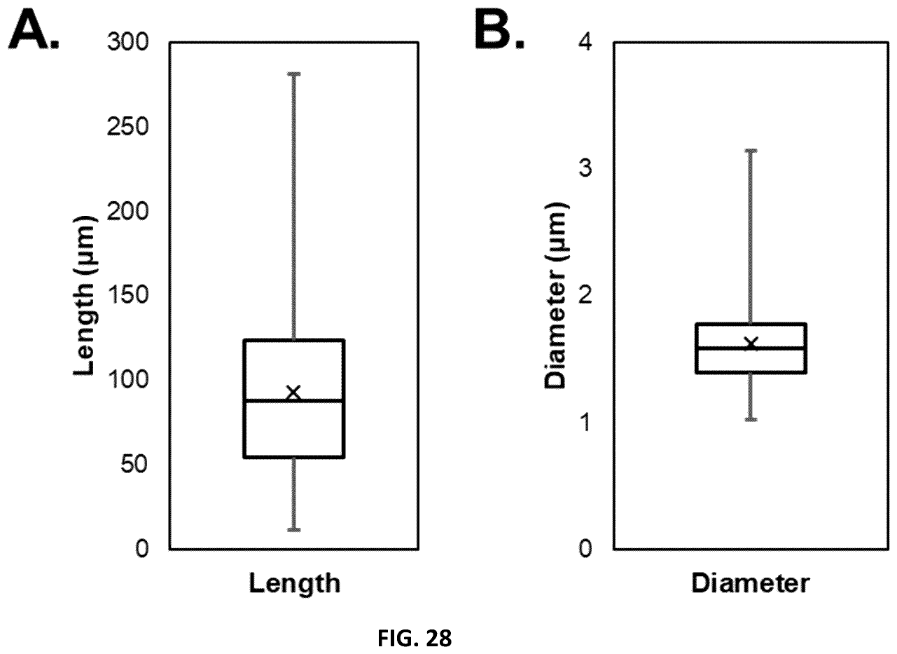

To create segmented microfibers that can be packed together to form PHMs, crosslinked MeHA fibers were hydrated then segmented via a series of triturations through needles of decreasing inner diameter (16 G, 18 G, and 20 G). The resultant fiber segments (FIG. 21B) were 1.6±0.3 μm in diameter and 93±51 μm in length (FIGS. 21C and 28). After packing the suspension of discrete fibers by centrifugation, they behaved as a bulk solid at rest (FIG. 21D), similar to conventional granular hydrogels. Compared to granular materials based on spherical particles, the high length:diameter aspect ratio of the microfibers in a PHM scaffold allows for unique long-range entanglements. These long-range interactions allow for dilution of the “fully packed” scaffolds to increase inter-fiber fluid content and provide more space between individual fiber segments. Here, “fully packed” is defined as scaffolds as consisting of 100% v/v of the material recovered from centrifugation. The fully packed material, hereafter referred to as “PHM-100”, can be further diluted with known volumes of PBS to 90% v/v and 80% v/v, referred to as “PHM-90” and “PHM-80”, respectively (shown in FIG. 21E).

### 2.2 Mechanical Characterization of PHMs

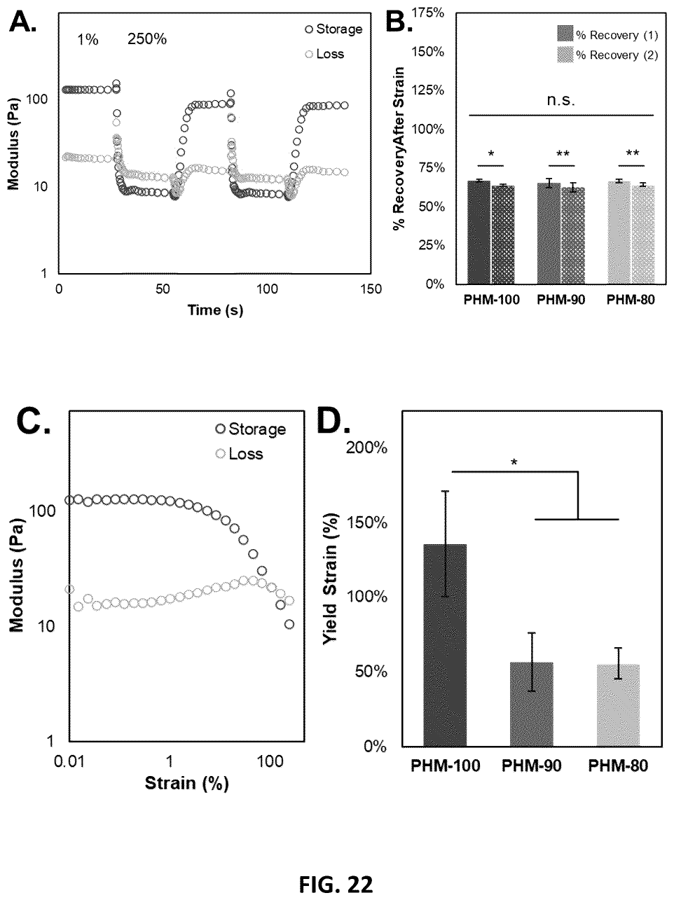

PHM-100, PHM-90, and PHM-80 all exhibit characteristics that are important for particle-based hydrogels used for extrusion processes or in cell culture systems. Shear-thinning and self-healing were evidenced in all dilutions via oscillatory shear rheology (FIG. 22A) through repeated cycling between low (1%) and high (250%) strains. In contrast to granular hydrogels based on spherical particles, the storage modulus recovered after removal of the high strain from the system was reduced (˜60-70%) in all groups (FIG. 22B). This reduction was statistically significant and observed between the first and second low-strain regimes, with no statistical significance in the percentage drop in modulus across dilutions. PHM behavior is attributed to rearrangements of microfibers during rheometric analysis. After initial packing via centrifugation, the organization of the segmented fibers is likely maximally entangled through random organization, giving rise to the initial mechanical properties of the scaffold. During the high-strain regimes, this organization is disrupted, and a portion of the long-range entanglements is irreversibly lost.

As in other granular hydrogel and shear thinning systems, the ability to convert from a solid to liquid-like state above a yield strain (defined as the crossover point between G′ and G″ in a strain sweep, FIG. 22C) allows the solid-like material to be injected or extruded. The 136% yield strain (FIG. 22D) for the 100% v/v group (PHM-100) is notably larger than other granular hydrogel systems. Diluted fiber density in the PHM-90 and PHM-80 groups yielded statistically decreased yield strains of 56% and 57%, respectively, which is consistent with yield strains of other reported granular hydrogel systems. These data indicate that all test groups are shear-thinning and self-healing, with yield strains generally in ranges characteristic of granular hydrogels used in extrusion processes. Furthermore, increased yield strains are possible in PHM formulations, which indicate the potential for enhancing the stability of particle-based hydrogels through dense fiber-based formulations.

### 2.3 Evaluating the Extensibility of PHM Scaffolds

Anticipating that long-range interactions between microfibers in PHM scaffolds resulting from their high aspect ratios would offer unique bulk properties, the extensibility of the PHM-based materials was examined. A modified version of filament stretching extensional rheology (FiSER) was conducted, in which a material can be stretched as a filament, allowing strain to failure to be observed as a measure of material extensibility or stretchability. It is hypothesized that discrete fibers within the filament would participate in interactions at extended length scales compared to conventional granular materials, thus resulting in highly extensible PHM materials when stretched. Compared to spherical particle interactions, which would be restricted to engagements with a limited number of neighboring particles, a PHM scaffold would stretch more and appear less brittle as a bulk than a conventional granular hydrogel.

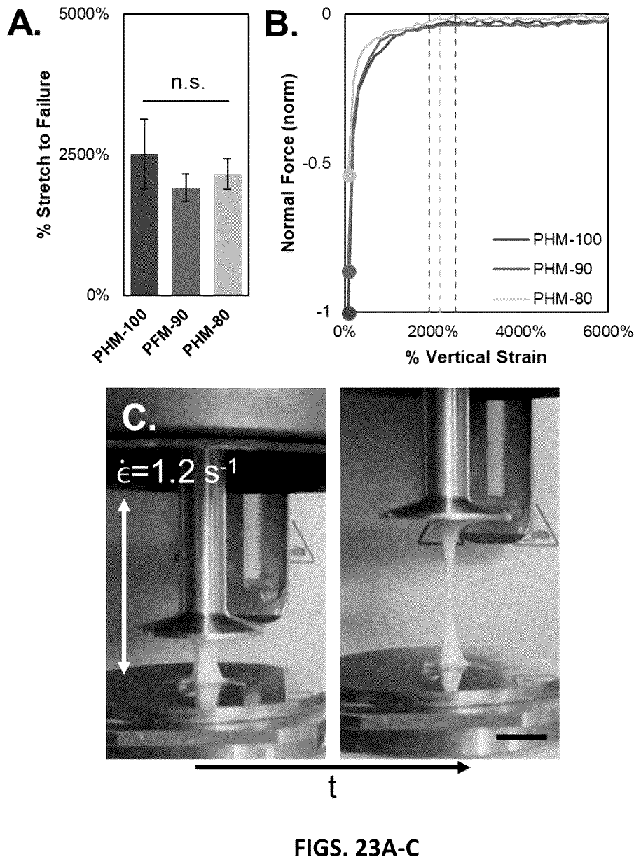

In modified FiSER testing, all groups of PHMs exhibited strain-to-break values of 2000-2500% (FIG. 23A), indicating fibers maintain filament-stabilizing interactions in response to stretching. Notably, while PHM-100 exhibited the highest degree of stretchability (˜2500%), there was no statistical difference between the groups. In these measurements, the normal forces sustained by the filaments during extension (FIG. 23B) were observed to decrease with respect to hydrogel microfiber density. These observations combine to suggest that the extensibility of a PHM scaffold is dictated by fiber geometry, while the density of fiber-fiber interactions (i.e., the combination of entanglements and surface-surface interactions) drives the forces that can be sustained during stretching. Correspondingly, the normal force profiles for each group exhibit the same trend as they are stretched to failure, which occurs at % strains that exhibit no statistical difference. This extensibility (visualized during testing of PHM-100 in FIG. 23C) is believed to be unique among particle-based hydrogels where there is no dissolved polymer between particles, and that it is driven by enhanced interactions among the discrete elements of the bulk material that result from the high-aspect ratios of the individual fibers in the PHM scaffold.

### 2.4 Characterizing Viscoelasticity and Stress Relaxation of PHM Scaffolds

Given observation of these dynamic behaviors of PHM hydrogels, further characterizing their viscoelasticity and stress relaxation to determine whether they might offer new properties as cell and tissue culture scaffolds was of interest. Endogenous tissue exhibits a host of complex, time-dependent mechanical properties that are difficult to recapitulate in traditional hydrogel materials. For example, ECM structural components like collagen fibers present in many native tissue microenvironments enable the dissipation of stress over time, thereby contributing to nonlinear viscoelasticity profiles. Inspired by this and other long-standing observations of the similarities between the geometries of electrospun fibers and ECM fibrous proteins, it is hypothesized that noncovalent interactions between individual fibers in 3D PHM materials would allow effective mimicking of the stress relaxation of native ECM in a synthetic system. As noted above, the previous characterization experiments suggested the ability of fibers within the PHM hydrogels to interact at rest but slide past each other and reorganize to dissipate forces applied to the materials.

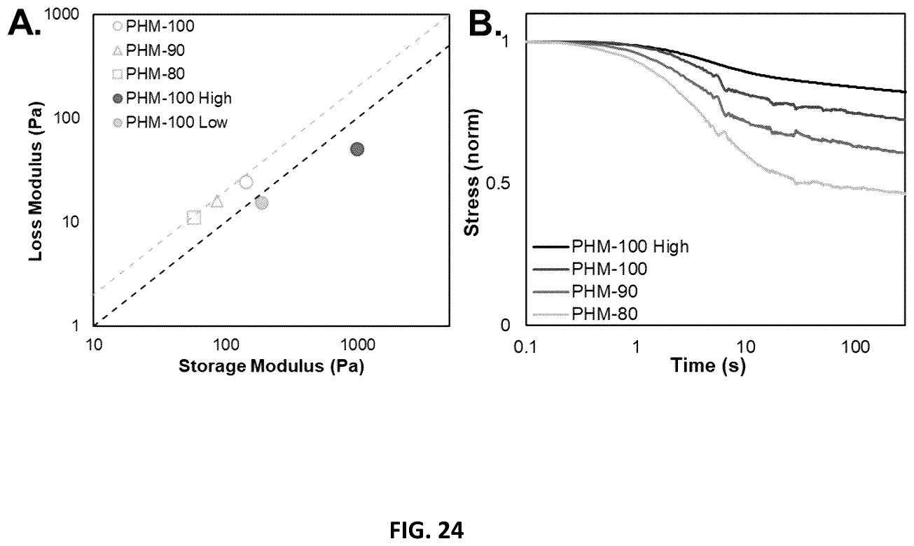

To assess PHM scaffold viscoelasticity for comparison to biological tissues and materials, the loss modulus (i.e., viscous component, G″) was measured versus storage modulus (i.e., elastic component, G′) from rheometric time sweeps (FIG. 24A). All PHM formulations exhibited relatively soft bulk storage moduli—ranging from ˜50 Pa for PHM-80 to ˜150 Pa for PHM-100, despite individual hydrogel microfibers having moduli many orders of magnitude greater than bulk fiber-based scaffolds. Additionally, all groups demonstrated viscoelastic behavior with their storage moduli approximately 5× their loss moduli (grey dashed line in FIG. 24A illustrates where G′=5×G″). Importantly, endogenous tissue typically possesses a higher elastic contribution, where the storage moduli are typically 10× the loss moduli. It is postulated that this 10× target could be achieved via additional interfiber crosslinking (or annealing) of PHMs.

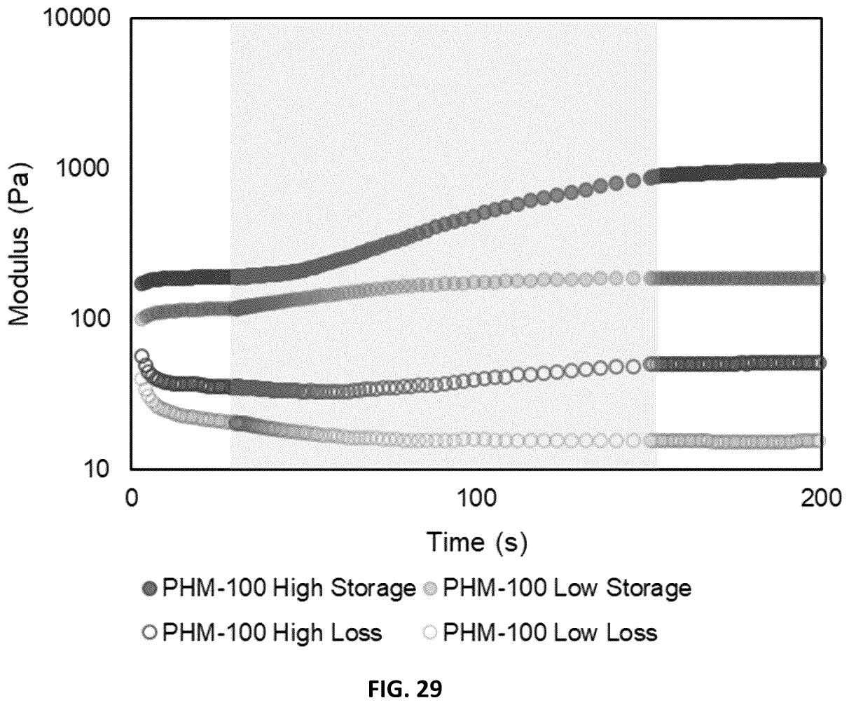

To test this, PHMs were designed to have residual methacrylate groups on the surfaces of individual microfibers that were not consumed during the initial fiber crosslinking process. Utilizing the PHM-100 formulation, “High” and “Low” crosslinkable fibers, were created where the “PHM-100 High” group was treated after fiber segmentation to remove some reactive methacrylate groups (to mimic RGD modification) but had most residual methacrylate groups available for crosslinking. The “PHM-100 Low” group was similarly treated after fiber segmentation to eliminate most, but not all, residual methacrylate groups on the fibers. Therefore, PHM-100 High could achieve considerable annealing during a secondary crosslinking step by inducing methacrylate polymerization. Conversely, PHM-100 Low could undergo only a comparatively low degree of secondary crosslinking, resulting in reduced interfiber annealing compared to PHM-100 High (rheology for the secondary UV-initiated annealing step shown in FIG. 29).

It was observed in both groups (PHM-100 High and PHM-100 Low) that interfiber crosslinking shifted the G′:G″ ratio towards 10:1 (FIG. 24A, shaded circles, dashed black line represents G′=10×G″). PHM-100 Low exhibited a stark decrease in G″ coupled with a marginal increase in G′ compared to PHM-100. Interestingly, PHM-100 High exhibits a slight increase in the loss modulus coupled with a notable increase in the storage modulus, which also shifted the viscoelastic moduli toward the 10:1 ratio that characterizes many biological tissues. Taken together, these data suggest that the degree of secondary crosslinking afforded by available methacrylate groups can be utilized to controllably modulate the viscoelasticity of PHM hydrogel systems.

As previous tests pointed to dynamic responses to applied stress in PHM scaffolds, their capacity to undergo stress relaxation was evaluated. Time-dependent stress relaxation is a feature of many biological material systems that is observed to critically influence cellular behaviors; however, it is challenging to engineer and control stress relaxation in many hydrogel systems. In this analysis, a constant shear strain of 15% was applied to the sample and recorded the resultant stress as a function of time to assess how the material relaxes in response to the applied strain. Plotted as the normalized stress in FIG. 24B, PHM-100, PHM-90, and PHM-80 all exhibit varying degrees of stress relaxation, with increasing relaxation corresponding to increased dilution with PBS. The enhanced relaxation for the diluted groups is attributed to more space for fibers to reorganize in response to the applied strain and reduced interactions between fibers on a per volume basis. PHM-100 experiences the highest degree of these interactions and the most confining interfiber space that slows or prevents fiber movement that results in PHM stress relaxation.

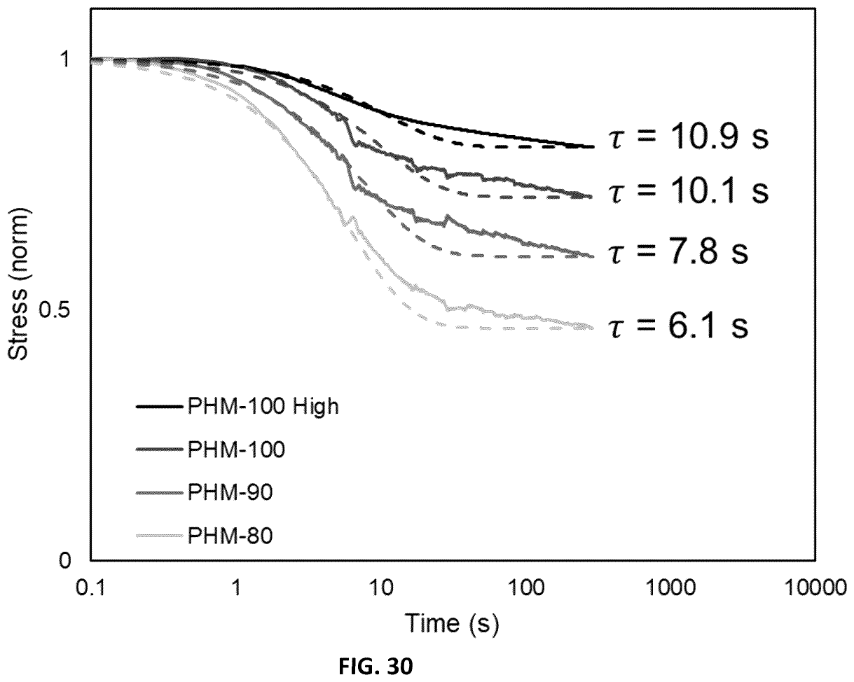

To observe the effects of interfiber crosslinking, the same 15% strain was applied to the PHM-100 High group. PHM-100 High dissipated stress modestly (FIG. 24B). However, the covalent interfiber crosslinking restricted the ability of the microfibers to move and thus prevented the material from relaxing to the extent of the non-crosslinked groups (FIG. 24B). Importantly, with respect to recapitulating the relaxation time scales of viscoelastic solids, the relaxation profiles of the PHM hydrogels began to plateau within 100 s of stress being applied. Through the use of a viscoelastic standard linear solid model (FIG. 30), characteristic relaxation times (τ) were calculated. All groups, including the PHM-100 High crosslinked material, exhibited most of their stress relaxation within a characteristic time of approximately 5-10 s, indicating that PHM systems respond to applied strains rapidly. From these data, these non-crosslinked materials are expected to be useful in engineering soft tissue systems in vitro, and in engineering both soft and stiffer tissues (via secondary crosslinking) to model physiological systems where ˜100 s and quicker relaxation times are desired.

### 2.5 Extrusion of PHM Inks

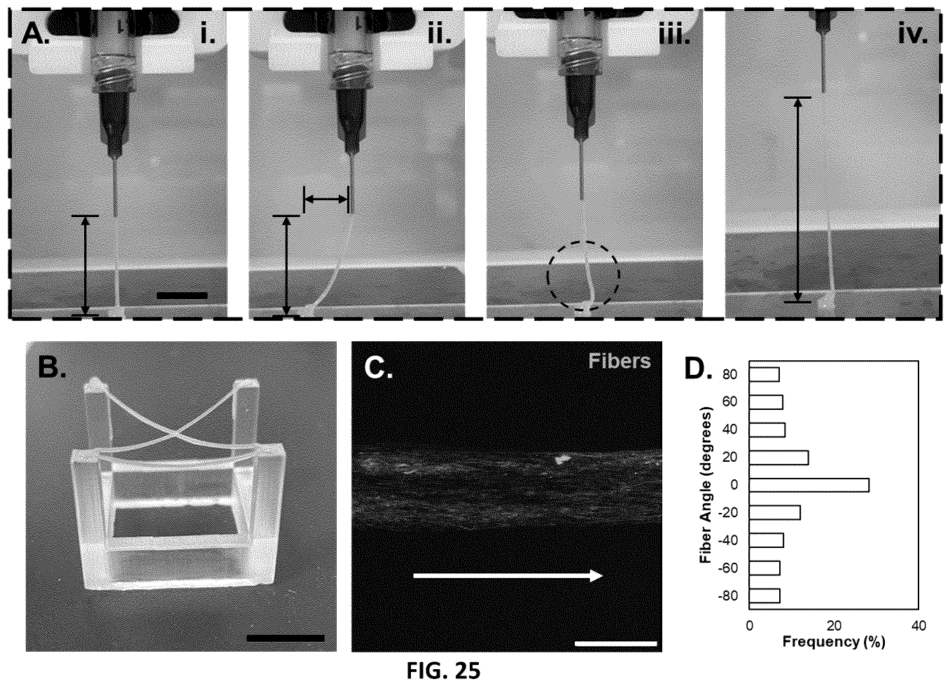

As mentioned, the rheological and mechanical properties of PHM materials should enable diverse uses in applications where injectable or extrudable biomaterials are desired, including in biomedical applications of 3D printing. The responses to extrusion processes using a 3D printer (FELIX BIOPrinter) were studied. In controlled extrusions, a 2 cm vertical filament of PHM-100 was printed through a 22 G needle (ID: 0.413 mm). This yielded a filament with a diameter of ˜0.5 mm (FIG. 25Ai). Towards observing the stability of PHM filament without interfiber crosslinking (a filament stabilized strictly by noncovalent interactions), the vertical filament was translated 1 cm horizontally without further extrusion (FIG. 25Aii) and then back to the original position (FIG. 25Aiii). The filament was easily manipulated without breakage. Additionally, the filament stretched noticeably as a result of undergoing dynamic stress relaxation when extended without extrusion (FIG. 25Aiii, dashed circle). To further observe the extent to which the filament was extensible, the nozzle was moved vertically an additional 2 cm without extrusion. This high degree of extension is attributed to the long-range interactions among the individual microfibers, which provide additional stability in the filament that would not occur with other particle-based systems with smaller aspect ratios.

Finally, towards demonstrating the remarkable stability of the non-covalently annealed PHM filaments, filaments were extruded horizontally across the posts of a 2.5×2.5 cm inverted table (FIG. 25B). The resultant filaments were mechanically stable, spanning these gaps without breaking while stabilized only by physical interparticle interactions. This demonstration of the strength of long-range interactions among individual fibers being sufficient to maintain filament integrity at longer scales is an exciting feature of the PHMs as a granular hydrogel system. These properties may have value in numerous applications that use extrusion processes, including 3D printing, where structural fidelity of printed hydrogel inks remains a central consideration in developing new biomaterial inks.

In addition to observing these macroscale characteristics of extruded filaments, the effects of extrusion processing on the PHM filaments at the microscale, in particular the organization of the fibers after extrusion were of interest. From the previous rheological studies, the organization of fibers within PHM scaffolds are concluded to drive mechanical properties but can be influenced by high shear. Therefore, it is hypothesized that the shear introduced by the extrusion process might disrupt fiber orientation and induce anisotropic alignment in the direction of shear. To assess this, nonfluorescent fibers were mixed with fluorophore-tagged fibers at a 10:1 ratio to enable visualization of the extruded material, and printed PHM-100 onto glass coverslips. Indeed, fibers aligned in the direction of the applied shear (FIG. 25C-D). This result follows previously demonstrated shear-induced alignment of fibers embedded in bulk gels by Prendergast and coworkers, as well as alignment demonstrated by Kessel et al. using hydrogel microstrands and Sather et al. utilizing self-assembled supramolecular nanofibers. However, in the PHM materials used here, which consisted entirely of electrospun fibers with diameters on the order of 1 μm, there was a unique opportunity to directly control the surface topography to direct cell behaviors. The importance of microscale topography and contact guidance on cellular behaviors has been well studied and characterized, and the ability to dictate surface topography via extrusion using PHMs is an exciting opportunity to extend work using electrospun hydrogel fibers.

### 2.6 PHM Scaffolds Support 2D and 3D Cell Culture

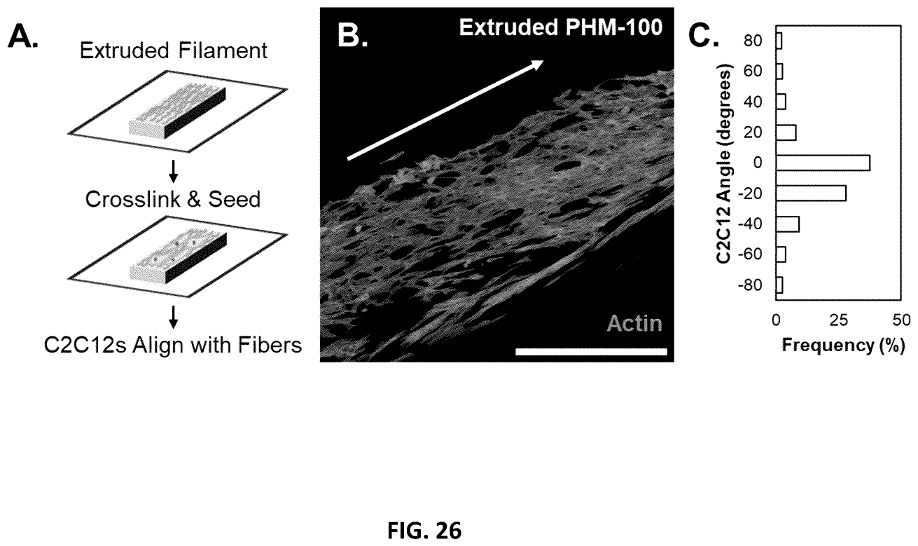

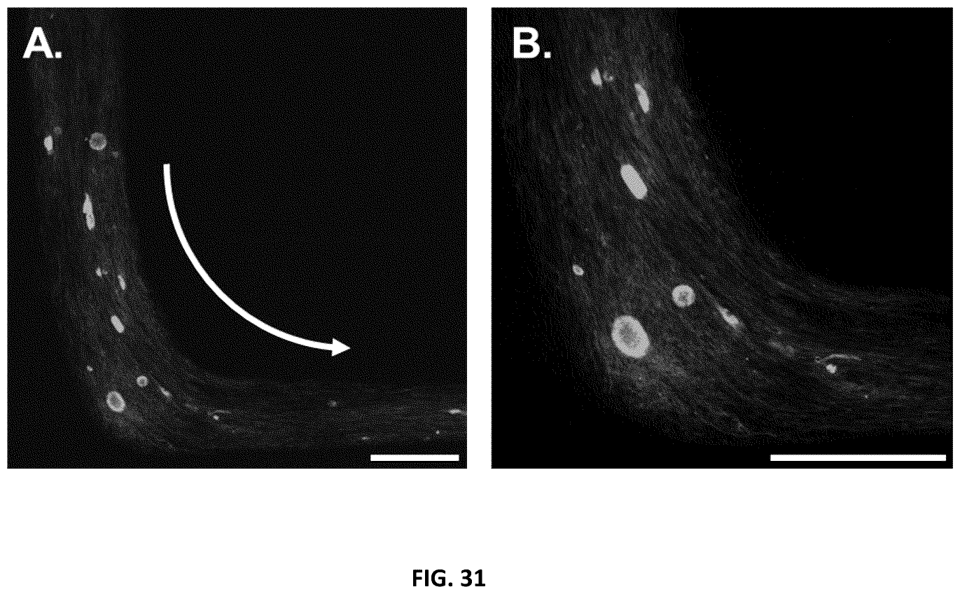

Given the potential to use PHM materials in bioprinting to create materials with specified surface anisotropies and to design permissive 3D environments using PHMs, cellular responses to these materials were examined next. To assess whether fiber alignment in filaments would provide microscale topographical cues to cells that interacted with these materials, a PHM biomaterial ink was printed and cells were seeded onto it. PHM-100 was extruded with 1 mM RGD (analogous to PHM-100 High) that could undergo a secondary interfiber crosslinking onto glass coverslips and then irradiated with light to stabilize the filament through secondary crosslinking. Immortalized murine myoblasts (C2C12 s), which are known to respond to alignment cues, were seeded atop the crosslinked PHM-100 filament (FIG. 26A). Following a 2d culture period, cytoskeletal staining showed that cells aligned with the direction of microfibers in the filament (FIG. 26B-C), indicating that the microscale topography provided contact guidance that can influence cellular organization. Because fiber alignment responds to changes in needle direction (FIG. 31), these results suggest the ability to influence cell directionality via the needle path during extrusion, with arbitrary 2D topography and cell alignment defined by the extrusion printing process when using PHMs as a biomaterial ink.

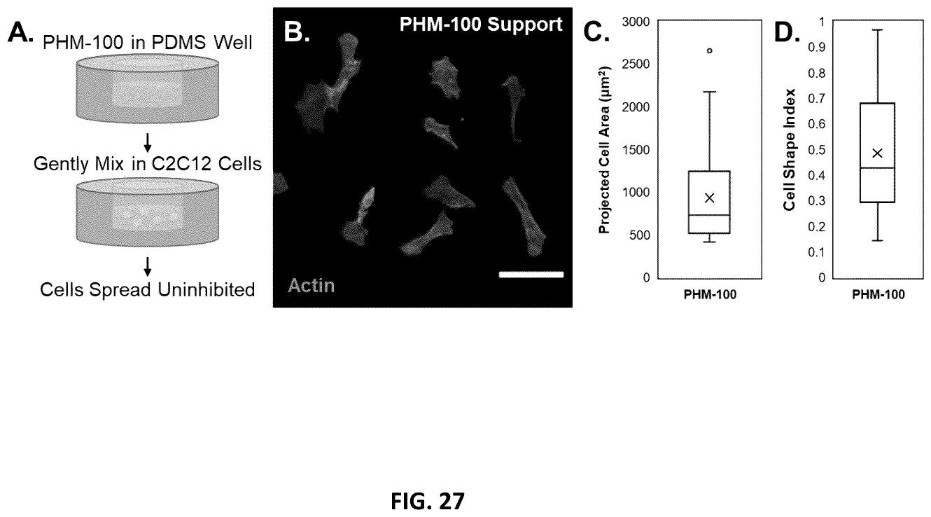

Further studies were conducted to investigate how cells within a 3D PHM-based hydrogel would behave, given the dynamic properties measured previously. Since the most fiber-dense formulation (PHM-100) is soft and viscoelastic, C2C12 s may be able to reorganize the constituent fibers during proliferation, migration, and interactions with the environment. Over short time scales, cell spreading within these materials was expected as opposed to rounded morphologies typical of cells in 3D hydrogels that are not permissive. To investigate this, C2C12 s were gently mixed with PHM-100 which would undergo no interfiber crosslinking, in order to maintain the dynamic properties observed in previous experimentation. The C2C12+PHM-100 was then placed into a PDMS mold and covered with 100 μm pore filter paper to prevent PHM-100 from disassociating in culture media (FIG. 27A). After 1 d in culture, cytoskeletal staining showed that C2C12 s were able to spread freely in PHM-100 (FIG. 27B), with a wide range of projected cell areas, along with a loss of circularity (cell shape index) that would be expected in a covalently crosslinked hydrogel quantified in FIG. 27C-D.

These observations suggest that PHM-100, even absent interfiber crosslinking, supports 3D cellular activity. The cell spreading observed was in a most fiber-dense formulation, suggesting that PHMs yield in response to cells' movements and impose minimal spatial restrictions on the cells. Because the microfiber elements of the material are soft and have diameters on the order of 1 μm, they may be uniquely permissive to cellular activity. In comparison to larger spherical particles used in other granular materials, whose diameters are on the order of 10¹-10² μm, PHMs may present an alternative physical environment to the cells incorporated into them in vitro or with cells that engage with them when applied in vivo. Opportunities to reorganize and move throughout 3D space exist in PHM-type systems that may be less accessible in granular materials with larger particle diameters, where particle movement in response to cellular activity would be limited, resulting in cells negotiating the surfaces of the particles and the spaces in between. PHMs offer exciting new opportunities within particle-based materials through their presentation of robust bulk properties emerging from a microenvironment comprised of individual fibers that cells can readily interrogate, reorganize, and migrate around.

### 3 Conclusions

In summary, a new class of particle-based hydrogels comprised solely of discrete electrospun microfibers have been developed. Packed hydrogel microfiber scaffolds (PHMs) are shear-thinning and self-healing, with strain yielding responses that may enable application in extrusion or injection processes. The high-aspect ratios of the fibers and long-range entanglements provide physical interactions in PHMs that result in robust materials that behave elastically as a bulk below a yield strain. These interactions also enable high degrees of extensibility in packed scaffolds. PHMs can be stretched to greater than 2000% of their original height; a phenomenon that is conserved across PHM scaffolds during dilution. PHM scaffolds are also viscoelastic and quickly (˜10 s) dissipate stresses applied to them. Macroscale extrusion printing demonstrates filament fidelity and robust stability, even without secondary crosslinking to stabilize filaments. Shear-induced alignment of component microfibers during extrusion can be leveraged to direct cellular alignment when seeded on top of printed filaments. Cells seeded within these materials—or potentially infiltrating into these materials—experience permissive, 3D environments that they can interrogate and possibly remodel during migration and proliferation.

The novel packed hydrogel microfiber system as described herein may provide an exciting new avenue for designing granular hydrogel media. The flexibility afforded by the PHM preparation process coupled with the ability to tailor individual fibers that comprise the bulk scaffold may enable new approaches to engineering synthetic models of the ECM in vitro and new opportunities for engineering implantable biomaterials.

### 4 Experimental Section/Methods

All reagents were purchased from Millipore Sigma, unless otherwise stated.

4.1 Methacrylated hyaluronic acid (MeHA) synthesis: Hyaluronic acid (HA) was functionalized with methacrylates as previously discussed. Briefly, sodium hyaluronate (Lifecore, 60 kDa) was dissolved in deionized water at 2% w/v. While maintaining the solution at a pH of 8.5-9, methacrylic anhydride (Sigma Aldrich, 4.83 mL per g HA) was added dropwise to the solution. The reaction mixture was maintained at a pH of 8.5-9 for 6 h on ice, then continued to react at room temperature overnight. The reaction was dialyzed against deionized water (SpectraPor, 6-8 kDa molecular weight cutoff) at room temperature for 5 days, then frozen and lyophilized to dryness. The final methacrylate functionalization was 100% by quantification with ¹H NMR (500 MHz Varian Inova 500).

4.2 Electrospinning MeHA microfibers: To electrospin MeHA, solutions consisting of 3% w/v MeHA, 2.5% w/v polyethylene oxide (900 kDa), and 0.05% w/v 2-hydroxy-4′-(2-hydroxyethoxy)-2-methylpropiophenone (HHMP) were mixed overnight in DI H₂O. To fluorescently tag microfibers for characterization and visualization, 0.4% w/v fluorescein isothiocyanate-dextran (FITC-dextran) was included in the electrospinning solution. The MeHA solution was extruded through a 16-gauge needle at a rate of 0.5 ml h⁻¹ with an applied voltage of 12-14 kV. Microfibers were collected on a negatively charged (−4 kV) rotating mandrel (DOXA Microfluidics) moving at a linear velocity of 10 m s⁻¹. Fiber batches were collected for 1 h before a 2 min UV crosslinking step (365 nm, 5 mW cm⁻², VWR UV Crosslinker) to stabilize fibers for subsequent segmentation steps.

4.3 Preparation of PHMs: Fibers were hydrated in PBS for at least 1 h and then comminuted through a series of extrusion steps to yield small fiber segments. Beginning with a 16-gauge needle, the fiber solution was passed up and down the needle 25× to preliminarily break up the fibers. This process was repeated with an 18-gauge needle, and finally a 20-gauge needle to yield the final fiber segments. The resulting solution was then centrifuged, the supernatant was discarded to remove the PEO and unreacted HHMP, and the fibers were resuspended in a known volume of PBS to yield a stock concentration of 10% v/v. Fiber stock solutions were stored at 4° C. until further use. Fluorescently tagged MeHA fibers were diluted and imaged on a Leica DMi8 widefield fluorescence microscope to characterize fiber diameter and length (n>150 fibers) post-segmentation.

For cell culture assays, fibers were functionalized with a fibronectin-mimetic Arg-Gly-Asp (RGD)-containing adhesive peptide (GCGYGRGDSPG, Genscript) via the thiol-Michael addition reaction to promote integrin-mediated cellular interactions in the scaffold. Briefly, fibers were suspended at 10% v/v in the presence of 1 mM RGD, the pH of the solution was elevated to 8 using triethanolamine and allowed to react for 2 h at 37° C. Fiber solutions were centrifuged and resuspended in PBS thrice to removed unreacted molecules prior to packing.

Fiber solutions were packed (condensed into a minimal volume with minimal solvent between the fibers) via centrifugation at 10,000 RCF for 10 min, with all remaining supernatant decanted to yield the shear-thinning and self-healing PHMs used within this work. PHMs were either used as is (denoted as PHM-100), or further diluted with known volumes of PBS to yield 90% v/v and 80% v/v (denoted as PHM-90 and PHM-80) relative to the original packing density to increase interstitial space between fibers.

4.4 Preparation of crosslinkable PHMs: For low crosslinking, fibers were treated with 250 mM cysteamine at pH 8 for 2 h at 37° C. to quench most pendant methacrylate groups and prevent significant crosslinking. For high crosslinking, the fibers were treated with 1 mM cysteamine at pH 8 for 2 h at 37° C. to mimic the RGD addition scheme, while leaving most pendant methacrylates free. Crosslinked fiber scaffolds were prepared by suspending the fiber segments at 10% v/v in a 0.1% w/v HHMP solution, packed via centrifugation as previously described, handled for the experiment (e.g., rheology or extrusion), then treated with 2 min of UV light at 5 mW cm⁻² to induce crosslinking of the unquenched methacrylate groups.

4.5 Oscillatory rheological characterization: Mechanical properties of PHMs were determined via rheological measurements (Anton Paar MCR 302 rheometer) using a 25 mm parallel plate geometry, a solvent trap, and a gap height of 200 μm. Shear-thinning and self-healing properties were determined using oscillatory time sweeps that cyclically changed between low strain (1%) and high strain (250%) at 1 Hz. To assess the ink/support properties of PHMs for extrusion-based printing applications, strain sweeps (0.01-250%, 1 Hz) were conducted to elucidate yield strains (%). Rheological measurements were conducted in triplicate. Shear recovery for each dilution was analyzed using a paired T-test and dilutions compared against each other were assessed using a one-way ANOVA coupled with a Tukey HSD post-hoc test to determine statistical significance.

For characterization of PHM viscoelasticity, time sweeps were utilized to quantify storage and loss moduli. To assess the ability of PHMs to stress relax, a constant 15% strain was applied to the system and the resultant shear stress was recorded over time. Stress relaxation plots were smoothed using an exponential smoothing algorithm for clean presentation. For low and high crosslinkable scaffolds, the samples were irradiated for 2 min at 5 mW cm⁻² before conducting the rest of the rheological measurements. Time sweeps for UV crosslinking of PHMs are shown in FIG. 29. Viscoelasticity rheological assessments were conducted in duplicate, with representative data shown.

4.6 Extensional rheological characterization of PHMs: Extensibility of PHMs was determined via a modified filament stretching extensional rheology protocol (FiSER, Anton Paar MCR 302 rheometer) using a 25 mm parallel plate geometry. Briefly, 500 μl of PHM scaffold was added to the rheometer stage and the gap height was lowered to 1 mm. A vertical strain rate of \dot{∈}=1.2 s⁻¹ was applied, and the normal force was tracked as the geometry height was increased. Filament failure was determined at the point where the material completely broke, and the height of failure was utilized to quantify the overall percentage stretch to failure relative to the original height. FiSER experiments were conducted in triplicate, and statistical significance was evaluated using a one-way ANOVA coupled with a Tukey HSD post-hoc test.

4.7 Extrusion printing of PHMs: A FELIX BIOprinter was used for all controlled extrusion of PHMs, and PHM-100 was utilized as the ink due to its robust mechanical properties compared to other dilutions. Inks were loaded into 1 ml syringes (BD) equipped with a 22 G needle via centrifugation at 200 RCF for 1 min to ensure all air bubbles were removed prior to printing. Printer workflows were written using G-code commands that were actuated through the FELIX BIOprinter's software interface. Targeting filaments with 500 μm diameter, macroscale properties were determined by manipulation of the filament, and microscale properties were determined by printing PHM-100 on glass coverslips. For printed filaments used in cell culture, the glass coverslips were first modified to present methacrylate groups based on a previously described protocol to allow for covalent conjugation of the filament to the coverslip.

4.8 Cell culture: Immortalized murine myoblasts (C2C12 s, ATCC) were used for cell culture experiments (passages 6-7). Cells were cultured in standard growth media comprised of high glucose Dulbeccos's Modified Eagle's Medium (DMEM), 10% v/v fetal bovine serum (Gibco), and 1% v/v antibiotic/antimycotic (Gibco). Media was changed every 2 days during expansion and experiments.

For experiments with cells seeded atop printed filaments, the crosslinked scaffolds were first sterilized via irradiation with germicidal light for 2 h. Scaffolds were then hydrated in complete growth media for 1 h prior to seeding cells at a density of 1×10¹ cells per scaffold. Cells were cultured for 2 d prior to fixing and staining for analysis. For experiments with cells seeded within fibers as a support, fiber solutions (10% v/v in PBS) were sterilized under germicidal light for 2 h, packed via centrifugation, and the PBS supernatant was exchanged with sterile C2C12 growth media for at least 2 h prior to packing for cell experiments. PHM-100 was prepared as described above and C2C12 s were gently mixed into PHMs at a density of 1×10⁷ cells/ml and cultured for 1 d in a PDMS mold prior to fixing and staining.

4.9 Cell staining: Prior to cell staining, C2C12 s seeded on printed filaments were fixed in 10% v/v neutral buffered formalin for 15 min, permeabilized in 0.1% v/v Triton X-100 for 10 min, then blocked with 3% w/v bovine serum albumin for 2 h at room temperature. Cells were incubated with AlexaFluor-488 phalloidin for 1 h to visualize F-actin (1:600, Invitrogen). Samples were washed thrice in PBS to remove unbound molecules.

A similar protocol was utilized for experiments with C2C12 s in PHMs supports. Cells were fixed for 1 h, permeabilized for 1 h, and blocked for 2 h using the same solution concentrations prior to tagging F-actin with AlexaFluor-488 phalloidin (1:200) for 2 h. Again, samples were washed in PBS thrice to remove unbound fluorophore.

Microscopy and image analysis: Fluorescence microscopy for fiber segmentation and printed filament analysis was conducted on a Leica DMi8 widefield fluorescent microscope. For cell imaging on filaments or within supports, a Leica Stellaris Confocal microscope was utilized to image Z-stacks, with representative maximum projections shown here. Fiber length, diameter, and directionality, as well as cell orientation and area were all quantified using built-in ImageJ functionalities.

The stress relaxation profiles of PHMs exhibit behaviors similar to viscoelastic solids. Interestingly, work by Wingert and coworkers demonstrated that Nylon-11 nanofiber meshes dissipated stress similarly to PHMs, albeit at much longer time scales (on the order of minutes). Therefore, the same viscoelastic standard linear solid (SLS) model was leveraged to analyze the stress relaxation of PHMs that was utilized to model the Nylon-11 fibers. The basic SLS model is shown below as Equation 1, and the resultant τ values are shown in the figure corresponding to the PHM group.

Where:

$$\frac{σ ( t )}{{σ}_{0}} = β + ( 1 - β ) exp ( - \frac{t}{τ} ) \qquad ( 1 )$$

$$τ = relaxation time constant$$

$$β = {lim}_{t \to ∞} \frac{σ ( t )}{{σ}_{0}}$$

1. A scaffold comprising polymeric fibers having an aspect ratio of at least 20 and hydrogel microparticles, wherein the polymeric fibers and the hydrogel microparticles form a stable network.

2. The scaffold of claim 1, having a porosity of at least 30%.

3. (canceled)

4. The scaffold of claim 1, wherein the polymeric fibers are not crosslinked.

5. (canceled)

6. The scaffold of claim 1, wherein the polymeric fibers comprise a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile, or combinations thereof.

7. (canceled)

8. (canceled)

9. (canceled)

10. The scaffold of claim 1, wherein the polymeric fibers have an aspect ratio of at least 30.

11. (canceled)

12. (canceled)

13. (canceled)

14. The scaffold of claim 1, wherein the hydrogel microparticles comprises a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile, or combinations thereof.

15. (canceled)

16. (canceled)

17. (canceled)

18. A method of producing a stable scaffold, comprising mixing polymeric fibers having an aspect ratio of at least 20 and hydrogel microparticles, thereby the polymeric fibers and the hydrogel microparticles form a stable network.

19. (canceled)

20. (canceled)

21. A method of culturing cells, comprising:
mixing the cells with the scaffold of claim 1, thereby the cells are embedded in the scaffold, and
culturing the cells embedded in the scaffold.

22. (canceled)

23. The method of claim 21, The method further comprises, prior to culturing the cells embedded in the scaffold, extruding the scaffold having embedded cells.

24. (canceled)

25. (canceled)

26. A hydrogel comprising polymeric fibers having a mean length of at least 35 μm, wherein the hydrogel is stable and has a porosity of at least 30%.

27. (canceled)

28. The hydrogel of claim 26, wherein the polymeric fibers comprise a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile, or combinations thereof.

29. The hydrogel of claim 26, wherein the polymeric fibers are not crosslinked.

30. (canceled)

31. (canceled)

32. The hydrogel of claim 26, wherein the polymeric fibers are electrospun fibers.

33. (canceled)

34. (canceled)

35. The hydrogel of claim 26, comprising about 10% to about 80% by volume the polymeric fibers.

36. A scaffold comprising the hydrogel of claim 26.

37. An ink or filament for 3D printing comprising the hydrogel of claim 26.

38. (canceled)

39. (canceled)

40. A method of preparing a hydrogel, the method comprising:
electrospinning a polymer into a polymeric fiber having a mean length of at least 35 μm; and
hydrating the polymeric fiber in an aqueous medium to form a stable hydrogel having a porosity of at least 30%.

41. The method of claim 40, comprising electrospinning a polymer into a polymeric fiber and segmenting the polymeric fiber, the segmented polymeric fiber having a mean length of at least 35 μm.

42. A process of 3D printing, comprising printing the ink or filament of claim 37 using a 3D printer.

43. A method of culturing cells, the method comprising
mixing the cells with the scaffold of claim 36, thereby the cells are embedded in the scaffold; and
culturing the cells embedded in the scaffold.

44. (canceled)
